(19) United States
(12) Patent Application Publication
(10) Pub. No.: US 2010/0297194 A1
(43) Pub. Date: Nov. 25, 2010

(54) FORMULATION FOR ORAL ADMINISTRATION OF APOPTOSIS PROMOTER

(76) Inventors: Nathaniel Catron, Vernon Hills, IL (US); Michael G. Fickes, Evanston, IL (US); Cristina M. Fischer, Wadsworth, IL (US); Rajeev Gokhale, Libertyville, IL (US); Anthony R. Haight, Wadsworth, IL (US); Katherine Heemstra, Chicago, IL (US); David Hill, Gurnee, IL (US); Martin Knobloch, Neuhofen (DE); Drazen Kostelac, Roemerberg (DE); Justin S. Lafountaine, Chicago, IL (US); Yanxia Li, Grayslake, IL (US); Bernd Liepold, Dossenheim (DE); Kennan Marsh, Lake Forest, IL (US); Jonathan M. Miller, Lindenhurst, IL (US); Claudia Packhaeuser, Munich (DE); Yeshwant D. Sanzgiri, Gurnee, IL (US); Eric A. Schmitt, Libertyville, IL (US); Yi Shi, Libertyville, IL (US); Norbert Steiger, Lingenfeld (DE); Ping Tong, Libertyville, IL (US); Huailiang Wu, Long Grove, IL (US); Geoff G.Z. Zhang, Libertyville, IL (US); Deliang Zhou, Vernon Hills, IL (US)

Correspondence Address: Harness Dickey & Pierce, PLC, 7700 Bonhomme, Suite 400, Clayton, MO, 63105, US

(21) Appl. No.: 12/770,299

(22) Filed: Apr. 29, 2010

Related U.S. Application Data
(60) Provisional application No. 61/174,299, filed on Apr. 30, 2009.
(60) Provisional application No. 61/174,318, filed on Apr. 30, 2009.
(60) Provisional application No. 61/185,105, filed on Jun. 8, 2009.
(60) Provisional application No. 61/185,130, filed on Jun. 8, 2009.
(60) Provisional application No. 61/218,281, filed on Jun. 18, 2009.
(60) Provisional application No. 61/289,254, filed on Dec. 22, 2009.
(60) Provisional application No. 61/289,289, filed on Dec. 22, 2009.

(51) Int. Cl. A61K 31/5377; A61K 33/04; A61K 9/14; A61P 35/00

(52) U.S. Cl. 424/400; 514/235.8; 424/711; 424/709

(57) ABSTRACT

An orally deliverable pharmaceutical composition comprises as a sole or first active ingredient a compound of Formula I defined herein or a pharmaceutically acceptable salt thereof, for example ABT-263 free base or ABT-263 bis-HCl salt, dispersed, in a free base equivalent amount of at least about 2.5% by weight of the composition, in a pharmaceutically acceptable carrier; wherein said active ingredient is in solid-state form and/or the composition further comprises, dispersed in the carrier, a pharmaceutically acceptable heavier-chalcogen antioxidant in an amount effective to inhibit oxidation of the active ingredient at a thioether linkage thereof. The composition is suitable for oral administration to a subject in need thereof for treatment of a disease characterized by overexpression of one or more anti-apoptotic Bcl-2 family proteins, for example cancer.

### CROSS-REFERENCE TO RELATED APPLICATIONS

This application claims priority benefit of U.S. Provisional Application Ser. No. 61/174,299 filed on Apr. 30, 2009, Ser. No. 61/174,318 filed on Apr. 30, 2009, Ser. No. 61/185,105 filed on Jun. 8, 2009, Ser. No. 61/185,130 filed on Jun. 8, 2009, Ser. No. 61/218,281 filed on Jun. 18, 2009, Ser. No. 61/289,254 filed on Dec. 22, 2009, and Ser. No. 61/289,289 filed on Dec. 22, 2009.

Cross-reference is made to the following co-filed U.S. applications containing subject matter related to the present application: Ser. No. 12/______ titled “Lipid formulation of apoptosis promoter”, which claims priority benefit of U.S. provisional application Ser. No. 61/174,245 filed on Apr. 30, 2009; Ser. No. 12/______ titled “Salt of ABT-263 and solid-state forms thereof”, which claims priority benefit of U.S. provisional application Ser. No. 61/174,274 filed on Apr. 30, 2009; Ser. No. 12/______ titled “Stabilized lipid formulation of apoptosis promoter”, which claims priority benefit of above-referenced U.S. provisional application Ser. No. 61/174,299 and Ser. No. 61/289,254; and Ser. No. 12/______ titled “Solid oral formulation of ABT-263”, which claims priority benefit of above-referenced U.S. provisional application Ser. No. 61/174,318.

The entire disclosure of each of the above applications is incorporated herein by reference.

### FIELD OF THE INVENTION

The present invention relates to pharmaceutical compositions comprising an apoptosis-promoting agent, for example ABT-263, and to methods of use thereof for treating diseases characterized by overexpression of anti-apoptotic Bcl-2 family proteins. More particularly the invention relates to such compositions that exhibit improved stability and adequate oral bioavailability, and to oral dosage regimens for administration of such a composition to a subject in need thereof.

### BACKGROUND OF THE INVENTION

Evasion of apoptosis is a hallmark of cancer (Hanahan & Weinberg (2000) Cell 100:57-70). Cancer cells must overcome a continual bombardment by cellular stresses such as DNA damage, oncogene activation, aberrant cell cycle progression and harsh microenvironments that would cause normal cells to undergo apoptosis. One of the primary means by which cancer cells evade apoptosis is by up-regulation of anti-apoptotic proteins of the Bcl-2 family.

Compounds that occupy the BH3 binding groove of Bcl-2 proteins have been described, for example by Bruncko et al. (2007) J. Med. Chem. 50:641-662. These compounds have included N-(4-(4-((4′-chloro-(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide, otherwise known as ABT-737, which has the formula:

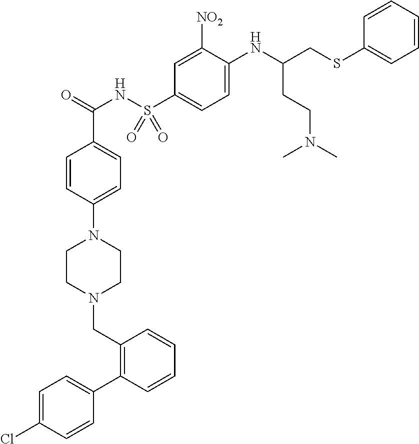

ABT-737 binds with high affinity (<1 nM) to proteins of the Bcl-2 family (specifically Bcl-2, Bcl-X_{L} and Bcl-w). It exhibits single-agent activity against small-cell lung cancer (SCLC) and lymphoid malignancies, and potentiates pro-apoptotic effects of other chemotherapeutic agents. ABT-737 and related compounds, and methods to make such compounds, are disclosed in U.S. Patent Application Publication No. 2007/0072860 of Bruncko et al.

More recently, a further series of compounds has been identified having high binding affinity to Bcl-2 family proteins. These compounds, and methods to make them, are disclosed in U.S. Patent Application Publication No. 2007/0027135 of Bruncko et al. (herein “the '135 publication”), incorporated by reference herein in its entirety, and can be seen from their formula to be structurally related to ABT-737.

One compound, identified as “Example 1” in the '135 publication, is N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trffluoromethyl)sulfonyl)benzene-sulfonamide, otherwise known as ABT-263. This compound has a molecular weight of 974.6 g/mol and has the formula:

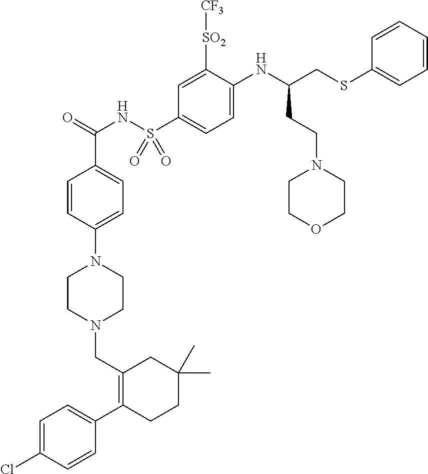

The '135 publication states that while inhibitors of Bcl-2 family proteins previously known may have either potent cellular efficacy or high systemic exposure after oral administration, they do not possess both properties. A typical measure of cellular efficacy of a compound is the concentration eliciting 50% cellular effect (EC₅₀). A typical measure of systemic exposure after oral administration of a compound is the area under the curve (AUC) resulting from graphing plasma concentration of the compound versus time from oral administration. Previously known compounds, it is stated in the '135 publication, have a low AUC/EC₅₀ ratio, meaning that they are not orally efficacious. By contrast, compounds provided therein are stated to demonstrate enhanced properties with respect to cellular efficacy and systemic exposure after oral administration, resulting in a AUC/EC₅₀ ratio significantly higher than that of previously known compounds.

ABT-263 binds with high affinity (<1 nM) to Bcl-2 and Bcl-X_{L} and is believed to have similarly high affinity for Bcl-w. Its AUC/EC₅₀ ratio is reported in the '135 publication as 56, more than an order of magnitude greater than that reported for ABT-737 (4.5). For determination of AUC according to the '135 publication, each compound was administered to rats in a single 5 mg/kg dose by oral gavage as a 2 mg/ml solution in a vehicle of 10% DMSO (dimethyl sulfoxide) in PEG-400 (polyethylene glycol of average molecular weight about 400).

Oral bioavailability (as expressed, for example, by AUC after oral administration as a percentage of AUC after intravenous administration) is not reported in the '135 publication, but can be concluded therefrom to be, at least in a rat model, substantially greater for ABT-263 than for ABT-737, when administered in PEG-400/DMSO solution.

Various solutions to the challenge of low oral bioavailability have been proposed in the art. For example, U.S. Pat. No. 5,645,856 to Lacy et al. proposes formulating a hydrophobic drug with (a) an oil, (b) a hydrophilic surfactant and (c) a lipophilic surfactant that substantially reduces an inhibitory effect of the hydrophilic surfactant on in vivo lipolysis of the oil, such lipolysis being said to be a factor promoting bioavailability of the drug. Among numerous classes of hydrophilic surfactants listed are phospholipids such as lecithins.

U.S. Pat. No. 6,267,985 to Chen & Patel is directed, inter alia, to a pharmaceutical composition comprising (a) a triglyceride, (b) a carrier comprising at least two surfactants, one of which is hydrophilic, and (c) a therapeutic agent capable of being solubilized in the triglyceride, the carrier or both. It is specified therein that the triglyceride and the surfactants must be present in amounts providing a clear aqueous dispersion when the composition is mixed with an aqueous solution under defined conditions. Among extensive separate lists of exemplary ingredients, mention is made of “glyceryl tricaprylate/caprate” as a triglyceride, and phospholipids including phosphatidylcholine as surfactants.

U.S. Pat. No. 6,451,339 to Patel & Chen mentions disadvantages of presence of triglycerides in such compositions, and proposes otherwise similar compositions that are substantially free of triglycerides, but that likewise provide clear aqueous dispersions.

U.S. Pat. No. 6,309,663 to Patel & Chen proposes pharmaceutical compositions comprising a combination of surfactants said to enhance bioabsorption of a hydrophilic therapeutic agent. Phospholipids such as phosphatidylcholine are again listed among exemplary surfactants.

U.S. Pat. No. 6,464,987 to Fanara et al. proposes a fluid pharmaceutical composition comprising an active substance, 3% to 55% by weight of phospholipid, 16% to 72% by weight of solvent, and 4% to 52% by weight of fatty acid. Compositions comprising Phosal 50 PG™ (primarily comprising phosphatidylcholine and propylene glycol), in some cases together with Phosal 53 MCT™ (primarily comprising phosphatidylcholine and medium chain triglycerides), are specifically exemplified. Such compositions are said to have the property of gelling instantaneously in presence of an aqueous phase and to allow controlled release of the active substance.

U.S. Pat. No. 5,538,737 to Leonard et al. proposes a capsule containing a water-in-oil emulsion wherein a water-soluble drug salt is dissolved in the water phase of the emulsion and wherein the oil phase comprises an oil and an emulsifying agent. Among oils mentioned are medium chain triglycerides; among emulsifying agents mentioned are phospholipids such as phosphatidylcholine. Phosal 53 MCT™, which contains phosphatidylcholine and medium chain triglycerides, is reportedly used according to various examples therein.

U.S. Pat. No. 5,536,729 to Waranis & Leonard proposes an oral formulation comprising rapamycin, at a concentration of about 0.1 to about 50 mg/ml, in a carrier comprising a phospholipid solution. It is stated therein that a preferred formulation can be made using Phosal 50 PG™ as the phospholipid solution. An alternative phospholipid solution mentioned is Phosal 50 MCT™.

U.S. Pat. No. 5,559,121 to Harrison et al. proposes an oral formulation comprising rapamycin, at a concentration of about 0.1 to about 100 mg/ml, in a carrier comprising N,N-dimethylacetamide and a phospholipid solution. Examples of the more preferred embodiments are shown to be prepared using Phosal 50 PG™. An alternative phospholipid solution mentioned is Phosal 50 MCT™.

U.S. Patent Application Publication No. 2007/0104780 of Lipari et al. discloses that a small-molecule drug (defined therein as having molecular weight, excluding counterions in the case of salts, not greater than about 750 g/mol, typically not greater than about 500 g/mol) having low water solubility can be formulated as a solution in a substantially non-aqueous carrier comprising at least one phospholipid and a pharmaceutically acceptable solubilizing agent. The solution, when mixed with an aqueous phase, is said to form a non-gelling, substantially non-transparent liquid dispersion. Illustratively, formulations of N-(4-(3-amino-1H-indazol-4-yl)phenyl)-N′-(2-fluoro-5-methylphenyl)urea (the protein tyrosine kinase inhibitor ABT-869) comprising Phosal 53 MCT™ and other ingredients are described therein.

Recently, Tse et al. (2008) Cancer Res. 68(9):3421-3428, reported in supplementary data thereto that, in a dog model, oral bioavailability of an ABT-263 solution in PEG-400/DMSO was 22.4%, and that of an ABT-263 solution in 60% Phosal™ PG (phosphatidylcholine+propylene glycol), 30% PEG-400 and 10% ethanol was 47.6%.

At the time of the present invention, however, the art was silent as to whether compounds of the '135 publication such as ABT-263 have sufficient chemical stability to permit formulation in pharmaceutical compositions suitable as storable, transportable materials of commerce as opposed to extemporaneously prepared solutions. Further, the art gave no indication as to whether, if such compositions could be made, they would have acceptable oral bioavailability. Still further, the art was silent as to whether, if such compositions could be made having acceptable oral bioavailability, they could have a concentration of active ingredient sufficient to provide therapeutically effective daily dosing without the need to swallow an unacceptably large volume of liquid or an unacceptably large number of discrete solid dosage forms such as capsules or tablets.

Oxidation reactions represent an important degradation pathway of pharmaceuticals, especially when formulated in solution. A large body of information is available on oxidative mechanisms, but relatively few studies have been performed with specific drugs. Hovorka & Schoneich (2001) J. Pharm. Sci. 90:253-269 have stated that this lack of pharmaceutically relevant data leads to poor predictive ability with respect to drug oxidation between manufacture and administration of formulations of oxidizable drugs, and a consequently uninformed, largely empirical utilization of antioxidants in formulations.

Oxidation can occur by a number of pathways, including uncatalyzed autoxidation of a substrate by molecular oxygen, photolytic initiation, hemolytic thermal cleavage, and metal catalysis. Various functional groups show particular sensitivity towards oxidation. In particular, thioethers can degrade via hydrogen abstraction at the α-position to the sulfur atom or by addition of an α-peroxyl radical directly or via a one-electron transfer process, which transforms a sulfide to a sulfine, sulfone, or sulfoxide (Hovorka & Schoneich, supra).

A particular type of disease for which improved therapies are needed is non-Hodgkin's lymphoma (NHL). NHL is the sixth most prevalent type of new cancer in the U.S. and occurs primarily in patients 60-70 years of age. NHL is not a single disease but a family of related diseases, which are classified on the basis of several characteristics including clinical attributes and histology.

One method of classification places different histological subtypes into two major categories based on natural history of the disease, i.e., whether the disease is indolent or aggressive. In general, indolent subtypes grow slowly and are generally incurable, whereas aggressive subtypes grow rapidly and are potentially curable. Follicular lymphomas are the most common indolent subtype, and diffuse large-cell lymphomas constitute the most common aggressive subtype. The oncoprotein Bcl-2 was originally described in non-Hodgkin's B-cell lymphoma.

Treatment of follicular lymphoma typically consists of biologically-based or combination chemotherapy. Combination therapy with rituximab, cyclophosphamide, doxorubicin, vincristine and prednisone (R-CHOP) is routinely used, as is combination therapy with rituximab, cyclophosphamide, vincristine and prednisone (RCVP). Single-agent therapy with rituximab (targeting CD20, a phosphoprotein uniformly expressed on the surface of B-cells) or fludarabine is also used. Addition of rituximab to chemotherapy regimens can provide improved response rate and increased progression-free survival.

Radioimmunotherapy agents, high-dose chemotherapy and stem cell transplants can be used to treat refractory or relapsed non-Hodgkin's lymphoma. Currently, there is not an approved treatment regimen that produces a cure, and current guidelines recommend that patients be treated in the context of a clinical trial, even in a first-line setting.

First-line treatment of patients with aggressive large B-cell lymphoma typically consists of rituximab, cyclophosphamide, doxorubicin, vincristine and prednisone (R-CHOP), or dose-adjusted etoposide, prednisone, vincristine, cyclophosphamide, doxorubicin and rituximab (DA-EPOCH-R).

Most lymphomas respond initially to any one of these therapies, but tumors typically recur and eventually become refractory. As the number of regimens patients receive increases, the more chemotherapy-resistant the disease becomes. Average response to first-line therapy is approximately 75%, 60% to second-line, 50% to third-line, and about 35-40% to fourth-line therapy. Response rates approaching 20% with a single agent in a multiple relapsed setting are considered positive and warrant further study.

Current chemotherapeutic agents elicit their antitumor response by inducing apoptosis through a variety of mechanisms. However, many tumors ultimately become resistant to these agents. Bcl-2 and Bcl-X_{L} have been shown to confer chemotherapy resistance in short-term survival assays in vitro and, more recently, in vivo. This suggests that if improved therapies aimed at suppressing the function of Bcl-2 and Bcl-X_{L} can be developed, such chemotherapy-resistance could be successfully overcome.

Apoptosis-promoting drugs that target Bcl-2 family proteins such as Bcl-2 and Bcl-X_{L} are best administered according to a regimen that provides continual, for example daily, replenishment of the plasma concentration, to maintain the concentration in a therapeutically effective range. This can be achieved by daily parenteral, e.g., intravenous (i.v.) or intraperitoneal (i.p.) administration. However, daily parenteral administration is often not practical in a clinical setting, particularly for outpatients. To enhance utility of an apoptosis-promoting agent, whether in a clinical or community setting, for example as a chemotherapeutic in cancer patients, an orally bioavailable dosage form having sufficient storage-stability not to be limited to extemporaneous preparation would be highly desirable. Such a dosage form, and a regimen for oral administration thereof, would represent an important advance in treatment of many types of cancer, including non-Hodgkin's lymphoma, and would more readily enable combination therapies with other chemotherapeutics.

### SUMMARY OF THE INVENTION

As reported in the '135 publication, oral bioavailability of a dilute (2 mg/ml) solution of ABT-263 free base in PEG-400/DMSO in a rat model is around 20%. Tse et al. (2008), supra, report that a similar solution has comparable bioavailability of around 20% in other species, including dog and monkey, but that improved bioavailability is obtainable, at least in a dog model, by use of a lipid carrier, namely phosphatidylcholine/propylene glycol/PEG-400/ethanol. The concentrations of ABT-263 free base in the PEG-400/DMSO and lipid carriers as tested in dogs are not reported by Tse et al., but are disclosed herein to have been 5 and 10 mg/ml (approximately 0.5% and 1% by weight) respectively.

Recent U.S. Patent Application Publication No. 2009/0149461 of Krivoshik (“the '461 publication”), incorporated herein by reference in its entirety without admission that it constitutes prior art to the present application, reports a Phase 1 clinical trial of ABT-263, formulated extemporaneously as a 25 mg/ml solution in Phosal 53 MCT™ (a proprietary product described hereinafter) and ethanol. It is predicted therein, based on preclinical evidence, that therapeutically effective doses of ABT-263 in human patients will be 200-350 mg/day (see the '461 publication at paragraph [0017] bridging pp. 1-2 and paragraph [0032] on p. 3).

Given the variation in individual patients' body weight, therapeutic response and tolerance of side-effects, as well as variation in bioavailability of different formulations, a suitable daily dose for most patients is likely to be found in a range of about 50 to about 500 mg, more typically about 200 to about 400 mg. Illustratively, to deliver per os 200-400 mg of ABT-263 in the form of a 10 mg/ml (approximately 1% by weight) solution in a lipid carrier requires administration of 20-40 ml of solution per day. If encapsulated in easy-to-swallow liquid-filled capsules, each containing 0.5 ml, this amounts to 40 capsules per day at a 200 mg dose and 80 capsules per day at a 400 mg dose. This is highly inconvenient for the patient and caregiver, and is likely to result in poor patient compliance. A 25 mg/ml (approximately 2.5% by weight) ABT-263 concentration, as used in the study reported in the '461 publication, represents a minimum threshold for clinical acceptability, requiring daily administration of 8-16 ml of solution, or 16-32 capsules each containing 0.5 ml. Further increasing the concentration of active ingredient to provide a less voluminous dosage form, without excessively sacrificing oral bioavailability, is therefore an important desideratum. However, the physical properties of ABT-263, including its low solubility in aqueous and many non-aqueous solvents, make this a significant technical challenge.

Compounding the difficulty of formulating compounds of the '135 publication such as ABT-263, other than as an extemporaneously prepared solution, is the finding that such compounds are susceptible to oxidation, for example in presence of oxygen or reactive oxygen species such as superoxide, hydrogen peroxide or hydroxyl radicals. The term “extemporaneously prepared” herein means preparation not more than one month before, for example not more than one week before, not more than one day before, or immediately before, administration to a patient in need thereof. If a formulation is to have acceptable storage-stability for longer than about one month, a solution to the challenge of oxidative degradation of the active ingredient is required.

The (phenylsulfanyl)methyl group of compounds of the '135 publication have a thioether linkage, which is now known to be susceptible to oxidation, for example in presence of oxygen or reactive oxygen species such as superoxide, hydrogen peroxide or hydroxyl radicals. The above-referenced '135 publication includes antioxidants in an extensive list of excipients said to be useful for administering such compounds.

A number of novel and unexpected findings have led, at least in part, to the present invention. These include the following:
Lipid solution compositions of compounds of the '135 publication such as ABT-263 or a salt thereof are, as indicated above, susceptible to oxidative degradation of the active ingredient. Not all antioxidants are effective to inhibit this oxidative degradation. However, it has been found that a particular class of antioxidants, described herein as “heavier-chalcogen antioxidants” or “HCAs”, are useful if included in an antioxidant-effective amount.
The requirement to maintain in a physically stable liquid formulation not only the active ingredient but, additionally, an HCA in an antioxidant-effective amount can further limit the choice of liquid carrier, particularly for higher active ingredient loadings, for example about 50 mg/ml or higher.
Compounds of the '135 publication such as ABT-263 or a salt thereof in solid-state form are typically less susceptible to oxidative degradation than in solution form. Providing the carrier also in solid-state form, for example as a polymeric matrix wherein solid-state active ingredient is dispersed, or as a dry-blend or granulated mixture of excipients including at least a diluent and a disintegrant, is therefore another approach to inhibiting oxidative degradation.
Solid dispersion formulations comprising a compound of the '135 publication such as ABT-263 or a salt thereof in an amorphous form, dispersed in a polymeric matrix, can be prepared at active ingredient loadings of up to about 25% by weight or even higher. Such formulations exhibit acceptable resistance to oxidative degradation and, if they contain a suitable surfactant to solubilize the active ingredient in gastrointestinal fluid upon release from the matrix, are found to have acceptable oral bioavailability in a dog model.
Remarkably for such a poorly water-soluble drug, ABT-263 or a salt thereof formulated as a conventional dry-blend or granulated mixture with excipients including at least a diluent and a disintegrant at an active ingredient loading of up to about 40% by weight or even higher, exhibits generally acceptable oral bioavailability. Even more remarkably, particle size reduction is not essential to achieving acceptable bio availability, although it can provide more rapid release of the active ingredient.
As an alternative liquid formulation, a suspension of crystalline active ingredient (for this purpose a crystalline salt such as ABT-263 bis-HCl is preferred) can be prepared in an aqueous carrier, at ABT-263 free base equivalent concentrations of at least about 25 mg/ml, for example about 50 mg/ml or higher, by appropriate selection of surfactant as a suspending agent. Particle size reduction to provide a D₉₀ not greater than about 2 μm, for example not greater than about 1 μm, provides a nanosuspension having remarkably high oral bioavailability, comparable to that of a lipid solution formulation.

In accordance with these findings, there is now provided an orally deliverable pharmaceutical composition comprising as a sole or first active ingredient a compound of Formula I

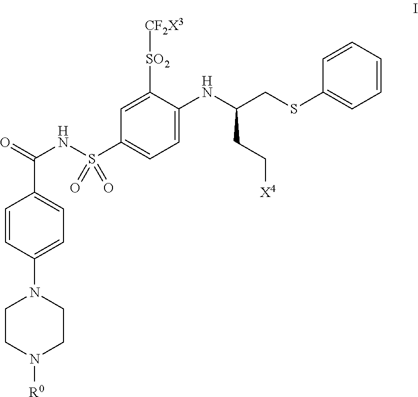

where X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is

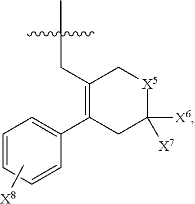

where X⁵ is —CH₂—, —C(CH₃)₂— or —CH₂CH₂—; X⁶ and X⁷ are both —H or both methyl; and X⁸ is fluoro, chloro, bromo or iodo; or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is

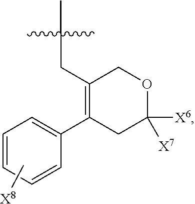

where X⁶, X⁷ and X⁸ are as above; or
(3) X⁴ is morpholin-4-yl or —N(CH₃)₂; and R⁰ is

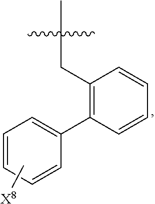

where X⁸ is as above;
   or a pharmaceutically acceptable salt thereof, dispersed, in a free base equivalent amount of at least about 2.5% by weight of the composition, in a pharmaceutically acceptable carrier; wherein said active ingredient is in solid-state form and/or the composition further comprises, dispersed in the carrier, a pharmaceutically acceptable HCA in an amount effective to inhibit oxidation of the active ingredient at a thioether linkage thereof.

In some embodiments, the sole or first active ingredient is ABT-263 or a pharmaceutically acceptable salt thereof, for example ABT-263 free base or ABT-263 bis-hydrochloride salt (ABT-263 bis-HCl).

According to such embodiments, it is preferred that the carrier should comprise excipients selected to provide sufficient bioavailability of ABT-263 to be therapeutically effective for promotion of apoptosis when orally administered to a non-fasting human subject in need thereof in a daily dosage amount of about 200 to about 400 mg ABT-263 free base equivalent. “Sufficient bioavailability” in this context can be evidenced, for example, by
bioavailability of at least about 15% in a non-fasting dog model;
one or both of (a) an ABT-263 AUC₀₋₂₄ of at least about 20 μg·h/ml, and/or (b) an ABT-263 Cₘₐₓ of at least about 2.5 μg/ml, in a single-dose non-fasting human pharmacokinetic study at an ABT-263 free base equivalent dose of about 200 to about 400 mg;
a steady-state ABT-263 Cₘᵢₙ of about 1 to about 5 μg/ml and a steady-state ABT-263 Cₘₐₓ of about 3 to about 8 μg/ml in a non-fasting human pharmacokinetic study at a daily ABT-263 free base equivalent dose of about 200 to about 400 mg; or
at least substantial bioequivalence in a human pharmacokinetic study to a prototype extemporaneously prepared formulation that consists of a 25 mg/ml solution of ABT-263 bis-HCl in a mixture of 90% phosphatidylcholine+medium chain triglycerides 53/29 and 10% ethanol.

In some embodiments, the carrier is liquid, having the active ingredient and a pharmaceutically acceptable HCA in an antioxidant-effective amount in solution or suspension therein.

In other embodiments, the carrier is solid, having the active ingredient dispersed therein in solid-state form. In such embodiments, presence of a pharmaceutically acceptable HCA is optional. The term “solid-state”, as used herein to describe a physical form of the active ingredient, includes crystalline, semi-crystalline, amorphous, and solid or glassy solution forms. Crystalline, semi-crystalline and amorphous forms can be essentially solvent-(including water-) free or can take the form of solvates or hydrates of the active ingredient.

There is further provided a method for treating a disease characterized by apoptotic dysfunction and/or overexpression of an anti-apoptotic Bcl-2 family protein, comprising orally administering to a subject having the disease a therapeutically effective amount of a composition as described above. Examples of such a disease include many neoplastic diseases including cancers. A specific illustrative type of cancer that can be treated according to the present method is non-Hodgkin's lymphoma. Another specific illustrative type of cancer that can be treated according to the present method is chronic lymphocytic leukemia. Yet another specific illustrative type of cancer that can be treated according to the present method is acute lymphocytic leukemia, for example in a pediatric patient.

Additional embodiments of the invention, including more particular aspects of those provided above, will be found in, or will be evident from, the detailed description that follows.

### BRIEF DESCRIPTION OF THE DRAWINGS

FIG. 1 is a schematic phase diagram of ABT-263 free base solutions in ternary “IPT” lipid systems as described in Example 8. The shaded portion of the diagram represents an area of optimized formulation composition.

FIG. 2 is a schematic phase diagram of ABT-263 free base solutions in ternary “IST” lipid systems as described in Example 8. The shaded portion of the diagram represents an area of optimized formulation composition.

FIG. 3 is a graphical representation of ABT-263 plasma concentration over a 24-hour period following oral administration to dogs (non-fasted except where otherwise indicated) of a composition of the invention (Formulation 8) and a comparative solution of ABT-263 bis-HCl in a lipid medium (Formulation C), as described in Example 15.

FIG. 4 is a graphical representation of effects of various surfactants on dissolution rates of solid dispersions containing ABT-263 bis-HCl as described in Example 18.

FIG. 5 is a graphical representation of effects of various surfactants on dissolution rates of solid dispersions containing ABT-263 free base as described in Example 18.

FIG. 6 is a graphical representation of effects of various polymeric carriers on dissolution rates of solid dispersions containing ABT-263 bis-HCl as described in Example 19.

FIG. 7 shows plasma concentration of ABT-263 at different time points following oral administration to fasted or fed dogs of an ABT-263 bis-HCl solid dispersion formulation containing Span™ 20 as solubilizer, at doses of 50, 100 or 200 mg, as described in Example 23.

FIG. 8 shows plasma concentration of ABT-263 at different time points following oral administration to fasted or fed dogs of an ABT-263 bis-HCl solid dispersion formulation containing TPGS as solubilizer, at doses of 50, 100 or 200 mg, as described in Example 23.

FIG. 9 shows plasma concentration of ABT-263 at different time points following oral administration to fed dogs of ABT-263 free base or ABT-263 bis-HCl solid dispersion formulations containing TPGS only, or TPGS+propylene glycol as plasticizer, at a dose of 50 mg, as described in Example 24.

FIGS. 10 and 11 show results of an accelerated stability study using open dishes, wherein the sulfoxide content of different ABT-263 solid dispersion formulations was determined at different time points, as described in Example 25.

FIGS. 12 and 13 show results of an accelerated stability study using closed bottles, wherein the sulfoxide content of different ABT-263 solid dispersion formulations was determined at different time points, as described in Example 25.

FIG. 14 shows release of ABT-263 from tablets containing different ABT-263 solid dispersion formulations, as described in Example 28.

### DETAILED DESCRIPTION

The invention is described herein with specific reference to the following embodiments.

In a first composition embodiment, there is provided an orally deliverable pharmaceutical composition comprising (a) a compound of Formula I as defined hereinabove, or a pharmaceutically acceptable salt thereof, in a free base equivalent amount of at least about 2.5% by weight of the composition; (b) a pharmaceutically acceptable heavier-chalcogen antioxidant (HCA); and (c) a substantially non-aqueous pharmaceutically acceptable carrier that comprises one or more lipids; wherein said compound and the antioxidant are in solution in the carrier.

In a second composition embodiment, there is provided an orally deliverable pharmaceutical capsule comprising a capsule shell having encapsulated therewithin, in an amount not greater than about 1000 mg per capsule, a liquid solution of a compound of Formula I as defined hereinabove, or a pharmaceutically acceptable salt thereof, in a free base equivalent amount of at least about 2.5% by weight of the solution, in a substantially non-ethanolic carrier that comprises as pharmaceutically acceptable excipients:
(a) at least one phospholipid,
(b) at least one solubilizing agent for the at least one phospholipid, selected from the group consisting of glycols, glycolides, glycerides and mixtures thereof,
(c) at least one non-phospholipid surfactant, and
(d) a pharmaceutically acceptable HCA.

In a third composition embodiment, there is provided an orally deliverable liquid pharmaceutical composition comprising an aqueous medium having suspended therein a solid particulate compound having a D₉₀ particle size not greater than about 3 μm; wherein the compound is of Formula I as defined hereinabove, or a pharmaceutically acceptable salt thereof, and is present in a free base equivalent amount of at least about 2.5% by weight of the composition; and wherein the aqueous medium further comprises at least one pharmaceutically acceptable surfactant and at least one pharmaceutically acceptable basifying agent in amounts that are effective together to inhibit particle size increase.

In a fourth composition embodiment, there is provided an orally deliverable solid dispersion comprising, in essentially non-crystalline, for example amorphous, form, a compound of Formula I as defined hereinabove, or a pharmaceutically acceptable salt thereof, in a free base equivalent amount of at least about 2.5% by weight of the composition, dispersed in a solid matrix that comprises (a) a pharmaceutically acceptable water-soluble polymeric carrier and (b) a pharmaceutically acceptable surfactant.

In a fifth composition embodiment, there is provided an orally deliverable pharmaceutical dosage form comprising a solid dispersion or solid solution that comprises (a) a compound of Formula I as defined hereinabove, or a pharmaceutically acceptable salt thereof, in a free base equivalent amount of at least about 2.5% by weight of the composition, (b) at least one pharmaceutically acceptable polymer and (c) at least one pharmaceutically acceptable solubilizer.

In a sixth composition embodiment, there is provided an orally deliverable pharmaceutical composition comprising (a) a compound of Formula I as defined hereinabove, or a pharmaceutically acceptable salt thereof, in solid particulate form and in a free base equivalent amount of at least about 2.5% by weight of the composition, and (b) a plurality of pharmaceutically acceptable excipients including at least a solid diluent and a solid disintegrant.

Variants of these six composition embodiments will be readily envisioned by one of skill in the art reading the present disclosure, such variants being embraced by the present invention. As indicated above, a composition of the present invention is, broadly, an orally deliverable pharmaceutical composition comprising as a sole or first active ingredient a compound of Formula I or a pharmaceutically acceptable salt thereof, dispersed, in a free base equivalent amount of at least about 2.5% by weight of the composition, in a pharmaceutically acceptable carrier; wherein said active ingredient is in solid-state form and/or the composition further comprises, dispersed in the carrier, a pharmaceutically acceptable HCA in an amount effective to inhibit oxidation of the active ingredient at a thioether linkage thereof.

Compositions of any of the above embodiments can be used in a method of the invention for treating a disease characterized by apoptotic dysfunction and/or overexpression of an anti-apoptotic Bcl-2 family protein, for example a neoplastic disease such as cancer. Such a method comprises orally administering to a subject having the disease a therapeutically effective amount of a composition as described herein.

A composition of the invention is “orally deliverable”, i.e., adapted for oral administration; however, such a composition can be useful for delivery of the drug to a subject in need thereof by other routes of administration, including without limitation parenteral, sublingual, buccal, intranasal, pulmonary, topical, transdermal, intradermal, ocular, otic, rectal, vaginal, intragastric, intracranial, intrasynovial and intra-articular routes.

The terms “oral administration” and “orally administered” herein refer to administration to a subject per os (p.o.), that is, administration wherein the composition is immediately swallowed, for example with the aid of a suitable volume of water or other potable liquid. “Oral administration” is distinguished herein from intraoral administration, e.g., sublingual or buccal administration or topical administration to intraoral tissues such as periodontal tissues, that does not involve immediate swallowing of the composition.

A compound of Formula I or salt thereof can be the sole active ingredient in the composition, in which case the compound or salt can be administered in monotherapy or in combination therapy with one or more other drugs formulated separately from the compound of Formula I or salt thereof. Alternatively, a compound of Formula I or salt thereof can be accompanied in the composition by one or more additional drugs, for use in combination therapy. In that case, the compound of Formula I or salt thereof is considered the “first active ingredient” for the purpose of the present disclosure.

Therapeutically active compounds, including salts, useful herein typically have low solubility in water, for example less than about 100 μg/ml, in most cases less than about 30 μg/ml. The present invention can be especially advantageous for drugs that are essentially insoluble in water, i.e., having a solubility of less than about 10 μg/ml. Examples of such drugs are include Biopharmaceutics Classification System (BCS) Class IV drug substances that are characterized by low solubility and low permeability (see “Waiver of in vivo bioavailability and bioequivalence studies for immediate-release solid oral dosage forms based on a biopharmaceutics classification system”, U.S. Department of Health and Human Services, Food and Drug Administration, Center for Drug Evaluation and Research (CDER), August 2000). It will be recognized that aqueous solubility of many compounds is pH-dependent; in the case of such compounds the solubility of interest herein is at a physiologically relevant pH, for example a pH of about 1 to about 8. Thus, in various embodiments, the drug has a solubility in water, at least at one point in a pH range from about 1 to about 8, of less than about 100 μg/ml, for example less than about 30 μg/ml, or less than about 10 μg/ml. Illustratively, ABT-263 has a solubility in water at pH 2 of less than 4 μg/ml.

In one embodiment, the composition comprises a compound of Formula I as defined above, or a pharmaceutically acceptable salt of such a compound.

In a further embodiment, the compound has Formula I where X³ is fluoro.

In a still further embodiment, the compound has Formula I where X⁴ is morpholin-4-yl.

In a still further embodiment, the compound has Formula I where R⁰ is

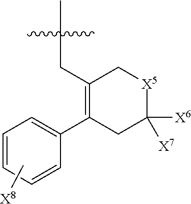

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where R⁰ is

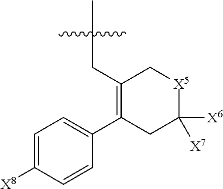

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where X³ is fluoro and X⁴ is morpholin-4-yl.

In a still further embodiment, the compound has Formula I where X³ is fluoro and R⁰ is

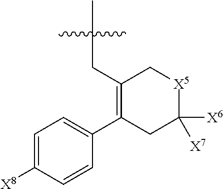

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where X⁴ is morpholin-4-yl and R⁰ is

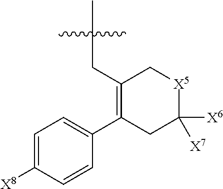

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where X³ is fluoro, X⁴ is morpholin-4-yl and R⁰ is

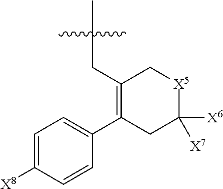

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

Compounds of Formula I may contain asymmetrically substituted carbon atoms in the R- or S-configuration; such compounds can be present as racemates or in an excess of one configuration over the other, for example in an enantiomeric ratio of at least about 85:15. The compound can be substantially enantiomerically pure, for example having an enantiomeric ratio of at least about 95:5, or in some cases at least about 98:2 or at least about 99:1.

Compounds of Formula I may alternatively or additionally contain carbon-carbon double bonds or carbon-nitrogen double bonds in the Z- or E-configuration, the term “Z” denoting a configuration wherein the larger substituents are on the same side of such a double bond and the term “E” denoting a configuration wherein the larger substituents are on opposite sides of the double bond. The compound can alternatively be present as a mixture of Z- and E-isomers.

Compounds of Formula I may alternatively or additionally exist as tautomers or equilibrium mixtures thereof wherein a proton shifts from one atom to another. Examples of tautomers illustratively include keto-enol, phenol-keto, oxime-nitroso, nitro-aci, imine-enamine and the like.

Compounds of Formula I, and methods of preparation of such compounds, are disclosed in the above-cited '135 publication and/or in above-cited U.S. Patent Application Publication No. 2007/0072860, each of which is incorporated herein by reference in its entirety. Terms for substituents used herein are defined exactly as in those publications.

In some embodiments, a compound of Formula I is present in the composition in its parent-compound (“free base”) form, alone or together with a salt form of the compound.

Compounds of Formula I may form acid addition salts, basic addition salts or zwitterions. Salts of compounds of Formula I can be prepared during isolation or following purification of the compounds. Acid addition salts are those derived from reaction of a compound of Formula I with an acid. For example, salts including the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts of a compound of Formula I can be used in a composition of the invention. Basic addition salts including those derived from reaction of a compound with the bicarbonate, carbonate, hydroxide or phosphate of cations such as lithium, sodium, potassium, calcium and magnesium can likewise be used.

A compound of Formula I typically has more than one protonatable nitrogen atom and is consequently capable of forming acid addition salts with more than one, for example about 1.2 to about 2, about 1.5 to about 2 or about 1.8 to about 2, equivalents of acid per equivalent of the compound.

ABT-263 (having Formula I where X³ is fluoro, X⁴ is morpholin-4-yl and R⁰ is

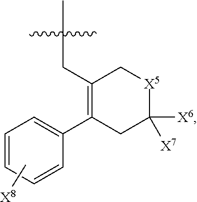

where X⁵ is —C(CH₃)₂—, X⁶ and X⁷ are both —H and X⁸ is chloro) can likewise form acid addition salts, basic addition salts or zwitterions. Salts of ABT-263 can be prepared during isolation or following purification of the compound. Acid addition salts derived from reaction of ABT-263 with an acid include those listed above. Basic addition salts including those listed above can likewise be used. ABT-263 has at least two protonatable nitrogen atoms and is consequently capable of forming acid addition salts with more than one, for example about 1.2 to about 2, about 1.5 to about 2 or about 1.8 to about 2, equivalents of acid per equivalent of the compound.

Illustratively in the case of ABT-263, bis-salts can be formed including, for example, bis-hydrochloride (bis-HCl) and bis-hydrobromide (bis-HBr) salts. These salts can alternatively be called ABT-263 diHCl and ABT-263 diHBr.

For example, ABT-263 bis-HCl, which has a molecular weight of 1047.5 g/mol and is represented by the formula

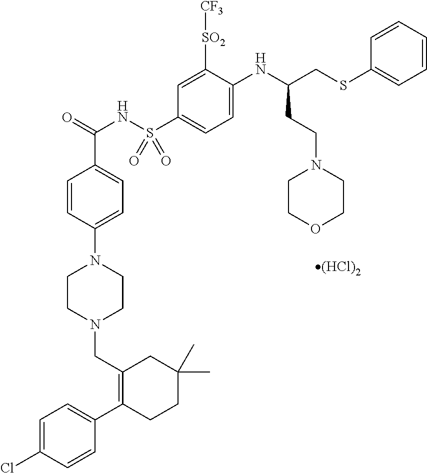

can be prepared by a variety of processes, for example a process that can be outlined as follows.

ABT-263 free base is prepared, illustratively as described in Example 1 of the above-cited '135 publication, the entire disclosure of which is incorporated by reference herein. A suitable weight of ABT-263 free base is dissolved in ethyl acetate. A solution of hydrochloric acid in ethanol (for example about 4.3 kg HCl in 80 g EtOH) is added to the ABT-263 solution in an amount providing at least 2 mol HCl per mol ABT-263 and sufficient EtOH (at least about 20 vol) for crystallization of the resulting ABT-263 bis-HCl salt. The solution is heated to about 45° C. with stiffing and seeds are added as a slurry in EtOH. After about 6 hours, the resulting slurry is cooled to about 20° C. over about 1 hour and is mixed at that temperature for about 36 hours. The slurry is filtered to recover a crystalline solid, which is an ethanol solvate of ABT-263 bis-HCl. Drying of this solid under vacuum and nitrogen with mild agitation for about 8 days yields white desolvated ABT-263 bis-HCl crystals. This material is suitable as active pharmaceutical ingredient (API) for preparation of an ABT-263 bis-HCl formulation of the present invention.

The term “free base” is used for convenience herein to refer to the parent compound, while recognizing that the parent compound is, strictly speaking, zwitterionic and thus does not always behave as a true base. ABT-263 bis-HCl can be prepared by any process that comprises reacting ABT-263 free base with 2 moles of hydrochloric acid (HCl) in a suitable medium.

As indicated above, ABT-263 free base can be prepared by a process as described in Example 1 of the above-cited '135 publication. The product of this process is an amorphous, glassy solid. A powder can be prepared from this product, for example by freeze-drying or precipitation techniques. Such a powder can be used as API in preparing a composition of the present invention; however, it will generally be found preferable to use a crystalline form of ABT-263 free base as API. Such crystalline forms include solvates and solvent-free crystalline forms.

Solvates of ABT-263 free base can be prepared as described below. The starting product can be any solid-state form of ABT-263 free base, including the amorphous form prepared according to the '135 publication.

A measured amount of ABT-263 free base (as indicated, any solid-state form can be used) is suspended in any of a number of solvents or solvent mixtures, including without limitation 2-propanol, 1-propanol, ethyl acetate/ethanol 1:3 v/v, methyl acetate/hexanes 1:1 v/v, chloroform, methanol, 1,4-dioxane/hexanes 1:2 v/v, toluene and benzene. The resulting suspension is agitated at ambient temperature, while protected from light. After a period of time sufficient to permit solvation of ABT-263 free base in each case, crystals are harvested by filter centrifugation. The resulting solvates can be characterized by powder X-ray diffraction (PXRD), for example using a G3000 diffractometer (Inel Corp., Artenay, France) equipped with a curved position-sensitive detector and parallel-beam optics. The diffractometer is operated with a copper anode tube (1.5 kW fine focus) at 40 kV and 30 mA. An incident-beam germanium monochromator provides monochromatic radiation. The diffractometer is calibrated using an attenuated direct beam at one-degree intervals. Calibration is checked using a silicon powder line position reference standard (NIST 640c). The instrument is computer-controlled using Symphonix software (Inel Corp., Artenay, France) and the data are analyzed using Jade software (version 6.5, Materials Data, Inc., Livermore, Calif.). The sample is loaded onto an aluminum sample holder and leveled with a glass slide.

Desolvation of an ethyl acetate/ethanol solvate, for example by air-drying, provides a solvent-free crystalline form of ABT-263 free base. PXRD peaks for Form I ABT-263 free base are listed in Table 1. A PXRD pattern having peaks substantially as indicated therein can be used to identify crystalline ABT-263 free base, more particularly Form I ABT-263 free base. The phrase “substantially as indicated” in the present context means having peaks that are not shifted more than about 0.2° 2θ from the indicated position.

TABLE 1
PXRD peak listing: solvent-free crystal polymorph Form
I ABT-263 free base
Peak Position (° 2θ)

6.21
6.72
9.66
10.92
11.34
12.17
14.28
16.40
16.95
17.81
18.03
18.47
19.32
20.10
21.87

Desolvation of most solvates, including 1-propanol, 2-propanol, methanol, benzene, toluene, dioxane/hexanes, methyl acetate/hexanes and chloroform solvates, provides a solvent-free crystalline form of ABT-263 free base that is shown by PXRD to be identical to the crystalline form produced by desolvation of the ethyl acetate/ethanol solvate.

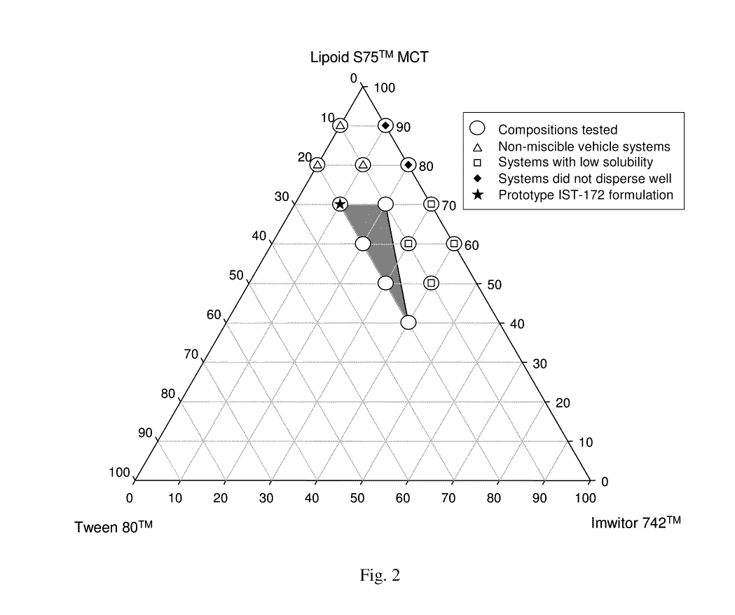

Desolvation of pyridine and anisole solvates provides a solvent-free crystalline form of ABT-263 free base that is shown by PXRD to be different from the form produced by desolvation of the ethyl acetate/ethanol solvate. The crystalline form derived from desolvation of the pyridine or anisole solvate is designated Form II. A PXRD scan of Form II ABT-263 free base is shown in FIG. 2. PXRD peaks for Form II ABT-263 free base are listed in Table 2. A PXRD pattern having peaks substantially as indicated therein can be used to identify crystalline ABT-263 free base, more particularly Form II ABT-263 free base.

TABLE 2
PXRD peak listing: solvent-free crystal polymorph Form II
ABT-263 free base
Peak Position (° 2θ)

5.79
8.60
9.34
10.79
11.36
11.59
12.76
13.23
13.73
14.01
14.72
15.00
16.28
17.07
17.48
18.75
19.34
19.71
20.56
21.35

PXRD peaks especially diagnostic for Form I ABT-263 free base, in particular for distinguishing Form I from Form II, include the peaks at 6.21, 6.72, 12.17, 18.03 and 20.10° 20, in each case ±0.2° 2θ. In one embodiment, Form I ABT-263 free base is characterized at least by a peak at any one or more of these positions. In another embodiment, Form I ABT-263 free base is characterized at least by a peak at each of these positions. In yet another embodiment, Form I ABT-263 free base is characterized by a peak at each of the positions shown in Table 1.

PXRD peaks especially diagnostic for Form II ABT-263 free base, in particular for distinguishing Form II from Form I, include the peaks at 5.79, 8.60, 12.76, 15.00 and 20.56° 2θ, in each case ±0.2° 2θ. In one embodiment, Form II ABT-263 free base is characterized at least by a peak at any one or more of these positions. In another embodiment, Form II ABT-263 free base is characterized at least by a peak at each of these positions. In yet another embodiment, Form II ABT-263 free base is characterized by a peak at each of the positions shown in Table 2.

Any of the crystalline forms of ABT-263 free base, including solvated forms, can be useful as API for preparation of a capsule of the present invention. However, solvent-free forms such as Form I and Form II are generally preferred for this purpose.

Without being bound by theory, it is believed that the therapeutic efficacy of compounds of Formula I is due at least in part to their ability to bind to a Bcl-2 family protein such as Bcl-2, Bcl-X_{L} or Bcl-w in a way that inhibits the anti-apoptotic action of the protein, for example by occupying the BH3 binding groove of the protein. It will generally be found desirable to select a compound having high binding affinity for a Bcl-2 family protein, for example a Kᵢ not greater than about 5 nM, preferably not greater than about 1 nM.

A composition as provided herein comprising any specific compound disclosed in the '135 publication is expressly contemplated as an embodiment of the present invention.

In a more particular embodiment, the composition comprises ABT-263 or a salt thereof. In a still more particular embodiment, the composition comprises ABT-263 free base or a salt, for example a bis-salt, thereof. In an even more particular embodiment, the composition comprises ABT-263 free base or ABT-263 bis-HCl.

Amounts, concentrations and dosages of a compound of Formula I or a salt thereof, for example of ABT-263 free base or ABT-263 bis-HCl, are expressed herein as free base equivalent, unless the context demands otherwise. Illustratively, in the case of ABT-263 bis-HCl, 1 mg free base equivalent translates to about 1.075 mg of the salt. Unless otherwise indicated, concentrations expressed as percentages herein are by weight.

A composition of the present invention contains a compound of Formula I or a salt thereof, for example ABT-263 free base or ABT-263 bis-HCl, in a free base equivalent amount of at least about 2.5% by weight. An active ingredient concentration in a liquid composition indicated herein to be 25 mg/l (a weight/volume concentration) will be understood to be “about 2.5% by weight” and at least in that regard within the scope of the present invention. An upper limit of concentration of a compound of Formula I or a salt thereof, for example ABT-263 free base or ABT-263 bis-HCl, in a composition is dictated by physical constraints such as drug solubility in the case of liquid solution compositions and by amounts of excipient ingredients required, e.g., for acceptable bioavailability, in the case of solid compositions, but is unlikely to exceed about 50% by weight.

In various embodiments, the free base equivalent concentration of the sole or first active ingredient in the composition is at least about 3%, at least about 4%, at least about 5% or at least about 10%, by weight, or at least about 30 mg/l, at least about 40 mg/l, at least about 50 mg/l or at least about 100 mg/l.

The sole or first active ingredient is present in the composition in an amount that can be therapeutically effective when the composition is administered to a subject in need thereof according to an appropriate regimen. Typically, a unit dose (the amount administered at a single time), which can be administered at an appropriate frequency, e.g., twice daily to once weekly, is about 10 to about 1,000 mg free base equivalent, depending on the compound in question. Where frequency of administration is once daily (q.d.), unit dose and daily dose are the same. Illustratively, for example where the drug is ABT-263, the unit dose is typically about 25 to about 1,000 mg, more typically about 50 to about 500 mg, for example about 50, about 100, about 150, about 200, about 250, about 300, about 350, about 400, about 450 or about 500 mg. Where the composition is provided as discrete dosage forms such as capsules or tablets, the unit dose can generally be delivered in one to a small plurality, most typically 1 to about 10, such dosage forms. The higher the unit dose, the more desirable it becomes to select a formulation with a relatively high concentration of the drug therein.

Necessarily where the sole or first active ingredient is in solution in a liquid carrier, and optionally where the sole or first active ingredient is in solid-state form as defined herein, the composition further comprises an antioxidant.

An “antioxidant” or compound having “antioxidant” properties is a chemical compound that prevents, inhibits, reduces or retards oxidation of another chemical or itself. Antioxidants can improve stability and shelf-life of a lipid formulation as described herein by, for example, preventing, inhibiting, reducing or retarding oxidation of the compound of Formula I in the formulation.

Enhancement of stability or shelf-life can be evaluated, for example, by monitoring rate of appearance or build-up of sulfoxides in the formulation. Sulfoxides in total can be monitored by repeated sampling and analysis; alternatively samples can be analyzed more specifically for the sulfoxide degradation product of the compound of Formula I, i.e., the compound having the formula

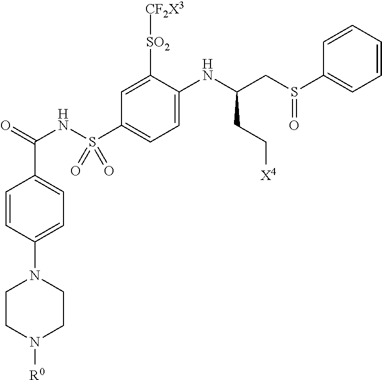

where X³, X⁴ and R⁰ are as indicated above; or the sulfoxide degradation product of ABT-263, having the formula

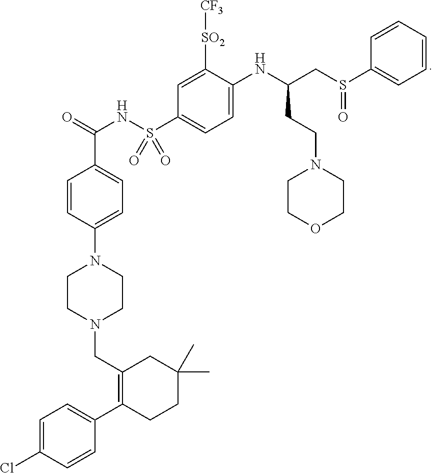

Reference herein to the sulfoxide degradation product will be understood to include both diastereomers at the sulfur atom stereocenter in the sulfoxide group.

An “antioxidant effective amount” of an antioxidant herein is an amount that provides
(a) a substantial reduction (for example a reduction of at least about 25%, at least about 50%, at least about 75%, at least about 80%, at least about 85% or at least about 90%) in the formation or accumulation of a degradation product, for example the sulfoxide degradation product above, and/or
(b) a substantial increase (for example at least about 30, at least about 60, at least about 90 or at least about 180 days) in the time taken for the degradation product to reach a threshold level,
   in a formulation containing the antioxidant, by comparison with an otherwise similar formulation containing no antioxidant. A storage-stability study to determine degree of (a) reduction in formation or accumulation of the degradation product or (b) increase in time taken for a degradation product to reach a threshold level in the formulation can be conducted at any appropriate temperature or range of temperatures. Illustratively, a study at about 5° C. can be indicative of storage stability under refrigerated conditions, a study at about 20-25° C. can be indicative of storage stability under typical ambient conditions, and a study at about 30° C. or higher temperature can be useful in an accelerated-aging study. Any appropriate threshold level of the degradation product can be selected as an end-point, for example in the range from about 0.2% to about 2% of the initial amount of the compound of Formula I present.

In various illustrative embodiments, the antioxidant is included in an amount effective to hold oxidative degradation of the drug

(a) below about 1% for at least about 3 months;

(b) below about 1% for at least about 6 months;

(c) below about 1% for at least about 1 year;

(d) below about 0.5% for at least about 3 months;

(e) below about 0.5% for at least about 6 months; or

(f) below about 0.5% for at least about 1 year;

in the formulation when stored under ambient conditions (e.g., about 20-25° C.) in a sealed container opaque to ultraviolet light, as measured for example by amount of the sulfoxide degradation product present at the end of the recited storage period.

Antioxidants used in pharmaceutical compositions are most typically agents that inhibit generation of oxidative species such as triplet or singlet oxygen, superoxides, peroxide and free hydroxyl radicals, or agents that scavenge such oxidative species as they are generated. Examples of commonly used antioxidants of these classes include butylated hydroxyanisole (BHA), butylated hydroxytoluene (BHT), retinyl palmitate, tocopherol, propyl gallate, ascorbic acid and ascorbyl palmitate. The present inventors have found, however, that at least some commonly used antioxidants are ineffective to protect ABT-263 from excessive sulfoxide formation in encapsulated liquid formulations as described herein.

For example, BHA, added at 0.2% by weight to a 15% by weight solution of ABT-263 free base in a medium referred to herein as “IPT-253” (20% Imwitor 742™, 50% Phosal 53 MCT™, 30% Tween™ 80), has been found to have no effect on sulfoxide formation in a 4-week stability study at 40° C. without nitrogen purging of headspace, as shown in Table 3. A full report of this study is found in Example 7 herein.

TABLE 3
Effect of 0.2% BHA on ABT-263 sulfoxide formation in IPT-253
solution

| | |
|---|---|
| Time | % Total sulfoxides |

| (weeks) | No antioxidant | 0.2% BHA |
|---|---|---|
| 0 | not detectable | 0.06 |
| 1 | 0.26 | 0.29 |
| 2 | 0.47 | 0.49 |
| 3 | 0.56 | 0.58 |
| 4 | 0.67 | 0.68 |

Antioxidants that, by contrast, have been found effective are heavier-chalcogen antioxidants (HCAs) that are believed, without being bound by theory, to function primarily as competitive substrates, i.e., as “sacrificial” antioxidants, which are preferentially attacked by oxidative species thereby protecting the drug from excessive degradation.

In some embodiments, the HCA comprises one or more antioxidant compounds of Formula II

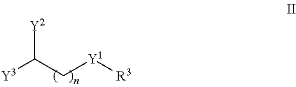

where

n is 0, 1 or 2;

Y¹ is S or Se;

Y² is NHR¹, OH or H, where R¹ is alkyl or alkylcarbonyl;

Y³ is COOR² or CH₂OH, where R² is H or alkyl; and

R³ is H or alkyl;

where alkyl groups are independently optionally substituted with one of more substituents independently selected from the group consisting of carboxyl, alkylcarbonyl, alkoxycarbonyl, amino and alkylcarbonylamino; a pharmaceutically acceptable salt thereof; or, where Y¹ is S and R³ is H, an —S—S— dimer thereof or pharmaceutically acceptable salt of such dimer.

In other embodiments, the HCA is an antioxidant compound of Formula III:

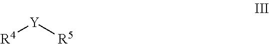

where
Y is S, Se or S—S; and
R⁴ and R⁵ are independently selected from H, alkyl and (CH₂)ₙR⁶ where n is 0-10 and R⁶ is arylcarbonyl, alkylcarbonyl, alkoxycarbonyl, carboxyl or CHR⁷R⁸-substituted alkyl, where R⁷ and R⁸ are independently CO₂R⁹, CH₂OH, hydrogen or NHR¹⁰, where R⁹ is H, alkyl, substituted alkyl or arylalkyl and R¹⁰ is hydrogen, alkyl, alkylcarbonyl or alkoxycarbonyl.

An “alkyl” substituent or an “alkyl” or “alkoxy” group forming part of a substituent according to Formula II or Formula III is one having 1 to about 18 carbon atoms and can consist of a straight or branched chain.

An “aryl” group forming part of a substituent according to Formula III is a phenyl group, unsubstituted or substituted with one or more hydroxy, alkoxy or alkyl groups.

In some embodiments, R¹ in Formula II is C₁₋₄ alkyl (e.g., methyl or ethyl) or (C₁₋₄ alkyl)carbonyl (e.g., acetyl).

In some embodiments, R² in Formula II is H or C₁₋₁₈ alkyl, for example methyl, ethyl, propyl (e.g., n-propyl or isopropyl), butyl (e.g., n-butyl, isobutyl or t-butyl), octyl (e.g., n-octyl or 2-ethylhexyl), dodecyl (e.g., lauryl), tridecyl, tetradecyl, hexadecyl or octadecyl (e.g., stearyl).

R³ is typically H or C₁₋₄ alkyl (e.g., methyl or ethyl).

The HCA can be, for example, a natural or synthetic amino acid or a derivative thereof such as an alkyl ester or N-acyl derivative, or a salt of such amino acid or derivative. Where the amino acid or derivative thereof is derived from a natural source it is typically in the L-configuration; however it is understood that D-isomers and D,L-isomer mixtures can be substituted if necessary.

Non-limiting examples of HCAs useful herein include β-alkylmercaptoketones, cysteine, cystine, homocysteine, methionine, thiodiglycolic acid, thiodipropionic acid, thioglycerol, selenocysteine, selenomethionine and salts, esters, amides and thioethers thereof; and combinations thereof. More particularly, one or more HCAs can be selected from N-acetylcysteine, N-acetylcysteine butyl ester, N-acetylcysteine dodecyl ester, N-acetyl-cysteine ethyl ester, N-acetylcysteine methyl ester, N-acetylcysteine octyl ester, N-acetyl-cysteine propyl ester, N-acetylcysteine stearyl ester, N-acetylcysteine tetradecyl ester, N-acetylcysteine tridecyl ester, N-acetylmethionine, N-acetylmethionine butyl ester, N-acetylmethionine dodecyl ester, N-acetylmethionine ethyl ester, N-acetylmethionine methyl ester, N-acetylmethionine octyl ester, N-acetylmethionine propyl ester, N-acetylmethionine stearyl ester, N-acetylmethionine tetradecyl ester, N-acetylmethionine tridecyl ester, N-acetyl-selenocysteine, N-acetylselenocysteine butyl ester, N-acetylselenocysteine dodecyl ester, N-acetylselenocysteine ethyl ester, N-acetylselenocysteine methyl ester, N-acetylseleno-cysteine octyl ester, N-acetylselenocysteine propyl ester, N-acetylselenocysteine stearyl ester, N-acetylselenocysteine tetradecyl ester, N-acetylselenocysteine tridecyl ester, N-acetylseleno-methionine, N-acetylselenomethionine butyl ester, N-acetylselenomethionine dodecyl ester, N-acetylselenomethionine ethyl ester, N-acetylselenomethionine methyl ester, N-acetyl-selenomethionine octyl ester, N-acetylselenomethionine propyl ester, N-acetylseleno-methionine stearyl ester, N-acetylselenomethionine tetradecyl ester, N-acetylseleno-methionine tridecyl ester, cysteine, cysteine butyl ester, cysteine dodecyl ester, cysteine ethyl ester, cysteine methyl ester, cysteine octyl ester, cysteine propyl ester, cysteine stearyl ester, cysteine tetradecyl ester, cysteine tridecyl ester, cystine, cystine dibutyl ester, cystine di(dodecyl) ester, cystine diethyl ester, cystine dimethyl ester, cystine dioctyl ester, cystine dipropyl ester, cystine distearyl ester, cystine di(tetradecyl) ester, cystine di(tridecyl) ester, N,N-diacetylcystine, N,N-diacetylcystine dibutyl ester, N,N-diacetylcystine diethyl ester, N,N-diacetylcystine di(dodecyl) ester, N,N-diacetylcystine dimethyl ester, N,N-diacetylcystine dioctyl ester, N,N-diacetylcystine dipropyl ester, N,N-diacetylcystine distearyl ester, N,N-diacetylcystine di(tetradecyl) ester, N,N-diacetylcystine di(tridecyl) ester, dibutyl thiodiglycolate, dibutyl thiodipropionate, di(dodecyl) thiodiglycolate, di(dodecyl) thiodipropionate, diethyl thiodiglycolate, diethyl thiodipropionate, dimethyl thiodiglycolate, dimethyl thiodipropionate, dioctyl thiodiglycolate, dioctyl thiodipropionate, dipropyl thiodiglycolate, dipropyl thiodipropionate, distearyl thiodiglycolate, distearyl thiodipropionate, di(tetradecyl) thiodiglycolate, di(tetradecyl) thiodipropionate, homocysteine, homocysteine butyl ester, homocysteine dodecyl ester, homocysteine ethyl ester, homocysteine methyl ester, homocysteine octyl ester, homocysteine propyl ester, homocysteine stearyl ester, homocysteine tetradecyl ester, homocysteine tridecyl ester, methionine, methionine butyl ester, methionine dodecyl ester, methionine ethyl ester, methionine methyl ester, methionine octyl ester, methionine propyl ester, methionine stearyl ester, methionine tetradecyl ester, methionine tridecyl ester, S-methylcysteine, S-methyl-cysteine butyl ester, S-methylcysteine dodecyl ester, S-methylcysteine ethyl ester, S-methyl-cysteine methyl ester, S-methylcysteine octyl ester, S-methylcysteine propyl ester, S-methyl-cysteine stearyl ester, S-methylcysteine tetradecyl ester, S-methylcysteine tridecyl ester, selenocysteine, selenocysteine butyl ester, selenocysteine dodecyl ester, selenocysteine ethyl ester, selenocysteine methyl ester, selenocysteine octyl ester, selenocysteine propyl ester, selenocysteine stearyl ester, selenocysteine tetradecyl ester, selenocysteine tridecyl ester, selenomethionine, selenomethionine butyl ester, selenomethionine dodecyl ester, seleno-methionine ethyl ester, selenomethionine methyl ester, selenomethionine octyl ester, seleno-methionine propyl ester, selenomethionine stearyl ester, selenomethionine tetradecyl ester, selenomethionine tridecyl ester, thiodiglycolic acid, thiodipropionic acid, thioglycerol, isomers and mixtures of isomers thereof, and salts thereof.

In some embodiments, the HCA selected is a sulfur-containing antioxidant.

Salts of HCA compounds can be acid addition salts such as the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts. In a particular embodiment, the hydrochloride salt of one of the compounds individually mentioned above is present in the composition in an antioxidant effective amount.

Without being bound by theory, it is generally believed that HCAs such as those exemplified above protect the active compound by being themselves more readily oxidizable and, therefore, being oxidized preferentially over the drug compound. In general, for this mode of operation to provide an acceptable degree of protection for the drug compound, an antioxidant of Formula II or Formula III must be present in a substantial amount, for example in a molar ratio to the drug compound of at least about 1:10. In some embodiments, the molar ratio of antioxidant to the drug compound is about 1:10 to about 2:1, for example about 1:5 to about 1.5:1. Best results will sometimes be obtained when the molar ratio is approximately 1:1, i.e., about 8:10 to about 10:8.

This typical requirement for a relatively high antioxidant concentration in the formulation places constraints both on the selection of antioxidant and on the selection of other formulation components, particularly in liquid solution compositions of the invention. For such compositions, a carrier system must be selected that is capable of dissolving not only the active agent but also the antioxidant, in an antioxidant effective amount. One of skill in the art can select a suitable lipid carrier, which can comprise a single lipid material or a mixture of two or more such materials, by routine solubility testing based on the disclosure herein.

Notwithstanding the antioxidant efficacy of HCAs of Formula II or Formula III, the present inventors have found that, at molar ratios of approximately 1:1, such antioxidants have a tendency to result in solutions that become cloudy upon storage, when ABT-263 is used in the form of its free base. For solutions containing ABT-263 in the form of its bis-HCl salt, this tendency is absent or at least less marked.

However, in yet another unexpected discovery, ABT-263 free base has been found to be less susceptible to sulfoxide formation than ABT-263 bis-HCl when formulated in lipid solution (but in the absence of antioxidant), as shown in Table 6 (see Example 3 hereinbelow). The solvent system in Solution A is Phosal 53 MCT™/ethanol, 9:1 v/v; and in Solution B is Labrafil M 1944 CS™/oleic acid/polysorbate 80, 30%/40%/30% by weight. (Labrafil M 1944 CS™ of Gattefossé contains polyoxyethylene glyceryl monooleate.) The three-week study was conducted at 40° C. without nitrogen purging of headspace.

To take advantage of the unexpected finding that ABT-263 is less susceptible to sulfoxide formation in its free base than salt form, the present inventors have turned to a different class of sulfur-containing antioxidants, namely inorganic antioxidants of the sulfite, bisulfite, metabisulfite and thiosulfate classes. To complicate matters, these antioxidants are poorly lipid-soluble and must be introduced to the carrier or drug-carrier system in aqueous solution. Presence of water promotes sulfoxide formation in ABT-263 solutions, the very effect that is sought to be minimized. To restrict the amount of added water, poorly lipid-soluble antioxidants are, in one embodiment of the present invention, added at much lower concentrations than those providing molar equivalence to the concentration of ABT-263.

Where a poorly lipid-soluble antioxidant such as a sulfite, bisulfite, metabisulfite or thiosulfate antioxidant is used, it is accompanied in the composition by water in an amount not exceeding about 1% by weight, for example about 0.2% to about 0.8% by weight. The amount of such antioxidant that can be introduced in such a small amount of water typically does not exceed about 0.2% by weight, and is for example an amount of about 0.02% to about 0.2%, or about 0.05% to about 0.15%, by weight, of the composition.

To minimize the amount of water added to the formulation, it is desirable to provide the antioxidant in the form of a relatively concentrated aqueous stock solution, for example having at least about 10% by weight antioxidant. However, it has been found that where an excessively concentrated stock solution (e.g., about 20% or higher) is used, this can result in undesirable precipitation of solids in the formulation. Suitable concentrations of antioxidant in the stock solution are typically about 10% to about 18%, illustratively about 15%, by weight.

Sodium and potassium salts of sulfites, bisulfites, metabisulfites and thiosulfates are useful antioxidants according to the present embodiment; more particularly sodium and potassium metabisulfites.

To further minimize sulfoxide formation, a chelating agent such as EDTA or a salt thereof (e.g., disodium EDTA or calcium disodium EDTA) is optionally added, for example in an amount of about 0.002% to about 0.02% by weight of the composition. EDTA can be added as an aqueous stock solution in the same manner as the antioxidant. The antioxidant and EDTA can, if desired, be added as components of the same stock solution. Chelating agents sequester metal ions that can promote oxidative degradation.

Surprisingly at the very low antioxidant concentrations contemplated herein (typically the molar ratio of poorly lipid-soluble antioxidant to ABT-263 according to the present embodiment is no greater than about 1:20), sulfoxide formation has been found to remain within acceptable limits, as illustrated in Example 12 herein.

Sulfoxide formation can be further minimized by selecting formulation ingredients having low peroxide value. Peroxide value is a well established property of pharmaceutical excipients and is generally expressed (as herein) in units corresponding to milliequivalents of peroxides per kilogram of excipient (meq/kg). Some excipients inherently have low peroxide value, but others, for example those having unsaturated fatty acid such as oleyl moieties and/or polyoxyethylene chains, can be sources of peroxides. In the case of polysorbate 80, for example, it is preferable to select a source of polysorbate 80 having a peroxide value not greater than about 5, for example not greater than about 2. Suitable sources include Crillet 4HP™ and Super-Refined Tween™ 80, both available from Croda.

### First Composition Embodiment

A composition of the first embodiment set forth hereinabove comprises (a) a compound of Formula I or a pharmaceutically acceptable salt thereof, in a free base equivalent amount of at least about 2.5% by weight of the composition; (b) a pharmaceutically acceptable HCA; and (c) a substantially non-aqueous pharmaceutically acceptable carrier that comprises one or more lipids; wherein said compound and the antioxidant are in solution in the carrier.

The term “drug-carrier system” as used in description of compositions of the present embodiment comprises a carrier having at least one drug homogeneously distributed therein. In such compositions the drug (a compound of Formula I or a salt thereof) and HCA are in solution in the carrier, and, in some of these compositions, the drug-carrier system constitutes essentially the entire composition. In other compositions, the drug-carrier system is encapsulated within a capsule shell that is suitable for oral administration; in such embodiments the composition comprises the drug-carrier system and the capsule shell.

A drug-carrier system of the present embodiment is typically liquid, but in some compositions the carrier and/or the drug-carrier system can be solid or semi-solid. For example, a drug-carrier system can illustratively be prepared by dissolving the drug and HCA in a carrier at a temperature above the melting or flow point of the carrier, and cooling the resulting solution to a temperature below the melting or flow point to provide a solid drug-carrier system. The drug-carrier system can optionally comprise a solid or semi-solid substrate having the drug solution adsorbed therein or thereon. Examples of such substrates include particulate diluents such as lactose, starches, silicon dioxide, etc., and polymers such as polyacrylates, high molecular weight PEGs, or cellulose derivatives, e.g., hydroxypropylmethylcellulose (HPMC). Where a solid solution is desired, a high melting point ingredient such as a wax can be included. A solid drug-carrier system can optionally be encapsulated or, if desired, delivered in tablet form. The drug-carrier system can, in some embodiments, be adsorbed on, or impregnated into, a drug delivery device.

In a composition of the present embodiment, the drug is “in solution” in the carrier. This will be understood to mean that substantially all of the drug is in solution, i.e., no substantial portion, for example no more than about 2%, or no more than about 1%, of the drug is in solid (e.g., crystalline) form, whether dispersed, for example in the form of a suspension, or not. In practical terms, this means that the drug must normally be formulated at a concentration below its limit of solubility in the carrier. It will be understood that the limit of solubility can be temperature-dependent, thus selection of a suitable concentration should take into account the range of temperatures to which the composition is likely to be exposed in normal storage, transport and use.

Not only the drug, but also the HCA, is “in solution” as defined above in the carrier. Where the HCA is poorly lipid-soluble and has to be introduced to the carrier or drug-carrier system in aqueous solution, a surfactant, more particularly a non-phospholipid surfactant, may be necessary to avoid phase separation.

The carrier according to the present embodiment is “substantially non-aqueous”, i.e., having no water, or having an amount of water that is small enough to be, in practical terms, essentially non-deleterious to performance or properties of the composition. Typically, the carrier comprises zero to less than about 5% by weight water. It will be understood that certain ingredients useful herein can bind small amounts of water on or within their molecules or supramolecular structures; such bound water if present does not affect the “substantially non-aqueous” character of the carrier as defined herein. Furthermore, as indicated above, use of a poorly lipid-soluble antioxidant requires that a small amount of water (not more than about 1% by weight of the drug-carrier system) be added; again, this does not affect the “substantially non-aqueous” character of the carrier as defined herein.

In some compositions, the carrier comprises one or more glyceride materials. Suitable glyceride materials include, without limitation, medium to long chain mono-, di- and triglycerides. The term “medium chain” herein refers to hydrocarbyl chains individually having no less than about 6 and less than about 12 carbon atoms, including for example C₈ to C₁₀ chains. Thus glyceride materials comprising caprylyl and capryl chains, e.g., caprylic/capric mono-, di- and/or triglycerides, are examples of “medium chain” glyceride materials herein. The term “long chain” herein refers to hydrocarbyl chains individually having at least about 12, for example about 12 to about 18, carbon atoms, including for example lauryl, myristyl, cetyl, stearyl, oleyl, linoleyl and linolenyl chains. Medium to long chain hydrocarbyl groups in the glyceride materials can be saturated, mono- or polyunsaturated.

In one embodiment the carrier comprises a medium chain and/or a long chain triglyceride material. A suitable example of a medium chain triglyceride material is a caprylic/capric triglyceride product such as Captex 355 EP™ of Abitec Corp. and products substantially equivalent thereto. Suitable examples of long chain triglycerides include any pharmaceutically acceptable vegetable oil, for example canola, coconut, corn, cottonseed, flaxseed, olive, palm, peanut, safflower, sesame, soy and sunflower oils, and mixtures of such oils. Oils of animal, particularly marine animal, origin can also be used, including for example fish oil.

A carrier system that has been found particularly useful in solubilizing both (a) a therapeutically effective amount of a compound of Formula I and (b) an antioxidant effective amount of an HCA, comprises two essential components: a phospholipid, and a pharmaceutically acceptable solubilizing agent for the phospholipid. It will be understood that reference in the singular to a (or the) phospholipid, solubilizing agent or other formulation ingredient herein includes the plural; thus combinations, for example mixtures, of more than one phospholipid, or more than one solubilizing agent, are expressly contemplated herein. The solubilizing agent, or the combination of solubilizing agent and phospholipid, also solubilizes the drug and the antioxidant, although other carrier ingredients, such as a surfactant or an alcohol such as ethanol, optionally present in the carrier can in some circumstances provide enhanced solubilization of the drug and antioxidant.

Any pharmaceutically acceptable phospholipid or mixture of phospholipids can be used. In general such phospholipids are phosphoric acid esters that yield on hydrolysis phosphoric acid, fatty acid(s), an alcohol and a nitrogenous base. Pharmaceutically acceptable phospholipids can include without limitation phosphatidylcholines, phosphatidylserines and phosphatidylethanolamines. In one embodiment the composition comprises phosphatidylcholine, derived for example from natural lecithin. Any source of lecithin can be used, including animal sources such as egg yolk, but plant sources are generally preferred. Soy is a particularly rich source of lecithin that can provide phosphatidylcholine for use in the present invention.

Illustratively, a suitable amount of phospholipid is about 15% to about 75%, for example about 30% to about 60%, by weight of the carrier, although greater and lesser amounts can be useful in particular situations.

Ingredients useful as components of the solubilizing agent are not particularly limited and will depend to some extent on the particular drug and HCA and the desired concentration of each and of phospholipid. In one embodiment, the solubilizing agent comprises one or more glycols, one or more glycolides and/or one or more glyceride materials.

Glycols are generally suitable only for non-encapsulated formulations or where a soft capsule shell is to be used, and tend to be incompatible with hard shells such as hard gelatin shells. Suitable glycols include propylene glycol and polyethylene glycols (PEGs) having molecular weight of about 200 to about 1,000 g/mol, e.g., PEG-400, which has an average molecular weight of about 400 g/mol. Such glycols can provide relatively high solubility of the drug; however the potential for oxidative degradation of the drug can be increased when in solution in a carrier comprising such glycols, for example because of the tendency of glycols to produce superoxides, peroxides and/or free hydroxyl radicals. The higher the glycol content of the carrier, the greater may be the tendency for degradation of a chemically unstable drug. In one embodiment, therefore, one or more glycols are present in a total glycol amount of at least about 1% but less than about 50%, for example less than about 30%, less than about 20%, less than about 15% or less than about 10% by weight of the carrier. In another embodiment, the carrier comprises substantially no glycol.

Glycolides are glycols such as propylene glycol or PEG esterified with one or more organic acids, for example medium- to long-chain fatty acids. Suitable examples include propylene glycol monocaprylate, propylene glycol monolaurate and propylene glycol dilaurate products such as, for example. Capmul PG8™, Capmul PG12™ and Capmul PG-2L™ respectively of Abitec Corp. and products substantially equivalent thereto.

Suitable glyceride materials for use together with a phospholipid include, without limitation, those mentioned above. Where one or more glyceride materials are present as a major component of the solubilizing agent, a suitable total amount of glycerides is an amount effective to solubilize the phospholipid and, in combination with other components of the carrier, effective to maintain the drug and antioxidant in solution. For example, glyceride materials such as medium chain and/or long chain mono-, di- and triglycerides, more typically medium-chain mono-, di- and triglycerides, can be present in a total glyceride amount of about 5% to about 70%, for example about 15% to about 60% or about 25% to about 50%, by weight of the carrier, although greater and lesser amounts can be useful in particular situations. In one embodiment, the encapsulated liquid comprises about 7% to about 30%, for example about 10% to about 25%, by weight medium-chain triglycerides and about 7% to about 30%, for example about 10% to about 25%, by weight medium-chain mono- and diglycerides.

Additional solubilizing agents that are other than glycols, glycolides or glyceride materials can be included if desired. Such agents, for example N-substituted amide solvents such as dimethylformamide (DMF) and N,N-dimethylacetamide (DMA), can, in specific cases, assist in raising the limit of solubility of the drug in the carrier, thereby permitting increased drug loading. However, the carriers useful herein generally provide adequate solubility of small-molecule drugs of interest herein without such additional agents.

Even when a sufficient amount of a glycol, glycolide or glyceride material is present to solubilize the phospholipid, the resulting carrier solution and/or the drug-carrier system may be rather viscous and difficult or inconvenient to handle. In such cases it may be found desirable to include in the carrier a viscosity reducing agent in an amount effective to provide acceptably low viscosity. An example of such an agent is an alcohol, more particularly ethanol, which is preferably introduced in a form that is substantially free of water, for example 99% ethanol, dehydrated alcohol USP or absolute ethanol. Excessively high concentrations of ethanol should, however, generally be avoided. This is particularly true where, for example, the drug-carrier system is to be administered in a gelatin capsule, because of the tendency of high ethanol concentrations to result in mechanical failure of the capsule. In general, suitable amounts of ethanol are 0% to about 25%, for example about 1% to about 20% or about 3% to about 15%, by weight of the carrier. Glycols such as propylene glycol or PEG and medium-chain mono- and diglycerides (for example caprylic/capric mono- and diglycerides) can also be helpful to lower viscosity; where the drug-carrier system is to be encapsulated in a hard capsule such as a hard gelatin capsule, medium-chain mono- and diglycerides are particularly useful in this regard.

Optionally, the carrier further comprises a pharmaceutically acceptable non-phospholipid surfactant. One of skill in the art will be able to select a suitable surfactant for use in a composition of the present embodiment, based on information herein. Such a surfactant can serve various functions, including for example enhancing dispersion of the encapsulated liquid upon release from the capsule in the aqueous environment of the gastrointestinal tract. Thus in one embodiment the non-phospholipid surfactant is a dispersing and/or emulsifying agent that enhances dispersion and/or emulsification of the capsule contents in real or simulated gastrointestinal fluid. Illustratively, a surfactant such as a polysorbate (polyoxyethylene sorbitan ester), e.g., polysorbate 80 (available for example as Tween 80™ from Uniqema), can be included in an amount of 0% to about 30%, for example about 7% to about 30% or about 10% to about 25%, by weight of the carrier. In some embodiments such a surfactant is included in an amount of 0% to about 5%, for example 0% to about 2% or 0% to about 1%, by weight of the carrier.

Conveniently, pre-blended products are available containing a suitable phospholipid+solubilizing agent combination for use in compositions of the present invention. Pre-blended phospholipid+solubilizing agent products can be advantageous in improving ease of preparation of the present compositions.

An illustrative example of a pre-blended phospholipid+solubilizing agent product is Phosal 50 PG™, available from Phospholipid GmbH, Germany, which comprises, by weight, not less than 50% phosphatidylcholine, not more than 6% lysophosphatidylcholine, about 35% propylene glycol, about 3% mono- and diglycerides from sunflower oil, about 2% soy fatty acids, about 2% ethanol, and about 0.2% ascorbyl palmitate.

Another illustrative example is Phosal 53 MCT™, also available from Phospholipid GmbH, which contains, by weight, not less than 53% phosphatidylcholine, not more than 6% lysophosphatidylcholine, about 29% medium chain triglycerides, 3-6% (typically about 5%) ethanol, about 3% mono- and diglycerides from sunflower oil, about 2% oleic acid, and about 0.2% ascorbyl palmitate (reference composition). A product having the above or substantially equivalent composition, whether sold under the Phosal 53 MCT™ brand or otherwise, is generically referred to herein as “phosphatidylcholine+medium chain triglycerides 53/29”. A product having “substantially equivalent composition” in the present context means having a composition sufficiently similar to the reference composition in its ingredient list and relative amounts of ingredients to exhibit no practical difference in properties with respect to utilization of the product herein.

Yet another illustrative example is Lipoid S75™, available from Lipoid GmbH, which contains, by weight, not less than 70% phosphatidylcholine in a solubilizing system. This can be further blended with medium-chain triglycerides, for example in a 30/70 weight/weight mixture, to provide a product (“Lipoid S75™ MCT”) containing, by weight, not less than 20% phosphatidylcholine, 2-4% phosphatidylethanolamine, not more than 1.5% lysophosphatidylcholine, and 67-73% medium-chain triglycerides.

Yet another illustrative example is Phosal 50 SA+™, available from Phospholipid GmbH, which contains, by weight, not less than 50% phosphatidylcholine and not more than 6% lysophosphatidylcholine in a solubilizing system comprising safflower oil and other ingredients.

The phosphatidylcholine component of each of these pre-blended products is derived from soy lecithin. Products of substantially equivalent composition may be obtainable from other suppliers.

A pre-blended product such as Phosal 50 PG™, Phosal 53 MCT™, Lipoid S75™ MCT or Phosal 50 SA+™ can, in some embodiments, constitute substantially the entire carrier system (other than the HCA as provided herein). In other embodiments, additional ingredients are present, for example medium-chain mono- and/or diglycerides, ethanol (additional to any that may be present in the pre-blended product), a non-phospholipid surfactant such as polysorbate 80, polyethylene glycol and/or other ingredients. Such additional ingredients, if present, are typically included in only minor amounts. Illustratively, phosphatidylcholine+medium chain triglycerides 53/29 can be included in the carrier in an amount of about 50% to 100%, for example about 80% to 100%, by weight of the carrier.

Some pre-blended products, including Phosal 50 PG™ and Phosal 53 MCT™, contain a small amount of ascorbyl palmitate, an antioxidant which does not meet the definition of an HCA herein. Presence of ascorbyl palmitate or other non-HCA is generally not detrimental, but if desired a pre-blended product without such antioxidant can be used as the carrier herein.

In some compositions of the present embodiment, the drug-carrier system is dispersible in an aqueous phase to form a non-gelling, substantially non-transparent liquid dispersion. This property can readily be tested by one of skill in the art, for example by adding 1 part of the drug-carrier system to about 20 parts of water with agitation at ambient temperature and assessing gelling behavior and transparency of the resulting dispersion. Compositions having ingredients in relative amounts as indicated herein will generally be found to pass such a test, i.e., to form a liquid dispersion that does not gel and is substantially non-transparent. In “non-gelling” embodiments, the composition does not contain a gel-promoting agent in a gel-promoting effective amount. If gelling behavior is desired, such an agent can be added. A “substantially non-transparent” dispersion is believed to be formed on mixing with an aqueous phase a composition of the invention having any substantial amount of the phospholipid component. However, for clarification it is emphasized that compositions of the invention themselves, being substantially non-aqueous, are generally clear and transparent. In this regard, it is noted that phospholipids tend to form bi- and multilamellar aggregates when placed in an aqueous environment, such aggregates generally being large enough to scatter transmitted light and thereby provide a non-transparent, e.g., cloudy, dispersion. In the case of phosphatidylcholine+medium chain triglycerides 53/29, for example, dispersion in an aqueous environment typically forms not only multilamellar aggregates but also a coarse oil-in-water emulsion. Presence of multilamellar aggregates can often be confirmed by microscopic examination in presence of polarized light, such aggregates tending to exhibit birefringence, for example generating a characteristic “Maltese cross” pattern.

Without being bound by theory, it is believed that behavior of the drug-carrier system of a composition of the invention upon mixing with an aqueous phase is indicative of how the composition interacts with gastrointestinal fluid following oral administration to a subject. Although formation of a gel can be useful for controlled-release topical delivery of a drug, it is believed that gelling would be detrimental to efficient gastrointestinal absorption. For this reason, embodiments of the invention described above, wherein the drug-carrier system does not gel when mixed with an aqueous phase, are generally preferred. It is further believed, again without being bound by theory, that formation of bi- and multilamellar aggregates in the gastrointestinal fluid, as evidenced by non-transparency of the dispersion formed upon mixing the drug-carrier system with an aqueous phase, can be an important factor in providing the relatively high bioavailability of certain compositions of the invention when administered orally.

Carrier ingredients and amounts thereof are selected to provide solubility of the drug in the carrier of at least about 25 mg/ml at about 25° C.

Illustratively, a drug-carrier system according to the present embodiment comprises:

about 5% to about 20% by weight ABT-263 free base,

about 15% to about 60% by weight phosphatidylcholine,

about 7% to about 30% by weight medium-chain triglycerides,

about 7% to about 30% by weight medium-chain mono- and diglycerides,

about 7% to about 30% polysorbate 80 surfactant,

about 0.02% to about 0.2% by weight sodium or potassium metabisulfite,

about 0.003% to about 0.01% by weight EDTA or salt thereof, and

about 0.2% to about 0.8% by weight water.

Other excipients can optionally be present in the formulation, so long as they do not adversely affect the storage stability, safety or therapeutic efficacy of the formulation to an unacceptable degree. However, in a more particular embodiment, the drug-carrier system consists essentially of the ingredients listed immediately above.

A prototype formulation of the present embodiment comprises a size 0 hard gelatin capsule shell having encapsulated therewithin a liquid solution that comprises:

about 11% by weight ABT-263 free base,

about 33% by weight phosphatidylcholine,

about 16% by weight medium-chain triglycerides,

about 20% by weight medium-chain mono- and diglycerides,

about 20% by weight polysorbate 80 surfactant,

about 0.05% by weight sodium or potassium metabisulfite,

about 0.005% by weight EDTA or salt thereof, and

about 0.5% by weight water.

The term “about” in descriptions of prototype compositions herein will be understood to mean that the amounts shown can vary at least within usual manufacturing tolerances accepted in the pharmaceutical industry. Percentages may not add exactly to 100 because of rounding.

The present invention is not limited by the process used to prepare a composition as embraced or described herein. Any suitable process of pharmacy can be used. Illustratively, compositions of the present embodiment can be prepared by a process comprising simple mixing of the recited ingredients, wherein order of addition is not critical, to form a drug-carrier system. It is noted, however, that if a phospholipid component is used in its solid state, for example in the form of soy lecithin, it will generally be desirable to first solubilize the phospholipid with the solubilizing agent component or part thereof. Thereafter other ingredients of the carrier, if any, the drug and the antioxidant can be added by simple mixing, with agitation as appropriate. As mentioned above, use of a pre-blended product comprising phospholipid and solubilizing agent can simplify preparation of the composition. Optionally, the drug-carrier system can be used as a premix for capsule filling. The term “filling” used in relation to a capsule herein means placement of a desired amount of a composition in a capsule shell, and should not be taken to mean that all space in the capsule is necessarily occupied by the composition.

Where the drug-carrier system comprises a poorly lipid-soluble sulfur-containing antioxidant such as sodium or potassium metabisulfite, the process should be adjusted. An illustrative process for preparing such a drug-carrier system comprises the following steps.

An API that consists essentially of a compound of Formula I or a salt thereof, for example ABT-263 free base or ABT-263 bis-HCl, is dissolved in a medium comprising the phospholipid and at least a portion of the solubilizing agent to provide a lipid solution of the API. As noted above, a pre-blended product comprising the phospholipid and solubilizing agent can be used as the medium for dissolution of the API.

Where ABT-263 is to be formulated in its free base form, any solid-state form of ABT-263 free base can serve as the API. However, it will generally be found preferable to use a crystalline form of ABT-263 free base as API, for example a solvated or non-solvated crystalline form. In a particular embodiment of the present process, a non-solvated crystalline form such as Form I or Form II crystalline ABT-263 as described herein is used as API.

A non-phospholipid surfactant and, optionally, the balance of the solubilizing agent, is admixed with the solubilizing agent (prior to or simultaneously with dissolution of the API) or with the lipid solution (after dissolution of the API). As noted above, the non-phospholipid surfactant is illustratively a polysorbate such as polysorbate 80. The balance of the solubilizing agent can be the same material as the portion of solubilizing agent used together with the phospholipid to dissolve the API; alternatively it can be a different material. For example, the portion of solubilizing agent used together with the phospholipid for dissolution of the API can comprise one or more medium-chain triglycerides, and the balance of solubilizing agent admixed in the present step can comprise one or more medium-chain mono- and/or diglycerides, for example a caprylic/capric mono- and diglyceride product such as Imwitor 742™.

Separately, a poorly lipid-soluble sulfur-containing antioxidant is dissolved in water to prepare an aqueous stock solution. Stock solutions at about 10% to about 18% by weight concentration will generally be found suitable, as explained above.

The aqueous stock solution is then admixed with the lipid solution, typically after addition of the non-phospholipid surfactant, to provide a liquid solution for encapsulation.

Optionally, the resulting liquid solution is encapsulated in a capsule shell by any known encapsulation process.

### Second Composition Embodiment

A composition of the second embodiment set forth hereinabove comprises a capsule shell having encapsulated therewithin, in an amount not greater than about 1000 mg per capsule, a liquid solution of a compound of Formula I or a pharmaceutically acceptable salt thereof in a free base equivalent amount of at least about 2.5% by weight of the solution, in a substantially non-ethanolic carrier that comprises as pharmaceutically acceptable excipients:
(a) at least one phospholipid,
(b) at least one solubilizing agent for the at least one phospholipid, selected from the group consisting of glycols, glycolides, glycerides and mixtures thereof,
(c) at least one non-phospholipid surfactant, and
(d) a pharmaceutically acceptable HCA.

In a capsule of the present embodiment, ABT-263 is “in solution” in the encapsulated liquid as in a composition of the first embodiment described above. The encapsulated liquid is “substantially non-ethanolic”, i.e., having no ethanol, or having an amount of ethanol that is small enough to be, in practical terms, essentially non-deleterious to performance or properties of the capsule. More particularly, any ethanol that is present must be below a threshold concentration at which integrity of the capsule shell is compromised. Typically the encapsulated liquid comprises zero to less than about 5% by weight ethanol. This is especially important where a hard capsule shell, for example a hard gelatin or hydroxypropylmethylcellulose (HPMC) capsule shell, is used. Soft capsule shells, for example soft gelatin or starch-based shells containing a plasticizer, can tolerate somewhat higher amounts of ethanol. Certain pre-blended phospholipid products useful herein contain small amounts of ethanol that are non-deleterious even to a hard gelatin capsule; for example Phosal 53 MCT™ can contain up to about 6% ethanol. When used illustratively in an amount not exceeding about 75% by weight of the encapsulated liquid, Phosal 53 MCT™ is seen to contribute ethanol in an amount not exceeding about 4.5% by weight of the encapsulated liquid, which remains “substantially non-ethanolic” as defined herein.

In most embodiments, the encapsulated liquid is also “substantially non-aqueous”, as defined above in relation to compositions of the first embodiment.

As indicated above, the encapsulated liquid comprises, inter alia, a phospholipid, and a pharmaceutically acceptable solubilizing agent for the phospholipid. The solubilizing agent, or the combination of solubilizing agent and phospholipid, may also assist in solubilizing the ABT-263, as may other ingredients, such as a non-phospholipid surfactant. Phospholipids and solubilizing agents, including pre-blended products, useful herein are as described above in relation to compositions of the first embodiment.

Illustratively, a suitable amount of phospholipid in the encapsulated liquid of the present embodiment is about 15% to about 60%, for example about 20% to about 45%, by weight of the encapsulated liquid, although greater and lesser amounts can be useful in particular situations.

If the solubilizing agent comprises one or more glycols, these can illustratively present in a total glycol amount of at least about 1% but less than about 50%, for example less than about 30%, less than about 20%, less than about 15% or less than about 10% by weight of the carrier. In some embodiments, the carrier comprises substantially no glycol.

Where one or more glycerides are present as a major component of the solubilizing agent, a suitable total amount of glycerides is an amount effective to solubilize the phospholipid and, in combination with other excipients, effective to maintain the compound of Formula I or salt thereof, for example ABT-263 free base or ABT-263 bis-HCl, in solution. For example, glycerides such as medium-chain mono-, di- and triglycerides can be present in a total glyceride amount of about 15% to about 60%, for example about 20% to about 45%, by weight of the encapsulated liquid, although greater and lesser amounts can be useful in particular situations. In one embodiment, the encapsulated liquid comprises about 7% to about 30%, for example about 10% to about 25%, by weight medium-chain triglycerides and about 7% to about 30%, for example about 10% to about 25%, by weight medium-chain mono- and diglycerides.

The encapsulated liquid of the present embodiment further comprises a pharmaceutically acceptable non-phospholipid surfactant, for example as described above in relation to compositions of the first embodiment. Illustratively, a surfactant such as a polysorbate, e.g., polysorbate 80, can be included in an amount of about 7% to about 30%, for example about 10% to about 25%, by weight of the encapsulated liquid.

Illustratively, the encapsulated liquid solution according to the present embodiment comprises:

about 5% to about 20% by weight ABT-263 free base,

about 15% to about 60% by weight phosphatidylcholine,

about 7% to about 30% by weight medium-chain triglycerides,

about 7% to about 30% by weight medium-chain mono- and diglycerides,

about 7% to about 30% polysorbate 80 surfactant,

about 0.02% to about 0.2% by weight sodium or potassium metabisulfite,

about 0.003% to about 0.01% by weight EDTA or salt thereof, and

about 0.2% to about 0.8% by weight water.

Other excipients can optionally be present in the encapsulated solution, so long as they do not adversely affect the storage stability, safety or therapeutic efficacy of the capsule to an unacceptable degree. However, in a more particular embodiment, the encapsulated liquid solution consists essentially of the ingredients listed immediately above.

The capsule shell can be of any pharmaceutically acceptable material, including hard or soft gelatin. A capsule shell size is selected appropriate to the amount of liquid to be encapsulated. For example, a size 0 capsule shell can be used to encapsulate up to about 600 mg of liquid and a size 00 capsule shell up to about 900 mg of liquid.

A prototype capsule of the present invention comprises a size 0 hard gelatin capsule shell having encapsulated therewithin a liquid solution that comprises:

about 50 mg ABT-263 free base,

about 150 mg phosphatidylcholine,

about 75 mg medium-chain triglycerides,

about 90 mg medium-chain mono- and diglycerides,

about 90 mg polysorbate 80 surfactant,

about 0.25 mg sodium or potassium metabisulfite,

about 0.025 mg EDTA or salt thereof, and

about 2.5 mg water.

Illustratively, a capsule of the invention can be prepared by a process comprising simple mixing of the recited ingredients, wherein order of addition is not critical, to form a liquid solution for encapsulation, followed by encapsulation of the liquid in a hard or soft gelatin capsule shell to form a capsule. It is noted, however, that if the phospholipid is used in its solid state, for example in the form of soy lecithin, it will generally be desirable to first solubilize the phospholipid with the solubilizing agent or part thereof. Thereafter other excipients and the ABT-263 can be added by simple mixing, with agitation as appropriate. Use of a pre-blended product comprising phospholipid and solubilizing agent can simplify preparation of the composition. For example, the phospholipid can comprise phosphatidylcholine and the solubilizing agent pre-blended therewith can comprise medium-chain triglycerides, as in the case of Phosal 53 MCT™ or Lipoid S75™ MCT. Illustratively, the pre-blended product comprises about 50% to about 75% phosphatidylcholine and about 15% to about 30% medium-chain triglycerides.

Where the solution for encapsulation comprises a poorly lipid-soluble sulfur-containing antioxidant such as sodium or potassium metabisulfite, the process should be adjusted. An illustrative process for preparing such a solution is as described above in relation to a composition of the first embodiment. The resulting liquid solution is then encapsulated in a capsule shell by any known encapsulation process.

### Third Composition Embodiment

A composition of the third embodiment set forth hereinabove comprises an orally deliverable liquid pharmaceutical composition comprising an aqueous medium having suspended therein a solid particulate compound having a D₉₀ particle size not greater than about 3 μm; wherein the compound is of Formula I or a pharmaceutically acceptable salt thereof, for example ABT-263 free base or ABT-263 bis-HCl, and is present in a free base equivalent amount of at least about 2.5% by weight of the composition; and wherein the aqueous medium further comprises at least one pharmaceutically acceptable surfactant and at least one pharmaceutically acceptable basifying agent in amounts that are effective together to inhibit particle size increase.

A suspension composition in accordance with the present embodiment comprises a nanosized solid particulate drug compound. It is found that in the suspensions described herein the drug nanoparticles do not appreciably agglomerate, resulting in production of stable formulations.

Unless the context demands otherwise, the term “nanoparticle” as used herein means a particle of size (i.e., diameter in the longest dimension of the particle) not greater than about 3 μm (3,000 nm). “Nanoparticles” as recited herein therefore include not only “submicron” particles, i.e., having a size less than about 1 μm, but also “micron-sized” particles of about 1 to about 3 μm. Likewise, the adjective “nanosized” as used herein refers to nanoparticles as defined immediately above. Unless the context demands otherwise, the term “nanoparticulate” as applied to a suspension or other composition herein, and likewise the term “nanosuspension”, means having a D₉₀ particle size not greater than about 3 μm.

The D₉₀ particle size of a composition is a parameter such that 90% by volume of particles in the composition are smaller in their longest dimension than that parameter, as measured by any conventional particle size measuring technique known to those skilled in the art. Such techniques include, for example, sedimentation field flow fractionation, photon correlation spectroscopy, light scattering, and disk centrifugation. In various compositions of the present embodiment, suspensions are provided having a D₉₀ particle size not greater than about 3,000 nm, not greater than about 2,000 nm, not greater than about 1,500 nm, not greater than about 1,000 nm, not greater than about 900 nm, not greater than about 800 nm, not greater than about 700 nm, not greater than about 600 nm or not greater than about 500 nm.

The D₅₀ particle size of a composition is a parameter such that 50% by volume of particles in the composition are smaller in their longest dimension than that parameter, as measured by any conventional particle size measuring technique known to those skilled in the art. D₅₀ particle size is therefore a measure of volume median particle size but is sometimes referred to as “average” or “mean” particle size. In various compositions of the present embodiment, suspensions are provided having a D₅₀ particle size not greater than about 1,000 nm, not greater than about 900 nm, not greater than about 800 nm, not greater than about 700 nm, not greater than about 600 nm, not greater than about 500 nm, not greater than about 400 nm, not greater than about 350 nm or not greater than about 300 nm.

In some cases, a suspension as provided herein has a D₉₀ particle size not greater than about 1,000 nm and a D₅₀ particle size not greater than about 400 nm. In other cases, a suspension as provided herein has a D₉₀ particle size not greater than about 800 nm and a D₅₀ particle size not greater than about 350 nm.

The terms “low solubility” and “poorly soluble” as used in relation to compositions of the present embodiment refer to a solubility in water not greater than about 100 μg/ml. The present invention can be especially advantageous for drugs that are essentially insoluble in water, i.e., having a solubility of less than about 10 μg/ml. It is believed, without being bound by theory, that the advantages of nanoparticulate suspensions for such drugs arise in part not only from improved dissolution rate, which is proportional to surface area according to the well known Whitney-Noyes equation, but also from improved solubility according to the Kelvin equation. This can result in enhanced bioavailability as well as potentially reduce food effect.

The nanoparticulate suspension comprises a compound of Formula I or a salt thereof as a discrete solid-state phase that can be crystalline, semi-crystalline or amorphous. In the case of ABT-263, the free base form of which, as prepared according to the '135 publication, is an amorphous or glassy solid, it is generally preferred to use a crystalline salt form of the drug, such as for example ABT-263 bis-HCl, in preparing the nanosuspension. However, upon suspension of the salt in presence of a basifying agent such as sodium bicarbonate, some conversion of salt to free base can occur, resulting in the solid-state phase becoming at least partly amorphous. Accordingly, in one embodiment, the nanosuspension comprises ABT-263 free base, ABT-263 bis-HCl or a combination thereof. Despite the likelihood that the drug particles in an ABT-263 nanosuspension are at least partly amorphous, a remarkably high degree of physical stability has been observed in such a nanosuspension, as illustrated in Example 14 below.

It has been found that nanoparticulate suspensions as described herein offer not only the advantage of physical stability providing acceptable product shelf life, but also the robustness of manufacturing process that is desirable for a commercial product.

The concentration of drug in the suspension is at least about 25 mg/ml, e.g., about 25 to about 500 mg/ml. Illustratively, for example where the drug is ABT-263, the drug concentration in various embodiments is about 25 to about 400 mg/ml, for example about 25, about 30, about 40, about 50, about 75, about 100, about 125, about 150 or about 200 mg/ml, by free base equivalent weight.

Compositions of the present invention have good storage-stability properties. In particular, they are physically stable, at least in that they do not have an unacceptable tendency to undergo particle size increase over time, for example through particle agglomeration. Particle agglomeration is a common problem in nanoparticulate suspensions. Surface modifying agents such as surfactants are important in reducing the tendency of nanoparticles to agglomerate; the at least one surfactant present in a composition of the present invention is believed, without being bound by theory, to help in this regard.

A “basifying agent” herein is any agent that raises the pH of the suspension medium. Any pharmaceutically acceptable basifying agent can be used, including without limitation hydroxides and bicarbonates of alkali metals such as sodium and potassium. The invention is illustrated herein with particular reference to sodium bicarbonate, but it will be recognized that other basifying agents can be substituted for sodium bicarbonate if desired.

Amount of sodium bicarbonate useful in a composition of the invention is not narrowly critical, and one of ordinary skill in the art can readily optimize the amount for any particular composition, for example by routine storage-stability testing. In general, good results can be obtained with sodium bicarbonate in an amount of about 20 to about 200 mg/ml, for example about 40 to about 160 mg/ml.

The choice and amount of surfactant is likewise not narrowly critical, and is likely to depend to some extent on the particular drug compound to be formulated and the drug loading desired. Non-limiting examples of surfactants include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers), for example poloxamer 188 and poloxamer 237; polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters, for example sorbitan monolaurate, sorbitan monooleate, sorbitan monopalmitate and sorbitan monostearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; α-tocopheryl polyethylene glycol succinate (TPGS); tyloxapol; and the like. In one embodiment, the at least one surfactant is a poloxamer or mixture of poloxamers. Poloxamer 188 is a specific example. One or more surfactants typically constitute in total about 10 to about 100 mg/ml of the suspension. In the case of poloxamer 188, an illustratively suitable amount is about 10 to about 100 mg/ml, for example about 15 to about 60 mg/ml, of the suspension.

The aqueous medium of the suspension can take the form of water, an aqueous injectable fluid such as saline (e.g., phosphate-buffered saline or PBS) or an imbibable liquid such as fruit juice or a carbonated beverage. In one embodiment the nanoparticulate drug compound, the at least one surfactant and at least one basifying agent (and optionally additional ingredients) are prepared as a dry powder mix for reconstitution with a suitable aqueous medium to form a suspension composition of the invention shortly before use. Such a reconstitutable powder should contain, in addition to the ingredients recited above, at least one pharmaceutically acceptable dispersant or bulking agent, typically a water-soluble material such as a sugar, e.g., dextrose, mannitol or dextran; a phosphate salt, e.g., sodium or potassium phosphate; an organic acid, e.g., citric acid or tartaric acid, or a salt thereof; or a mixture of such materials. A dry powder mix can alternatively be administered to a subject for resuspension of the nanoparticles in the gastrointestinal fluid; for such administration the powder mix can if desired be formed into a tablet or filled into a capsule.

It is desirable to provide a formulation that is not only physically stable but also chemically stable. More particularly, such a formulation should not exhibit an unacceptable degree of oxidative degradation of the compound of Formula I, for example at the thioether linkage of the (phenylsulfanyl)methyl group thereof.

In this regard, a composition of the present invention containing a compound of Formula I such as ABT-263 free base, ABT-263 bis-HCl or a combination thereof possesses a significant advantage over solution compositions of ABT-263 previously disclosed in the art, for example in the '135 publication or in Tse et al. (2008), supra. The solid-state form (whether crystalline, semi-crystalline or amorphous) of ABT-263 present in a nanosuspension as provided herein is believed to be significantly more resistant to oxidative degradation than ABT-263 in solution.

However, if desired, any remaining tendency for oxidative degradation can be further reduced by inclusion of a suitable antioxidant, more particularly an HCA as described hereinabove in the suspension composition.

In view of the aqueous nature of the suspension medium, water-soluble inorganic antioxidants of the sulfite, bisulfite, metabisulfite and thiosulfate classes can be particularly useful. Such antioxidants can be included in any suitable amount, for example about 0.02% to about 2%, or about 0.05% to about 1%, by weight, of the composition.

Sodium and potassium salts of sulfites, bisulfites, metabisulfites and thiosulfates are especially useful antioxidants according to the present embodiment; more particularly sodium and potassium metabisulfites.

To further minimize sulfoxide formation, a chelating agent such as EDTA or a salt thereof (e.g., disodium EDTA or calcium disodium EDTA) is optionally added, for example in an amount of about 0.002% to about 0.2% by weight of the composition.

Other optional ingredients of the suspension composition include buffers, coloring agents, flavoring agents, preservatives, sweeteners, tonicifying agents and combinations thereof.

A process for preparing a nanoparticulate pharmaceutical composition of the present embodiment comprises providing an API that comprises a compound of Formula I or a pharmaceutically acceptable salt thereof, for example ABT-263 or a crystalline salt thereof; wet-milling the API in presence of at least one basifying agent, such as sodium bicarbonate, to a D₉₀ particle size not greater than about 3 μm to provide a milled drug substance; and suspending the milled drug substance in an aqueous medium with the aid of at least one surfactant; wherein the at least one basifying agent and the at least one surfactant are present in the resulting suspension in amounts that are effective together to inhibit particle size increase.

Any suitable wet-milling process can be used. A particular wet-milling process that has been found useful is high-pressure homogenization as illustratively described in Example 13 below.

The present invention is not limited to compositions prepared by any process described herein; however, a composition prepared by the above process is a particular embodiment of the invention.

In one embodiment, the process further comprises adding at least one pharmaceutically acceptable dispersant or bulking agent to the suspension, drying (for example freeze-drying or lyophilizing, or alternatively spray-drying) the suspension to provide a reconstitutable dry powder, and optionally forming the powder into a tablet (for example by molding or compression) or filling the powder into a capsule, to prepare a unit dosage form.

In addition to the stabilizing benefits of sodium bicarbonate, it is found that in presence of sodium bicarbonate, wet-milling to smaller particle sizes, for example to a D₉₀ particle size not greater than about 700 nm, is possible. Without sodium bicarbonate, as illustratively shown in Example 14 hereinbelow, using the same processing parameters, D₉₀ particle size can not be reduced below about 1,000 nm. The wet-milling method used in the present process has the advantage, by comparison with dry-milling, that it reduces exposure of the API to high temperature and thereby reduces risk of thermal decomposition of the API. In one embodiment, processing temperature is controlled, for example within about 1 to about 5 degrees of a target temperature of about 5° C. to about 30° C. This can be achieved by conventional means, such as by running the formulation through a heat exchanger immersed in a chilled water bath.

The composition can be prepared for wet-milling at its final concentration, or it can be prepared at higher concentration and diluted to a desired concentration after wet-milling. The at least one surfactant and, if desired, optional additional ingredients, can be added before or after wet-milling.

### Fourth Composition Embodiment

A composition of the fourth embodiment set forth hereinabove comprises an orally deliverable solid dispersion comprising, in essentially non-crystalline, for example amorphous, form, a compound of Formula I or a pharmaceutically acceptable salt thereof in a free base equivalent amount of at least about 2.5% by weight of the composition, dispersed in a solid matrix that comprises (a) a pharmaceutically acceptable water-soluble polymeric carrier and (b) a pharmaceutically acceptable surfactant.

A solid dispersion in accordance with the present embodiment comprises a compound of Formula I or a pharmaceutically acceptable salt thereof, for example ABT-263 free base or ABT-263 bis-HCl, in an essentially non-crystalline or amorphous form, which is usually more soluble than the crystalline form. The term “solid dispersion” herein encompasses systems having small solid-state particles of one phase dispersed in another solid-state phase. More particularly, the present solid dispersions comprise one or more active ingredients dispersed in an inert carrier or matrix in solid state, and can be prepared by melting or solvent methods or by a combination of melting and solvent methods. According to the present embodiment a solvent method as described herein is particularly favored, avoiding the risk of thermal decomposition of the active ingredient by exposure to temperatures required to melt the polymeric carrier.

An “amorphous form” refers to a particle without definite structure, i.e., lacking crystalline structure.

The term “essentially non-crystalline” herein means that no more than about 5%, for example no more than about 2% or no more than about 1% crystallinity is observed by X-ray diffraction analysis. In a particular embodiment, no detectable crystallinity is observed by one or both of X-ray diffraction analysis or polarization microscopy.

ABT-263 bis-HCl, by virtue of its crystalline nature, is typically more convenient to use as an API than ABT-263 free base, which as prepared according to the '135 publication is an amorphous or glassy solid. However, there may be advantages in providing a solid dispersion formulation of ABT-263 wherein the ABT-263 is in free base form, as the drug will be less susceptible to crystallization within the formulation or immediately upon release therefrom. Thus in a particular embodiment, the composition comprises ABT-263 free base. It is emphasized that, in this embodiment, it is not necessarily the free base form of ABT-263 that is used as the API in preparing the composition.

The concentration of drug in the solid dispersion of the present embodiment is at least about 2.5%, e.g., about 2.5% to about 50%, by free base equivalent weight. Illustratively, for example where the drug is ABT-263, the drug concentration in various compositions is at least about 5%, e.g., about 5% to about 40%, for example about 5%, about 10%, about 15%, about 20%, about 25%, about 30%, about 35% or about 40%, by free base equivalent weight.

The major component of the matrix of a solid dispersion product is a polymer that is hydrophilic or water-soluble at least in a part of the pH scale, more particularly at a pH occurring in the gastrointestinal (GI) tract, or a combination of such polymers. A polymer or polymer mixture useful herein is solid at ambient temperature and, in the interests of good storage stability at a range of temperatures, should remain solid even at the highest temperatures typically experienced during storage, transport and handling of the product. A useful property of a polymer determining its usefulness herein is therefore its glass transition temperature (T_{g}). Suitable water-soluble polymers include, but are not limited to, those having a T_{g} of at least about 50° C., more particularly about 80° C. to about 180° C. Methods for determining T_{g} values of organic polymers are described for example in Sperling, ed. (1992) Introduction To Physical Polymer Science, 2nd edition, John Wiley & Sons, Inc.

Non-limiting examples of polymeric carriers useful herein include:
homopolymers and copolymers of N-vinyl lactams, especially homopolymers and copolymers of N-vinyl pyrrolidone, e.g., the homopolymer polyvinylpyrrolidone (PVP or povidone) and copolymers such as those comprising monomers of N-vinyl pyrrolidone and vinyl acetate (copovidone) or N-vinyl pyrrolidone and vinyl propionate;
cellulose esters and cellulose ethers, in particular methylcellulose, ethylcellulose, (hydroxyalkyl)celluloses such as hydroxypropylcellulose, (hydroxyalkyl)alkyl-celluloses such as hydroxypropylmethylcellulose (HPMC or hypromellose), cellulose phthalates and succinates such as cellulose acetate phthalate, hydroxypropylmethylcellulose phthalate, hydroxypropylmethylcellulose succinate and hydroxypropylmethylcellulose acetate succinate (HPMC-AS);
high molecular weight polyalkylene oxides such as polyethylene oxide, polypropylene oxide and copolymers of ethylene oxide and propylene oxide (poloxamers);
polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates) and poly(hydroxyalkyl methacrylates);
polyacrylamides;
vinyl acetate polymers such as copolymers of vinyl acetate and crotonic acid, partially hydrolyzed polyvinyl acetate (also referred to as partially saponified “polyvinyl alcohol”) and polyvinyl alcohol;
oligo- and polysaccharides such as carrageenans, galactomannans and xanthan gum;
   and mixtures of two or more thereof.

In some compositions, the solid dispersion matrix comprises one or more polymeric carriers selected from the group consisting of copovidone, povidone and HPMC-AS. A particular example of a useful copovidone is one consisting of about 60% N-vinyl pyrrolidone and about 40% vinyl acetate monomers. A particular example of a useful povidone is one having a K-value (a measure of viscosity of an aqueous solution of the povidone) of about 30.

One or more polymeric carriers typically constitute in total about 20% to about 90%, for example about 40% to about 85%, by weight of the solid dispersion.

Upon oral administration and exposure to GI fluid, it is believed without being bound by theory that, through interplay between the polymeric carrier and a surfactant component of the solid dispersion, a suitable release rate and inhibition of crystallization or recrystallization of the active ingredient are provided, thereby permitting bioabsorption.

Particularly useful as surfactants in solid dispersions of the present embodiment are pharmaceutically acceptable non-ionic surfactants, especially those having a hydrophilic-lipophilic balance (HLB) value of about 12 to about 18, for example about 13 to about 17, or about 14 to about 16. The HLB system (see Fiedler (2002) Encyclopedia of Excipients, 5th edition, Aulendorf: ECV-Editio-Cantor-Verlag) attributes numeric values to surfactants, with lipophilic substances receiving lower HLB values and hydrophilic substances receiving higher HLB values.

Non-limiting examples of non-ionic surfactants useful in compositions of the present embodiment include:
polyoxyethylene castor oil derivatives such as PEG-35 castor oil (e.g., Cremophor EL™ of BASF Corp. or equivalent product), PEG-40 hydrogenated castor oil (e.g., Cremophor RH 40™ or equivalent product) and PEG-60 hydrogenated castor oil (e.g., Cremophor RH™ 60 or equivalent product);
fatty acid monoesters of sorbitan, for example sorbitan monooleate (e.g., Span™ 80 or equivalent product), sorbitan monostearate (e.g., Span™ 60 or equivalent product), sorbitan monopalmitate (e.g., Span™ 40 or equivalent product) and sorbitan monolaurate (e.g., Span™ 20 or equivalent product);
fatty acid monoesters of polyoxyethylene sorbitan (polysorbates) such as PEG-20 sorbitan monooleate (polysorbate 80, e.g., Tween™ 80 or equivalent product) PEG-20 sorbitan monostearate (polysorbate 60, e.g., Tween™ 60 or equivalent product), PEG-20 sorbitan monopalmitate (polysorbate 40, e.g., Tween™ 40 or equivalent product), or PEG-20 sorbitan monolaurate (polysorbate 20, e.g., Tween™ 20 or equivalent product);
poloxamers such as poloxamer 124, poloxamer 188, poloxamer 237, poloxamer 388 or poloxamer 407;
α-tocopheryl polyethylene glycol succinate (TPGS or vitamin E polyethylene glycol succinate, see U.S. National Formulary);
   and mixtures of two or more thereof.

One or more surfactants typically constitute in total about 2% to about 25%, for example about 5% to about 20%, by weight of the solid dispersion.

A dosage form of the present embodiment can consist of, or consist essentially of, a solid dispersion as described above. However, in some cases a dosage form of the present embodiment contains additional excipients and requires additional processing of the solid dispersion. For example, the solid dispersion can be ground to a powder and filled into a capsule shell or molded or compressed to form a tablet, with additional excipients as may be conventionally used in such dosage forms.

Thus orally deliverable solid dosage forms of the present embodiment include but are not limited to capsules, dragees, granules, pills, powders and tablets. Excipients commonly used to formulate such dosage forms include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, preservatives, propellants, releasing agents, sterilizing agents, sweeteners, solubilizers and mixtures thereof. Examples of specific excipients include agar, alginic acid, aluminum hydroxide, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, talc, tragacanth, water, safflower oil, sesame oil, sodium carboxymethyl cellulose, sodium lauryl sulfate, sodium phosphate salts, soybean oil, sucrose, tetrahydrofurfuryl alcohol, and mixtures thereof.

A solvent process for preparing a solid dispersion as described above comprises dissolving the API, the polymeric carrier and the surfactant in a suitable solvent; and removing the solvent to provide the solid dispersion. Optionally, where the API is in salt form and it is desired to provide a solid dispersion of the drug in free base form, a base is added before solvent removal to effect conversion of the API to its corresponding free base. For example, where the API is ABT-263 bis-HCl, addition of a base such as sodium hydroxide (NaOH), potassium hydroxide (KOH), sodium bicarbonate (NaHCO₃), potassium bicarbonate (KHCO₃) or ammonium bicarbonate (NH₄HCO₃) in an amount of at least 2 moles per mole of API can result in conversion of the API to ABT-263 free base. The inorganic salt by-product, illustratively NaCl, KCl or NH₄Cl, can remain in the product or is optionally extracted before solvent removal.

In the dissolving step, the various components can be added in any order. For example, each ingredient can be added to the solvent separately and then dissolved therein. Alternatively, the polymeric carrier and/or surfactant can be pre-mixed with the API, and the resulting mixture then added to the solvent. However, it will generally be found convenient, when the process includes in situ salt-to-free base conversion, to first add the API salt and the base to the solvent, then (optionally after extraction of a salt by-product) add the polymeric carrier and surfactant.

In principle any solvent can be used so long as it is effective to dissolve the active ingredient, polymer carrier and surfactant. Non-limiting examples of solvents that can be useful include methanol, ethanol, acetone and mixtures thereof. Optionally a cosolvent can be included.

Where it is desired to extract a salt by-product such as NaCl, KCl or NH₄Cl prior to solvent removal, a solvent can be selected wherein the salt by-product is insoluble, thereby permitting extraction of the salt by-product by filtration.

Solvent removal can be accomplished using heat, vacuum or a combination thereof. If heat is used, it is generally preferable to avoid exceeding the glass transition temperature (T_{g}) of the polymeric matrix. For most purposes heating at a temperature of about 50° C. to about 80° C., for example about 55° C. to about 75° C., will be found suitable. After solvent removal, the resulting product is cooled (if necessary) to ambient temperature.

Further process details can be found in the illustrative processes of Examples 16 and 17 below.

### Fifth Composition Embodiment

A composition of the fifth embodiment set forth hereinabove comprises an orally deliverable pharmaceutical dosage form comprising a solid dispersion or solid solution that comprises (a) a compound of Formula I or a pharmaceutically acceptable salt thereof in a free base equivalent amount of at least about 2.5% by weight of the composition, (b) at least one pharmaceutically acceptable polymer and (c) at least one pharmaceutically acceptable solubilizer.

In dosage forms of the present embodiment, the active ingredient is present as a solid dispersion or as a solid solution. The term “solid dispersion” in relation to the present embodiment defines a system in a solid state (as opposed to a liquid or gaseous state) comprising at least two components, wherein one component is dispersed evenly throughout the other component or components. For example, the active ingredient or combination of active ingredients is dispersed in a matrix comprising the pharmaceutically acceptable polymer(s) and pharmaceutically acceptable solubilizers. The term “solid dispersion” encompasses systems having small particles, typically less than 1 μm in diameter, of one phase dispersed in another phase. When said dispersion is such that the system is chemically and physically uniform or homogeneous throughout or consists of one phase (as defined in thermodynamics), such a solid dispersion will be called a “solid solution” or a “glassy solution”. A glassy solution is a homogeneous, glassy system in which a solute is dissolved in a glassy solvent. Glassy solutions and solid solutions are preferred physical systems according to the present embodiment. These systems do not contain any significant amount of active ingredients in a crystalline or microcrystalline state, as evidenced by thermal analysis (DSC) or X-ray diffraction analysis (WAXS).

Dosage forms according to the present embodiment are characterized by excellent stability and, in particular, exhibit high resistance against recrystallization or decomposition of the active ingredient(s).

Dosage forms of the present embodiment exhibit a release and absorption behavior that is characterized by relatively high attainable AUC, relatively high attainable Cₘₐₓ, and relatively low Tₘₐₓ.

A dispersion formed upon contact of a dosage form of the present embodiment with an aqueous liquid may also be useful as such, for example as an oral liquid dosage form or a parenteral injection.

Generally, the solid dispersion product of the present embodiment comprises
(a) about 2.5% to about 40%, preferably about 2.5% to about 25%, by weight of a compound of Formula I or a salt thereof, for example ABT-263 free base, ABT-263 bis-HCl or ABT-263 sodium salt,
(b) about 40% to about 95%, preferably about 50% to about 94%, by weight of at least one pharmaceutically acceptable polymer,
(c) about 2% to about 20%, preferably about 5% to about 20%, by weight of at least one solubilizer, and
(d) zero to about 15%, preferably zero to 10%, by weight of additives.

Whereas the dosage form of the present embodiment may consist entirely of solid dispersion product, additives and adjuvants can be used in formulating the solid dispersion product into the dosage form. Generally, the dosage form comprises at least about 10%, preferably at least about 40%, and most preferably at least about 45%, by weight of solid dispersion product, based on the total weight of the solid dosage form.

Typically, a single dosage form of the present embodiment contains about 50 mg to about 1000 mg, preferably about 75 mg to about 600 mg, in particular about 100 mg to about 500 mg, of free base equivalent of a compound of Formula I, for example ABT-263, or a salt thereof.

In suitable embodiments, the active ingredient is selected from the group consisting of the free base, the sodium salt and the bis-hydrochloride salt of ABT-263, and combinations thereof. In a preferred embodiment the active ingredient is ABT-263 free base.

The term “solubilizer” as used in relation to the present embodiment refers to a pharmaceutically acceptable nonionic or anionic surfactant. The solubilizer may effect an instantaneous emulsification of the active ingredient released from the dosage form and/or prevent precipitation of the active ingredient in the aqueous fluid of the gastrointestinal tract. A single solubilizer or combination of solubilizers may be used. The solubilizer may be selected from the group consisting of nonionic solubilizers, anionic solubilizers and combinations thereof. In some compositions of the present embodiment, the solid dispersion product comprises a combination of two or more pharmaceutically acceptable solubilizers.

Illustratively, a nonionic solubilizer can be selected from the group consisting of polyol fatty acid esters, polyalkoxylated polyol fatty acid esters, polyalkoxylated fatty alcohol ethers, tocopheryl compounds or mixtures of two or more thereof, and an anionic solubilizer can be selected from the group consisting of alkyl sulfates, alkylcarboxylates, alkylbenzole sulfates and secondary alkane sulfonates.

Preferred nonionic solubilizers are selected from sorbitan fatty acid esters, polyalkoxylated fatty acid esters such as, for example, polyalkoxylated glycerides, polyalkoxylated sorbitan fatty acid esters and fatty acid esters of polyalkylene glycols, polyalkoxylated ethers of fatty alcohols, tocopheryl compounds, and mixtures of two or more thereof. A fatty acid chain in these solubilizer compounds ordinarily comprises 8 to 22 carbon atoms. Polyalkylene oxide blocks comprise on average 4 to 50 alkylene oxide units, preferably ethylene oxide units, per molecule.

Examples of suitable sorbitan fatty acid esters are sorbitan monolaurate, sorbitan monopalmitate, sorbitan monostearate (e.g., Span™ 60), sorbitan monooleate (e.g., Span™ 80), sorbitan tristearate, sorbitan trioleate or sorbitan monolaurate.

Examples of suitable polyalkoxylated sorbitan fatty acid esters are polyoxyethylene (20) sorbitan monolaurate, polyoxyethylene (20) sorbitan monopalmitate, polyoxyethylene (20) sorbitan monostearate, polyoxyethylene (20) sorbitan monooleate (e.g., Tween™ 80), polyoxyethylene (20) sorbitan tristearate (e.g., Tween™ 65), polyoxyethylene (20) sorbitan trioleate (e.g., Tween™ 85), polyoxyethylene (4) sorbitan monostearate, polyoxyethylene (4) sorbitan monolaurate or polyoxyethylene (4) sorbitan monooleate.

Suitable polyalkoxylated glycerides are obtained for example by alkoxylation of natural or hydrogenated glycerides or by transesterification of natural or hydrogenated glycerides with polyalkylene glycols. Commercially available examples are polyoxyethylene glycerol ricinoleate 35, polyoxyethylene glycerol trihydroxystearate 40 (e.g., Cremophor RH™ 40 of BASF AG) and polyalkoxylated glycerides including those obtainable under the proprietary names Gelucire™ and Labrafil™ from Gattefossé, e.g., Gelucire™ 44/14 (lauroyl macrogol 32 glycerides prepared by transesterification of hydrogenated palm kernel oil with PEG-1500), Gelucire™ 50/13 (stearoyl macrogol 32 glycerides, prepared by transesterification of hydrogenated palm oil with PEG-1500) or Labrafil™ M 1944 CS (oleoyl macrogol 6 glycerides prepared by transesterification of apricot kernel oil with PEG-300).

A suitable fatty acid ester of polyalkylene glycols is, for example, PEG-660 hydroxystearic acid (polyglycol ester of 12-hydroxystearic acid (70 mol %) with 30 mol % ethylene glycol).

Suitable polyalkoxylated ethers of fatty alcohols are, for example, PEG (2) stearyl ether (e.g., Brij™ 72), macrogol 6 cetylstearyl ether or macrogol 25 cetylstearyl ether.

In general, a tocopheryl compound useful herein corresponds to the formula

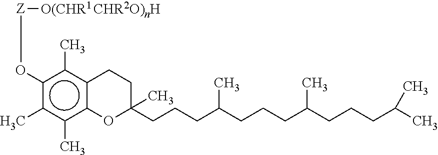

wherein Z is a linking group, R¹ and R² are, independently of one another, hydrogen or C₁-C₄ alkyl and n is an integer from 5 to 100, preferably 10 to 50. Typically, Z is the residue of an aliphatic dibasic acid such as glutaric, succinic or adipic acid. Preferably, both R¹ and R² are hydrogen.

A preferred tocopheryl compound is α-tocopheryl polyethylene glycol succinate, available for example as the proprietary product Vitamin E TPGS™. This is a water-soluble derivative of natural-source vitamin E prepared by esterifying D-α-tocopheryl acid succinate with PEG-1000.

According to one preferred embodiment the pharmaceutically acceptable solubilizer is selected from the group consisting of tocopheryl compounds having a polyalkylene glycol moiety (such as α-tocopheryl polyethylene glycol succinate), sorbitan fatty acid esters (such as sorbitan monolaurate) and polyoxyethylene sorbitan fatty acid esters (such as polyoxyethylene sorbitan monolaurate) and combinations of two or more thereof. This embodiment is particularly useful where the active ingredient is ABT-263 free base.

In another preferred embodiment the dosage form comprises at least one pharmaceutically acceptable nonionic solubilizer and at least one pharmaceutically acceptable anionic solubilizer. Preferably, the nonionic solubilizer is selected from the group consisting of sorbitan fatty acid esters, polyoxyethylene sorbitan fatty acid esters and α-tocopheryl polyethylene glycol succinate; and the anionic solubilizer is sodium lauryl sulfate (also referred to herein as SDS). This embodiment is particularly useful where the active ingredient is an acid addition salt of ABT-263 such as ABT-263 bis-HCl.

Formation of a solid solution can be promoted by incorporating a non-volatile solvent for the active ingredient into the solid dispersion product. The non-volatile solvent is suitably selected from solvents with high dissolving power for a compound of Formula I, for example ABT-263, which are liquid at ambient temperature and ambient pressure.

Nonlimiting examples of suitable solvents include liquid polyethylene glycols, e.g., PEG-400; N-methylpyrrolidone; 1,3-bis(pyrrolidon-1-yl)butane; and propylene glycol. A preferred solvent is propylene glycol. The amount of the non-volatile solvent to be used should not be so high as to compromise the mechanical properties of the solid dispersion product and usually is about 2% to about 10%, for example about 3% to about 5%, by weight of the solid dispersion product.

The pharmaceutically acceptable polymer may be selected from water-soluble polymers, water-dispersible polymers, water-swellable polymers and mixtures thereof. Polymers are considered water-soluble if they form a clear homogeneous solution in water. When dissolved at 20° C. in an aqueous solution at 2% (w/v), the water-soluble polymer preferably has an apparent viscosity of about 1 to about 5,000 mPa·s, more preferably about 1 to about 700 mPa·s, and most preferably about 5 to about 100 mPa·s. Water-dispersible polymers are those that, when contacted with water, form colloidal dispersions rather than a clear solution. Upon contact with water or aqueous solutions, water-swellable polymers typically form a rubbery gel.

Preferably, the pharmaceutically acceptable polymer employed in compositions of the present embodiment has a T_{g} of at least about 40° C., preferably at least about 50° C., most preferably about 80° C. to about 180° C. The T_{g} value of a copolymer can be calculated as the weighted sum of the T_{g} values for homopolymers derived from each of the individual monomers, i, that make up the copolymer: T_{g}=ΣWᵢXᵢ where Wᵢ is the weight percent of monomer i in the copolymer, and X, is the T_{g} value for the homopolymer derived from monomer i. T_{g} values for homopolymers may be taken from Brandrup & Immergut, eds. (1975) Polymer Handbook, 2nd edition, John Wiley & Sons, Inc.

Various additives contained in the solid dispersion product or even the active ingredient itself may exert a plasticizing effect on the polymer and thus depress the T_{g} of the polymer such that the final solid dispersion product has a somewhat lower T_{g} than the starting polymer used for its preparation. In general, the final solid dispersion product has a T_{g} of 20° C. or higher, preferably 25° C. or higher, more preferably 30° C. or higher and most preferably 40° C. or higher, e.g., a T_{g} from about 45° C. to about 60° C.

For example, preferred pharmaceutically acceptable polymers can be selected from the group comprising homopolymers and copolymers of N-vinyl lactams, especially homopolymers and copolymers of N-vinyl pyrrolidone, e.g., polyvinylpyrrolidone (PVP), copolymers of N-vinyl pyrrolidone and vinyl acetate or vinyl propionate, cellulose esters and cellulose ethers, in particular methylcellulose and ethylcellulose, hydroxyalkylcelluloses, in particular hydroxypropylcellulose, hydroxyalkylalkylcelluloses, in particular hydroxypropyl-methylcellulose, cellulose phthalates and succinates, in particular cellulose acetate phthalate and hydroxypropylmethylcellulose phthalate, hydroxypropylmethylcellulose succinate and hydroxypropylmethylcellulose acetate succinate; high molecular polyalkylene oxides such as polyethylene oxide and polypropylene oxide and copolymers of ethylene oxide and propylene oxide; polyvinyl alcohol/polyethylene glycol graft copolymers (available as Kollicoat™ IR from BASF AG); polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates) and poly(hydroxyalkyl methacrylates); polyacrylamides; vinyl acetate polymers such as copolymers of vinyl acetate and crotonic acid; partially hydrolyzed polyvinyl acetate (also referred to as partially saponified “polyvinyl alcohol”); polyvinyl alcohol; oligo- and polysaccharides such as carrageenans, galactomannans and xanthan gum, and mixtures of two or more thereof.

Among these, homopolymers or copolymers of N-vinyl pyrrolidone, in particular a copolymer of N-vinyl pyrrolidone and vinyl acetate, are preferred. A particularly preferred polymer is a copolymer of 60% by weight N-vinyl pyrrolidone and 40% by weight vinyl acetate.

A further polymer which can be suitably used is a mixture of PVP and polyvinylacetate as sold, for example, under the proprietary name Kollidon® SR of BASF AG.

A solid dispersion product of the present embodiment may be prepared by a variety of methods.

Preferably, the solid dispersion product is prepared by melt-extrusion. Accordingly, the solid dispersion product is a melt-processed, solidified mixture. The melt-extrusion process comprises preparing a homogeneous melt of an active ingredient or combination of active ingredients, the pharmaceutically acceptable polymer and the solubilizer, and cooling the melt until it solidifies.

“Melting” in the present context means a transition into a liquid or rubbery state in which it is possible for one component to become homogeneously embedded in the other. Typically, one component will melt and the other components will dissolve in the melt, thus forming a solution. Melting usually involves heating above the softening point of the pharmaceutically acceptable polymer. Preparation of the melt can take place in a variety of ways. Mixing of the components can take place before, during or after formation of the melt. For example, the components can be mixed first and then melted, or they can be simultaneously mixed and melted. Usually, the melt is homogenized in order to disperse the active ingredient efficiently. Also, it may be convenient first to melt the pharmaceutically acceptable polymer and then to admix and homogenize the active ingredient.

Usually, the melt temperature is in the range of about 70° C. to about 250° C., preferably about 80° C. to about 180° C., and most preferably about 100° C. to about 140° C.

The active ingredient can be employed as such or as a solution or dispersion in a suitable solvent such as one or more alcohols, aliphatic hydrocarbons or esters. Another solvent which can be used is liquid carbon dioxide. The solvent is removed, e.g., evaporated, upon preparation of the melt. Alternatively, solid dispersions of the active ingredient can be prepared with a non-volatile solvent for the active ingredient as previously mentioned.

Various additives may be included in the melt, for example flow regulators such as colloidal silica, lubricants, bulking agents (fillers), disintegrants, plasticizers, stabilizers such as antioxidants, light stabilizers, radical scavengers, or stabilizers against microbial attack.

The melting and/or mixing takes place in an apparatus customary for such a purpose. Particularly suitable are extruders or kneaders. Suitable extruders include single screw extruders, intermeshing screw extruders or multiscrew extruders, preferably twin-screw extruders, which can be corotating or counterrotating and, optionally, equipped with kneading disks or other screw elements for mixing or dispersing the melt. It will be appreciated that the working temperatures will be determined by the kind of extruder or the kind of configuration within the extruder used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The extrudate exiting from the extruder ranges from pasty to viscous. Before allowing the extrudate to solidify, the extrudate may be directly shaped into virtually any desired shape. Shaping of the extrudate may be conveniently carried out by a calendar with two counter-rotating rollers with mutually matching depressions on their surface. A broad range of tablet forms can be attained by using rollers with different forms of depressions. If the rollers do not have depressions on their surface, films can be obtained. Alternatively, the extrudate is moulded into the desired shape by injection-moulding. Alternatively, the extrudate is subjected to profile extrusion and cut into pieces, either before (hot-cut) or after solidification (cold-cut).

Additionally, foams can be formed if the extrudate contains a propellant such as a gas, e.g., carbon dioxide, or a volatile compound, e.g., a low molecular-weight hydrocarbon, or a compound that is thermally decomposable to a gas. The propellant is dissolved in the extrudate under the relatively high pressure conditions within the extruder and, when the extrudate emerges from the extruder die, the pressure is suddenly released. Thus the solvability of the propellant is decreased and/or the propellant vaporizes so that a foam is formed.

Optionally, the resulting solid solution product is milled or ground to granules. The granules may then be filled into capsules or may be compacted. Compacting means a process whereby a powder mass comprising the granules is densified under high pressure in order to obtain a compact with low porosity, e.g., a tablet. Compression of the powder mass is usually done in a tablet press, more specifically in a steel die between two moving punches.

Preferably, the solid dosage form contains at least one additive selected from flow regulators, disintegrants, bulking agents and lubricants.

At least one additive selected from flow regulators, disintegrants, bulking agents (fillers) and lubricants is preferably used in compacting the granules. Disintegrants promote a rapid disintegration of the compact in the stomach and help the liberated granules separate from one another. Suitable disintegrants are crosslinked polymers such as crosslinked PVP (crospovidone) and crosslinked sodium carboxymethylcellulose. Suitable bulking agents (also referred to as “fillers”) can be selected from mannitol, lactose, calcium hydrogen phosphate, microcrystalline cellulose (e.g., Avicel™), magnesium oxide, potato and corn starches, isomalt and polyvinyl alcohol. Suitable flow regulators can be selected from highly dispersed silica (e.g., Aerosil™) (also referred to as colloidal silicon dioxide), and animal and vegetable fats and waxes. A lubricant is preferably used in compacting the granules. Suitable lubricants can be selected from polyethylene glycol (e.g., having a molecular weight of about 1,000 to about 6,000), magnesium and calcium stearates, sodium stearyl fumarate, talc, and the like.

Various other additives may be used, for example dyes such as azo dyes, organic or inorganic pigments such as aluminum oxide or titanium dioxide, or dyes of natural origin; stabilizers such as antioxidants, light stabilizers, radical scavengers, or stabilizers against microbial attack. Such additives are known to those skilled in the art, and non-limiting examples include Vitamin E and derivatives thereof (e.g., Vitamin E-TPGST™), butylhydroxytoluene (BTH), cysteine, and ascorbic acid and derivatives thereof.

Dosage forms according to the present embodiment may consist of several layers, as for example in laminated or multilayer tablets. They can be in open or closed form. “Closed dosage forms” are those in which one layer is completely surrounded by at least one other layer. Multilayer forms have the advantage that two active ingredients which are incompatible with one another can be processed, or that the release characteristics of the active ingredient(s) can be controlled. For example, it is possible to provide an initial dose by including an active ingredient in an outer layer, and a maintenance dose by including the active ingredient in an inner layer. Multilayer tablet types may be produced by compressing two or more layers of granules. Alternatively, multilayer dosage forms may be produced by a process known as “coextrusion”. In essence, the process comprises preparation of at least two different melt compositions as explained above, and passing these molten compositions into a joint coextrusion die. The shape of the coextrusion die depends on the required drug form. For example, dies with a plain die gap, called slot dies, and dies with an annular slit are suitable.

In order to facilitate oral administration of such a dosage form, it is advantageous to give the dosage form an appropriate shape. Large tablets are therefore preferably elongated rather than round in shape, to facilitate comfortable swallowing.

An optional film coat on the tablet further contributes to ease of swallowing. A film coat also improves taste and provides an elegant appearance. If desired, the film coat may be an enteric coat. The film coat usually includes a polymeric film-forming material such as hydroxypropylmethylcellulose, hydroxypropylcellulose, or an acrylate or methacrylate copolymer. Besides a film-forming polymer, the film coat may further comprise a plasticizer, e.g., polyethylene glycol, a surfactant, e.g., a polyoxyethylene sorbitan ester, and optionally a pigment, e.g., titanium dioxide or iron oxide. The film coat may also comprise talc as an anti-adherent. The film coat if present usually accounts for less than about 5% by weight of the dosage form.

In an alternative process for preparing a solid dosage form, the solid dispersion product is ground and filled into a capsule shell. Suitable materials for capsule shells are known in the art, and include for example gelatin, gums such as carrageenan or gellan, and cellulose or cellulose derivatives such as hydroxypropylmethylcellulose.

It has been found that a solid dispersion of ABT-263 according to the present embodiment not only shows adequate bioavailability after oral administration but also results in a storage-stable, ready-to-use dosage form. Quite surprisingly, in such a solid dispersion the ABT-263 molecule, despite its essentially non-crystalline amorphous state, is largely resistant against oxidation even in presence of only a minor amount of antioxidant or absence of any antioxidant.

However, optionally an HCA, for example a sulfur-containing antioxidant, can be included in the composition of the present embodiment if so desired.

### Sixth Composition Embodiment

A composition of the sixth embodiment set forth hereinabove comprises (a) a compound of Formula I or a pharmaceutically acceptable salt thereof, for example ABT-263 free base or ABT-263 bis-HCl, in solid particulate form and in a free base equivalent amount of at least about 2.5% by weight of the composition, and (b) a plurality of pharmaceutically acceptable excipients including at least a solid diluent and a solid disintegrant.

Illustratively, the active ingredient concentration in a composition of the present embodiment is at least about 5%, at least about 10%, at least about 15%, at least about 20%, at least about 25% or at least about 30% by weight of the formulation, and can be as high as 40% by weight or, in some instances, even higher.

It is generally preferred that the solid particulate form of the active ingredient used in the composition should be a crystalline form. In the case of ABT-263, the product prepared by the process described in the '135 publication is non-crystalline and is generally unsuitable for formulation as a solid dosage form of the present embodiment. For this reason, the composition preferably contains as API a crystalline form of the free base, e.g., ABT-263 free base crystalline Form I or Form II as described hereinabove, or a crystalline salt, such as ABT-263 bis-HCl.

Particle size of the API is not narrowly critical, though results suggest that reduction in particle size can improve bioavailability. In compositions of the invention, the D₉₀ particle size (90% by volume of the API particles in their longest dimension are smaller than this) is typically about 2.5 to about 50 μm, for example about 3 to about 30 μm. API in the upper part of this D₉₀ range is typically unmilled. Reduction in particle size to the lower part of the D₉₀ range is achievable, for example, by pin-milling or jet-milling. In some compositions, unmilled API having a D₉₀ of about 20 to about 30 μm is used. In other compositions, pin-milled or jet-milled API having a D₉₀ of about 3 to about 10 μm is used. In still other compositions, API of intermediate D₉₀, for example about 10 to about 20 μm, is used.

A composition of the present embodiment comprises, in addition to the API, a plurality of pharmaceutically acceptable excipients including at least one or more solid diluents and one or more solid disintegrants. Optionally, the excipients further include one or more binding agents, wetting agents and/or antifrictional agents (lubricants, anti-adherents and/or glidants). Many excipients have two or more functions in a pharmaceutical composition. Characterization herein of a particular excipient as having a certain function, e.g., diluent, disintegrant, binding agent, etc., should not be read as limiting to that function. Further information on excipients can be found in standard reference works such as Kibbe, ed. (2000) Handbook of Pharmaceutical Excipients, 3rd edition, Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 95%, for example about 20% to about 90%, or about 50% to about 85%, by weight of the composition. The diluent or diluents selected preferably exhibit suitable flow properties and, where tablets are desired, compressibility.

Microcrystalline cellulose and silicified microcrystalline cellulose are particularly useful diluents, and are optionally used in combination with a water-soluble diluent such as mannitol. Illustratively, a suitable weight ratio of microcrystalline cellulose or silicified microcrystalline cellulose to mannitol is about 10:1 to about 1:1, but ratios outside this range can be useful in particular circumstances.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.5% to about 20%, or about 1% to about 10%, by weight of the composition.

Sodium starch glycolate is a particularly useful disintegrant, and typically constitutes in total about 1% to about 20%, for example about 2% to about 15%, or about 5% to about 10%, by weight of the composition.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone or PVP), for example povidone K-15, K-30 and K-29/32; polyacrylic acids (carbomers); polymethacrylates; and the like. One or more binding agents and/or adhesives, if present, typically constitute in total about 0.5% to about 25%, for example about 1% to about 15%, or about 1.5% to about 10%, by weight of the composition.

Povidone and hydroxypropylcellulose, either individually or in combination, are particularly useful binding agents for tablet formulations, and, if present, typically constitute about 0.5% to about 15%, for example about 1% to about 10%, or about 2% to about 8%, by weight of the composition.

Wetting agents, if present, are normally selected to maintain the drug in close association with water, a condition that can improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters, for example sorbitan monolaurate, sorbitan monooleate, sorbitan monopalmitate and sorbitan monostearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; α-tocopherol polyethylene glycol (1000) succinate (TPGS); tyloxapol; and the like. One or more wetting agents, if present, typically constitute in total about 0.1% to about 15%, for example about 0.2% to about 10%, or about 0.5% to about 7%, by weight of the composition.

Nonionic surfactants, more particularly poloxamers, are examples of wetting agents that can be useful herein. Illustratively, a poloxamer such as Pluronic™ F127, if present, can constitute about 0.1% to about 10%, for example about 0.2% to about 7%, or about 0.5% to about 5%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 5%, or about 0.2% to about 2%, by weight of the composition. Sodium stearyl fumarate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.05% to about 10%, for example about 0.1% to about 7%, or about 0.2% to about 5%, by weight of the composition. Colloidal silicon dioxide is a particularly useful anti-adherent.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.05% to about 10%, for example about 0.1% to about 7%, or about 0.2% to about 5%, by weight of the composition. Colloidal silicon dioxide is a particularly useful glidant.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin (in the form of hard gelatin capsules or soft elastic gelatin capsules), starch, carrageenan and/or HPMC, optionally together with one or more plasticizers.

Solid dosage forms according to the present embodiment not only show adequate bioavailability after oral administration but exhibit acceptable storage-stability, being relatively resistant to oxidative degradation of the active ingredient even in presence of only a minor amount of antioxidant or absence of any antioxidant.

However, optionally an HCA, for example a sulfur-containing antioxidant, can be included in the composition of the present embodiment if so desired.

Any suitable process of pharmacy can be used to prepare a composition of the present embodiment, including dry blending with or without direct compression, and wet or dry granulation. In the illustrative, non-limiting processes and compositions shown below, API can be used in unmilled form, e.g., with a D₉₀ particle size of about 20 to about 30 μm, or after milling to a desired size, e.g., pin-milled or jet-milled to a D₉₀ particle size of about 3 to about 10 μm.

An illustrative dry blending process is as follows. API (e.g., ABT-263 bis-HCl) is mixed with excipients except lubricant, for example by blending in a V-blender for approximately 20 minutes. Lubricant is then added. The resulting powder blend is compressed, for example at 500 lb, in a tablet press with suitable tooling to provide the size and shape of tablets desired. Alternatively, the powder blend is filled into capsules.

An illustrative composition prepared by the above process consists of the following ingredients (all percentages by weight):

| | |
|---|---|
| ABT-263 bis-HCl | 10.75% |
| | (10% free base equivalent) |
| silicified microcrystalline cellulose | 49.00% |
| mannitol | 20.00% |
| pregelatinized starch | 5.00% |
| sodium starch glycolate | 10.00% |
| poloxamer (Pluronic ™ F127) | 4.00% |
| colloidal silicon dioxide | 1.00% |
| sodium stearyl fumarate | 0.25% |

Tablets of 50 mg ABT-263 dosage strength (total tablet weight 500 mg) are prepared from the above ingredients in a Carver press at 500 lb, with round tooling.

A first illustrative wet granulation process is as follows. API (e.g., ABT-263 bis-HCl) is suspended in a binder/surfactant solution (granulation liquid), then added to a blend of diluent(s) and disintegrant(s) in a food processor to prepare a granulate.

A second illustrative wet granulation process is as follows. API (e.g., ABT-263 bis-HCl) is mixed with excipients, including granulation liquid but excluding lubricant, and granulated in a food processor. The granules are dried and passed through a 20 mesh screen. Lubricant is then added.

A third illustrative wet granulation process is as follows. API (e.g., ABT-263 bis-HCl) is mixed with excipients, including granulation liquid and a first amount of disintegrant (intragranular excipients) but excluding lubricant, and granulated in a food processor. The granules are dried and passed through a 20 mesh screen. A second amount of disintegrant, lubricant and optionally other extragranular excipient(s) are then added.

Granules prepared by any of the above wet granulation processes can be compressed, for example at 500 lb, in a tablet press with suitable tooling to provide the size and shape of tablets desired. Alternatively, the granules can be filled into capsules.

A first illustrative tablet composition that can be prepared by any of the above wet granulation processes consists of the following ingredients (all percentages by weight):

| | |
|---|---|
| ABT-263 bis-HCl | 10.75% |
| | (10% free base equivalent) |
| microcrystalline cellulose | 83.50% |
| povidone K-30 | 3.00% |
| crospovidone | 1.50% |
| poloxamer (Pluronic ™ F127) | 1.00% |
| sodium stearyl fumarate | 0.25% |

A second illustrative tablet composition that can be prepared by any of the above wet granulation processes consists of the following ingredients (all percentages by weight):

| | |
|---|---|
| ABT-263 bis-HCl | 5.38% |
| | (5% free base equivalent) |
| microcrystalline cellulose | 85.87% |
| povidone K-30 | 3.00% |
| crospovidone | 1.50% |
| poloxamer (Pluronic ™ F127) | 4.00% |
| sodium stearyl fumarate | 0.25% |

A third illustrative tablet composition that can be prepared by any of the above wet granulation processes consists of the following ingredients (all percentages by weight):

| | |
|---|---|
| ABT-263 bis-HCl | 10.75% |
| | (10% free base equivalent) |
| microcrystalline cellulose | 50.00% |
| mannitol | 20.00% |
| povidone K-30 | 5.00% |
| sodium starch glycolate | 10.00% |
| poloxamer (Pluronic ™ F127) | 4.00% |
| sodium stearyl fumarate | 0.25% |

Tablets containing a 50 mg dose of ABT-263 are prepared from any of the above wet granulations.

An illustrative capsule composition that can be prepared by any of the above wet granulation processes consists of the following ingredients (all percentages by weight):

| | |
|---|---|
| ABT-263 bis-HCl | 10.75% |
| | (10% free base equivalent) |
| microcrystalline cellulose | 50.00% |
| mannitol | 30.00% |
| hydroxypropylcellulose | 3.00% |
| sodium starch glycolate | 5.00% |
| poloxamer (Pluronic ™ F127) | 1.00% |
| sodium stearyl fumarate | 0.25% |

The composition is filled into size 0 capsules.

### Bioavailability and Administration

In any of the above embodiments, and others not fully described herein but evident to the ordinarily skilled reader of the present specification, the formulation ingredients and amounts thereof can be selected to provide enhanced bioabsorption by comparison with a standard solution of the drug when administered orally. Such enhanced bioabsorption versus the standard solution can be evidenced, for example, by a pharmacokinetic (PK) profile having one or more of a higher Cₘₐₓ or an increased bioavailability as measured by AUC, for example AUC₀₋₂₄ or AUC_{0-∞}. Illustratively, bioavailability can be expressed as a percentage, for example using the parameter F, which computes AUC for oral delivery of a test composition as a percentage of AUC for intravenous (i.v.) delivery of the drug in a suitable solvent, taking into account any difference between oral and i.v. doses.

The standard solution in the case of ABT-263 can be, for example, a solution of ABT-263 free base in a carrier consisting of 10% DMSO in PEG-400, or a formulation referenced herein as “Formulation C”, which is a solution of ABT-263 bis-HCl solution at a free base equivalent concentration of 25 mg/ml in a carrier liquid consisting of 90% phosphatidylcholine+medium chain triglycerides 53/29 and 10% dehydrated alcohol USP (meeting standards set forth in the United States Pharmacopeia).

Bioavailability can be determined by PK studies in humans or in any suitable model species. For present purposes, a dog model is generally suitable. In various illustrative embodiments, where the drug is ABT-263 or a salt thereof, compositions of the invention exhibit oral bioavailability of at least about 15%, at least about 30%, at least about 35% or at least about 40%, up to or exceeding about 50%, in a dog model, when administered as a single dose of about 2.5 to about 10 mg/kg to fasting or non-fasting animals.

In one example, the composition comprises ABT-263 or a salt thereof and a carrier comprising ingredients and amounts thereof selected to provide a PK profile upon oral administration of the composition in a non-fasting dog model exhibiting a bioavailability of at least about 15%.

In one example, the composition comprises ABT-263 or a salt thereof and a carrier comprising ingredients and amounts thereof selected to provide a PK profile upon oral administration of the composition in a non-fasting dog model exhibiting a bioavailability of at least about 30%.

In one example, the composition comprises ABT-263 or a salt thereof and a carrier comprising ingredients and amounts thereof selected to provide a PK profile upon oral administration of the composition in a non-fasting dog model exhibiting a bioavailability of at least about 40%.

The potential of the present invention to provide bioavailability, for example of ABT-263, substantially greater, for example at least about 1.5× or at least about 2× greater, than that of the solution in 10% DMSO in PEG-400 described in above-cited U.S. Patent Application Publication No. 2007/0027135, is an unexpected benefit of great practical value, especially in view of the fact that formulation changes apparently have little effect on bioavailability of earlier generations of Bcl-2 protein family inhibitors such as ABT-737. Bioavailability in a rat model of ABT-737, formulated in 90% phosphatidylcholine+medium chain triglycerides 53/29 and 10% ethanol, was only 3.3%, not markedly different from that of other formulations tested.

Sufficient bioavailability of an ABT-263 composition is evidenced in some embodiments by one or both of

(a) an ABT-263 AUC₀₋₂₄ of at least about 20 μg·h/ml, and/or

(b) an ABT-263 Cₘₐₓ of at least about 2.5 μg/ml,

in a single-dose non-fasting human PK study at an ABT-263 free base equivalent dose of about 200 to about 400 mg.

Sufficient bioavailability of an ABT-263 composition is evidenced in other embodiments by a steady-state ABT-263 Cₐₘ, of about 1 to about 5 μg/ml and a steady-state ABT-263 Cₘₐₓ of about 3 to about 8 μg/ml in a non-fasting human pharmacokinetic study at a daily ABT-263 free base equivalent dose of about 200 to about 400 mg.

In particular embodiments, an ABT-263 composition is at least substantially bioequivalent to Formulation C as defined above.

The term “substantially bioequivalent” herein means exhibiting, in a human PK single- or multiple-dose study in fasting or non-fasting conditions, substantially equal Cₘₐₓ and substantially equal exposure measured as AUC, for example AUC₀₋₂₄, AUC₀₋₄₈ or AUC_{0-∞}. The compositions being compared for substantial bioequivalence should be administered at the same dose or doses, expressed as free base equivalent. If a multiple-dose study is used to draw the comparison, it is the steady-state values of Cₘₐₓ and AUC that are used. In the present context, Cₘₐₓ or AUC of a test composition is “substantially equal” if it is no less than 80% and no greater than 125% of the corresponding parameter in a reference composition (e.g., Formulation C).

Compositions embraced herein, including compositions described generally or with specificity herein, are useful for orally delivering a compound of Formula I, for example ABT-263, or a pharmaceutically acceptable salt thereof, to a subject. Accordingly, a method of the invention for delivering a compound of Formula I, for example ABT-263, or a pharmaceutically acceptable salt thereof, to a subject comprises orally administering a composition as described above.

The subject can be human or non-human (e.g., a farm, zoo, work or companion animal, or a laboratory animal used as a model) but in an important embodiment the subject is a human patient in need of the drug, for example to treat a disease characterized by apoptotic dysfunction and/or overexpression of an anti-apoptotic Bcl-2 family protein. A human subject can be male or female and of any age. The patient is typically an adult, but a method of the invention can be useful to treat a childhood cancer such as leukemia, for example acute lymphocytic leukemia, in a pediatric patient.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the bioavailability of the particular formulation, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of ABT-263 are generally about 25 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 μm, the average dosage interval is 8 hours (a 24-hour time span divided by 3). If the drug is formulated as a discrete dosage form such as a tablet or capsule, a plurality (e.g., 2 to 4) of dosage forms administered at one time is considered a unit dose for the purpose of defining the average dosage interval.

A daily dosage amount and dosage interval can, in some embodiments, be selected to maintain a plasma concentration of ABT-263 in a range of about 0.5 to about 10 μg/ml. Thus, during a course of ABT-263 therapy according to such embodiments, the steady-state peak plasma concentration (Cₘₐₓ) should in general not exceed about 10 μg/ml, and the steady-state trough plasma concentration (Cₘᵢₙ) should in general not fall below about 0.5 μg/ml. It will further be found desirable to select, within the ranges provided above, a daily dosage amount and average dosage interval effective to provide a Cₘₐₓ/Cₘₘ ratio not greater than about 5, for example not greater than about 3, at steady-state. It will be understood that longer dosage intervals will tend to result in greater Cₘₐₓ/Cₘₘ ratios. Illustratively, at steady-state, an ABT-263 Cₘₐₓ of about 3 to about 8 μg/ml and Cₘᵢₙ of about 1 to about 5 μg/ml can be targeted by the present method. Steady-state values of Cₘₐₓ and Cₘₘ can be established in a human PK study, for example conducted according to standard protocols including but not limited to those acceptable to a regulatory agency such as the U.S. Food and Drug Administration (FDA).

In the case of solid unit dosage forms, one to a small plurality of tablets or capsules can be swallowed whole, typically with the aid of water or other imbibable liquid to help the swallowing process. Optionally, tablets may be broken before swallowing and can be scored to facilitate even breakage.

As compositions of the present invention are believed to exhibit only a minor food effect, administration according to the present embodiment can be with or without food, i.e., in a non-fasting or fasting condition. It is generally preferred to administer the present compositions to a non-fasting patient.

### Method for Treating Disease

In still further embodiments of the invention, there is provided a method for treating a disease characterized by apoptotic dysfunction and/or overexpression of an anti-apoptotic Bcl-2 family protein, comprising administering to a subject having the disease a therapeutically effective amount of a compound of Formula I, for example ABT-263, or a pharmaceutically acceptable salt thereof, formulated in a composition as described herein.

Formulations of the present invention are suitable for use in monotherapy or in combination therapy, for example with other chemotherapeutics or with ionizing radiation. A particular advantage of the present invention is that it permits once-daily oral administration, a regimen which is convenient for the patient who is undergoing treatment with other orally administered drugs on a once-daily regimen. Oral administration is easily accomplished by the patient him/herself or by a caregiver in the patient's home; it is also a convenient route of administration for patients in a hospital or residential care setting.

Combination therapies illustratively include administration of a composition of the invention, for example such a composition comprising ABT-263, concomitantly with one or more of bortezomid, carboplatin, cisplatin, cyclophosphamide, dacarbazine, dexamethasone, docetaxel, doxorubicin, etoposide, fludarabine, hydroxydoxorubicin, irinotecan, paclitaxel, rapamycin, rituximab, vincristine and the like, for example with a polytherapy such as CHOP (cyclophosphamide+hydroxydoxorubicin+vincristine+prednisone), RCVP (rituximab+cyclophosphamide+vincristine+prednisone), R-CHOP (rituximab+CHOP) or DA-EPOCH-R dose-adjusted etoposide, prednisone, vincristine, cyclophosphamide, doxorubicin and rituximab).

A composition of the invention, for example such a composition comprising ABT-263, can be administered in combination therapy with one or more therapeutic agents that include, but are not limited to, angiogenesis inhibitors, antiproliferative agents, other apoptosis promoters (for example, Bcl-xL, Bcl-w and Bfl-1 inhibitors), activators of a death receptor pathway, BiTE (bi-specific T-cell engager) antibodies, dual variable domain binding proteins (DVDs), inhibitors of apoptosis proteins (IAPs), microRNAs, mitogen-activated extracellular signal-regulated kinase inhibitors, multivalent binding proteins, poly-ADP (adenosine diphosphate)-ribose polymerase (PARP) inhibitors, small inhibitory ribonucleic acids (siRNAs), kinase inhibitors, receptor tyrosine kinase inhibitors, aurora kinase inhibitors, polo-like kinase inhibitors, bcr-abl kinase inhibitors, growth factor inhibitors, COX-2 inhibitors, non-steroidal anti-inflammatory drugs (NSAIDs), antimitotic agents, alkylating agents, antimetabolites, intercalating antibiotics, platinum-containing chemotherapeutic agents, growth factor inhibitors, ionizing radiation, cell cycle inhibitors, enzymes, topoisomerase inhibitors, biologic response modifiers, immunologicals, antibodies, hormonal therapies, retinoids, deltoids, plant alkaloids, proteasome inhibitors, HSP-90 inhibitors, histone deacetylase (HDAC) inhibitors, purine analogs, pyrimidine analogs, MEK inhibitors, CDK inhibitors, ErbB2 receptor inhibitors, mTOR inhibitors as well as other antitumor agents.

Angiogenesis inhibitors include, but are not limited to, EGFR inhibitors, PDGFR inhibitors, VEGFR inhibitors, TIE2 inhibitors, IGF1R inhibitors, matrix metalloproteinase 2 (MMP-2) inhibitors, matrix metalloproteinase 9 (MMP-9) inhibitors and thrombospondin analogs.

Examples of EGFR inhibitors include, but are not limited to, gefitinib, erlotinib, cetuximab, EMD-7200, ABX-EGF, HR3, IgA antibodies, TP-38 (IVAX), EGFR fusion protein, EGF-vaccine, anti-EGFR immunoliposomes and lapatinib.

Examples of PDGFR inhibitors include, but are not limited to, CP-673451 and CP-868596.

Examples of VEGFR inhibitors include, but are not limited to, bevacizumab, sunitinib, sorafenib, CP-547632, axitinib, vandetanib, AEE788, AZD-2171, VEGF trap, vatalanib, pegaptanib, IM862, pazopanib, ABT-869 and angiozyme.

Bcl-2 family protein inhibitors other than ABT-263 include, but are not limited to, AT-101 ((−)gossypol), Genasense™ Bcl-2-targeting antisense oligonucleotide (G3139 or oblimersen), IPI-194, IPI-565, ABT-737, GX-070 (obatoclax) and the like.

Activators of a death receptor pathway include, but are not limited to, TRAIL, antibodies or other agents that target death receptors (e.g., DR4 and DR5) such as apomab, conatumumab, ETR2-ST01, GDC0145 (lexatumumab), HGS-1029, LBY-135, PRO-1762 and trastuzumab.

Examples of thrombospondin analogs include, but are not limited to, TSP-1, ABT-510, ABT-567 and ABT-898.

Examples of aurora kinase inhibitors include, but are not limited to, VX-680, AZD-1152 and MLN-8054.

An example of a polo-like kinase inhibitor includes, but is not limited to, BI-2536.

Examples of bcr-abl kinase inhibitors include, but are not limited to, imatinib and dasatinib.

Examples of platinum-containing agents include, but are not limited to, cisplatin, carboplatin, eptaplatin, lobaplatin, nedaplatin, oxaliplatin and satraplatin.

Examples of mTOR inhibitors include, but are not limited to, CCI-779, rapamycin, temsirolimus, everolimus, RAD001 and AP-23573.

Examples of HSP-90 inhibitors include, but are not limited to, geldanamycin, radicicol, 17-AAG, KOS-953, 17-DMAG, CNF-101, CNF-1010, 17-AAG-nab, NCS-683664, efungumab, CNF-2024, PU3, PU24FC1, VER-49009, IPI-504, SNX-2112 and STA-9090.

Examples of HDAC inhibitors include, but are not limited to, suberoylanilide hydroxamic acid (SAHA), MS-275, valproic acid, TSA, LAQ-824, trapoxin and depsipeptide.

Examples of MEK inhibitors include, but are not limited to, PD-325901, ARRY-142886, ARRY-438162 and PD-98059.

Examples of CDK inhibitors include, but are not limited to, flavopyridol, MCS-5A, CVT-2584, seliciclib ZK-304709, PHA-690509, BMI-1040, GPC-286199, BMS-387032, PD-332991 and AZD-5438.

Examples of COX-2 inhibitors include, but are not limited to, celecoxib, parecoxib, deracoxib, ABT-963, etoricoxib, lumiracoxib, BMS-347070, RS 57067, NS-398, valdecoxib, rofecoxib, SD-8381, 4-methyl-2-(3,4-dimethylphenyl)-1-(4-sulfamoylphenyl)-1H-pyrrole, T-614, JTE-522, S-2474, SVT-2016, CT-3 and SC-58125.

Examples of NSAIDs include, but are not limited to, salsalate, diflunisal, ibuprofen, ketoprofen, nabumetone, piroxicam, naproxen, diclofenac, indomethacin, sulindac, tolmetin, etodolac, ketorolac and oxaprozin.

Examples of ErbB2 receptor inhibitors include, but are not limited to, CP-724714, canertinib, trastuzumab, petuzumab, TAK-165, ionafamib, GW-282974, EKB-569, PI-166, dHER2, APC-8024, anti-HER/2neu bispecific antibody B7.her2IgG3 and HER2 trifunctional bispecific antibodies mAB AR-209 and mAB 2B-1.

Examples of alkylating agents include, but are not limited to, nitrogen mustard N-oxide, cyclophosphamide, ifosfamide, trofosfamide, chlorambucil, melphalan, busulfan, mitobronitol, carboquone, thiotepa, ranimustine, nimustine, Cloretazine™ (laromustine), AMD-473, altretamine, AP-5280, apaziquone, brostallicin, bendamustine, carmustine, estramustine, fotemustine, glufosfamide, KW-2170, mafosfamide, mitolactol, lomustine, treosulfan, dacarbazine and temozolomide.

Examples of antimetabolites include, but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, pemetrexed, gemcitabine, fludarabine, 5-azacitidine, capecitabine, cladribine, clofarabine, decitabine, eflornithine, ethenylcytidine, cytosine arabinoside, hydroxyurea, TS-1, melphalan, nelarabine, nolatrexed, disodium pemetrexed, pentostatin, pelitrexol, raltitrexed, triapine, trimetrexate, vidarabine, mycophenolic acid, ocfosfate, pentostatin, tiazofurin, ribavirin, EICAR, hydroxyurea and deferoxamine.

Examples of antibiotics include, but are not limited to, intercalating antibiotics, aclarubicin, actinomycin D, amrubicin, annamycin, adriamycin, bleomycin, daunorubicin, doxorubicin (including liposomal doxorubicin), elsamitrucin, epirubicin, glarubicin, idarubicin, mitomycin C, nemorubicin, neocarzinostatin, peplomycin, pirarubicin, rebeccamycin, stimalamer, streptozocin, valrubicin, zinostatin and combinations thereof.

Examples of topoisomerase inhibiting agents include, but are not limited to, aclarubicin, amonafide, belotecan, camptothecin, 10-hydroxycamptothecin, 9-amino-camptothecin, amsacrine, dexrazoxane, diflomotecan, irinotecan HCl, edotecarin, epirubicin, etoposide, exatecan, becatecarin, gimatecan, lurtotecan, orathecin, BN-80915, mitoxantrone, pirarbucin, pixantrone, rubitecan, sobuzoxane, SN-38, tafluposide and topotecan.

Examples of antibodies include, but are not limited to, rituximab, cetuximab, bevacizumab, trastuzumab, CD40-specific antibodies and IGF1R-specific antibodies, chTNT-1/B, denosumab, edrecolomab, WX G250, zanolimumab, lintuzumab and ticilimumab.

Examples of hormonal therapies include, but are not limited to, sevelamer carbonate, rilostane, luteinizing hormone releasing hormone, modrastane, exemestane, leuprolide acetate, buserelin, cetrorelix, deslorelin, histrelin, anastrozole, fosrelin, goserelin, degarelix, doxercalciferol, fadrozole, formestane, tamoxifen, arzoxifene, bicalutamide, abarelix, triptorelin, finasteride, fulvestrant, toremifene, raloxifene, trilostane, lasofoxifene, letrozole, flutamide, megesterol, mifepristone, nilutamide, dexamethasone, prednisone and other glucocorticoids.

Examples of retinoids or deltoids include, but are not limited to, seocalcitol, lexacalcitol, fenretinide, aliretinoin, tretinoin, bexarotene and LGD-1550.

Examples of plant alkaloids include, but are not limited to, vincristine, vinblastine, vindesine and vinorelbine.

Examples of proteasome inhibitors include, but are not limited to, bortezomib, MG-132, NPI-0052 and PR-171.

Examples of immunologicals include, but are not limited to, interferons and numerous other immune-enhancing agents. Interferons include interferon alpha, interferon alpha-2a, interferon alpha-2b, interferon beta, interferon gamma-1a, interferon gamma-1b, interferon gamma-n1 and combinations thereof. Other agents include filgrastim, lentinan, sizofilan, BCG live, ubenimex, WF-10 (tetrachlorodecaoxide or TCDO), aldesleukin, alemtuzumab, BAM-002, dacarbazine, daclizumab, denileukin, gemtuzumab ozogamicin, ibritumomab, imiquimod, lenograstim, melanoma vaccine, molgramostim, sargaramostim, tasonermin, tecleukin, thymalasin, tositumomab, Virulizin™ immunotherapeutic of Lorus Pharmaceuticals, Z-100 (specific substance of Maruyama or SSM), Zevalin™ (⁹⁰Y-ibritumomab tiuxetan), epratuzumab, mitumomab, oregovomab, pemtumomab, Provenge™ (sipuleucel-T), teceleukin, Therocys™ (Bacillus Calmette-Guerin), cytotoxic lymphocyte antigen 4 (CTLA4) antibodies and agents capable of blocking CTLA4 such as MDX-010.

Examples of biological response modifiers are agents that modify defense mechanisms of living organisms or biological responses, such as survival, growth, or differentiation of tissue cells to direct them to have anti-tumor activity. Such agents include, but are not limited to, krestin, lentinan, sizofuran, picibanil, PF-3512676 and ubenimex.

Examples of pyrimidine analogs include, but are not limited to, 5-fluorouracil, floxuridine, doxifluridine, raltitrexed, cytarabine, cytosine arabinoside, fludarabine, triacetyluridine, troxacitabine and gemcitabine.

Examples of purine analogs include, but are not limited to, mercaptopurine and thioguanine.

Examples of antimitotic agents include, but are not limited to, N-(2-((4-hydroxyphenyl)amino)pyridin-3-yl)-4-methoxybenzenesulfonamide, paclitaxel, docetaxel, larotaxel, epothilone D, PNU-100940, batabulin, ixabepilone, patupilone, XRP-9881, vinflunine and ZK-EPO (synthetic epothilone).

Examples of radiotherapy include, but are not limited to, external beam radiotherapy (XBRT), teletherapy, brachytherapy, sealed-source radiotherapy and unsealed-source radiotherapy.

BiTE antibodies are bi-specific antibodies that direct T-cells to attack cancer cells by simultaneously binding the two cells. The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (Sutton et al. (1997) J. Immunol. 158:5783-5790).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mcl-1 have been shown to enhance the activity of ABT-263 (Tse et al. (2008), supra, and references therein).

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy-chain DVD polypeptides and two light-chain DVD polypeptides are referred to as DVD Ig's. Each half of a DVD Ig comprises a heavy-chain DVD polypeptide, a light-chain DVD polypeptide, and two antigen binding sites. Each binding site comprises a heavy-chain variable domain and a light-chain variable domain with a total of 6 CDRs involved in antigen binding per antigen binding site.

PARP inhibitors include, but are not limited to, ABT-888, olaparib, KU-59436, AZD-2281, AG-014699, BSI-201, BGP-15, INO-1001, ONO-2231 and the like.

Additionally or alternatively, a composition of the invention, for example such a composition comprising ABT-263, can be administered in combination therapy with one or more antitumor agents selected from ABT-100, N-acetylcolchinol-O-phosphate, acitretin, AE-941, aglycon protopanaxadiol, arglabin, arsenic trioxide, AS04 adjuvant-adsorbed HPV vaccine, L-asparaginase, atamestane, atrasentan, AVE-8062, bosentan, canfosfamide, Canvaxin™, catumaxomab, CeaVac™, celmoleukin, combrestatin A4P, contusugene ladenovec, Cotara™, cyproterone, deoxycoformycin, dexrazoxane, N,N-diethyl-2-(4-(phenylmethyl)phenoxy)ethanamine, 5,6-dimethylxanthenone-4-acetic acid, docosahexaenoic acid/paclitaxel, discodermolide, efaproxiral, enzastaurin, epothilone B, ethynyluracil, exisulind, falimarev, Gastrimmune™, GMK vaccine, GVA™, halofuginone, histamine, hydroxycarbamide, ibandronic acid, ibritumomab tiuxetan, IL-13-PE38, inalimarev, interleukin 4, KSB-311, lanreotide, lenalidomide, lonafarnib, lovastatin, 5,10-methylenetetrahydrofolate, mifamurtide, miltefosine, motexafin, oblimersen, OncoVAX™ Osidem™, paclitaxel albumin-stabilized nanoparticle, paclitaxel poliglumex, pamidronate, panitumumab, peginterferon alia, pegaspargase, phenoxodiol, poly(I)-poly(C12U), procarbazine, ranpirnase, rebimastat, recombinant quadrivalent HPV vaccine, squalamine, staurosporine, STn-KLH vaccine, T4 endonuclase V, tazarotene, 6,6′,7,12-tetramethoxy-2,2′-dimethyl-1β-berbaman, thalidomide, TNFerade™, ¹³¹I-tositumomab, trabectedin, triazone, tumor necrosis factor, Ukrain™, vaccinia-MUC-1 vaccine, L-valine-L-boroproline, Vitaxin™, vitespen, zoledronic acid and zorubicin.

In one embodiment, a composition of the invention, for example such a composition comprising ABT-263, is administered in a therapeutically effective amount to a subject in need thereof to treat a disease during which is overexpressed one or more of antiapoptotic Bcl-2 protein, antiapoptotic Bcl-X_{L} protein and antiapoptotic Bcl-w protein.

In another embodiment, a composition of the invention, for example such a composition comprising ABT-263, is administered in a therapeutically effective amount to a subject in need thereof to treat a disease of abnormal cell growth and/or dysregulated apoptosis.

Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In a more particular embodiment, a composition of the invention, for example such a composition comprising ABT-263, is administered in a therapeutically effective amount to a subject in need thereof to treat bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer or spleen cancer.

According to any of these embodiments, the composition can be administered in monotherapy or in combination therapy with one or more additional therapeutic agents.

For example, a method for treating mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof in a subject comprises administering to the subject therapeutically effective amounts of (a) a composition of the invention, for example such a composition comprising ABT-263, and (b) one or more of etoposide, vincristine, CHOP, rituximab, rapamycin, R-CHOP, RCVP, DA-EPOCH-R or bortezomib.

In particular embodiments, a composition of the invention, for example such a composition comprising ABT-263, is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of a lymphoid malignancy such as B-cell lymphoma or non-Hodgkin's lymphoma.

In other particular embodiments, a composition of the invention, for example such a composition comprising ABT-263, is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of chronic lymphocytic leukemia or acute lymphocytic leukemia.

The present invention also provides a method for maintaining in bloodstream of a human cancer patient a therapeutically effective plasma concentration of ABT-263 and/or one or more metabolites thereof, comprising administering to the subject an ABT-263 composition as described herein, in a dosage amount of about 50 to about 500 mg ABT-263 free base equivalent per day, at an average dosage interval of about 3 hours to about 7 days.

What constitutes a therapeutically effective plasma concentration depends inter alia on the particular cancer present in the patient, the stage, severity and aggressiveness of the cancer, and the outcome sought (e.g., stabilization, reduction in tumor growth, tumor shrinkage, reduced risk of metastasis, etc.). It is strongly preferred that, while the plasma concentration is sufficient to provide benefit in terms of treating the cancer, it should not be sufficient to provoke an adverse side-effect to an unacceptable or intolerable degree.

Further information of relevance to the present invention is available in a recently published article by Tse et al. (2008) Cancer Res. 68:3421-3428 and supplementary data thereto available at Cancer Research Online (cancerres.aacrjournals.org/). This article and its supplementary data are incorporated in their entirety herein by reference.

### EXAMPLES

The following examples are illustrative of the invention or of problems overcome by the invention, but are not to be construed as limiting. Characterization of a particular embodiment as unfavorable or not selected for preparation of a prototype formulation does not necessarily mean that such embodiment is totally inoperative or outside the scope of the invention. One of skill in the art, based on the full disclosure herein, can prepare acceptable formulations even using ingredients shown herein to be suboptimal.

Trademarked ingredients used in the examples, which can be substituted with comparable ingredients from other suppliers, include:
Avicel 101™ and Avicel 102™ of FMC: microcrystalline cellulose;
Imwitor 742™ of Sasol: caprylic/capric mono- and diglycerides;
Miglyol 810™ of Sasol: caprylic/capric triglycerides;
Capmul MCM™ of Abitec: glyceryl caprylate/caprate;
Capmul PG-8™ of Abitec: propylene glycol monocaprylate;
Capmul PG12™ of Abitec: propylene glycol monolaurate;
Captex 300™ of Abitec: caprylic/capric triglycerides;
Cremophor EL™ of BASF: polyoxyethylene (35) castor oil;
Cremophor RH40™ of BASF: polyoxyethylene (40) hydrogenated castor oil;
Crillet 4HP™ of Croda: polysorbate 80 having low peroxide value;
Gelucire 44/14™ of Gattefossé: polyoxyethylene glyceryl laurate;
Phosal 53 MCT™ of Phospholipid GmbH: blend containing not less than 53% phosphatidylcholine, not more than 6% lysophosphatidylcholine, about 29% medium chain triglycerides, 3-6% ethanol, about 3% mono- and diglycerides from sunflower oil, about 2% oleic acid, and about 0.2% ascorbyl palmitate;
Plurol Oleique CC497™ of Gattefossé: polyglyceryl oleate;
ProSolv HD 90™ of JRS Pharma: silicified microcrystalline cellulose;
Labrafil M 1944 CS™ of Gattefossé: polyoxyethylene glyceryl monooleate;
Labrafil M 2125 CS™ of Gattefossé: polyoxyethylene glyceryl linoleate;
Labrasol™ of Gattefossé: polyoxyethylene glyceryl caprylate/caprate;
Lauroglycol 90™ of Gattefossé: propylene glycol monolaurate;
Lipoid S75™ MCT (prepared from Lipoid S75™ of Lipoid GmbH): blend containing not less than 20% phosphatidylcholine, 2-4% phosphatidylethanolamine, not more than 1.5% lysophosphatidylcholine, and 67-73% medium-chain triglycerides;
Span™ 20 of Croda International PLC: sorbitan monolaurate;
Starch 1500™ of Colorcon: pregelatinized starch;
Tween™ 20 of Uniqema: polysorbate 20;
Tween™ 80 of Uniqema: polysorbate 80;
Vitamin E TPGS™: α-tocopheryl polyethylene glycol (1000) succinate (TPGS).

All ABT-263 amounts, including concentrations and doses, given in the examples are expressed as free base equivalent doses unless expressly stated otherwise. Where ABT-263 is administered as bis-HCl salt, 1.076 mg ABT-263 bis-HCl provides 1 mg ABT-263 free base equivalent.

### Example 1

### Solubility of ABT-263 Parent and bis-HCl Salt in Lipid Solvents

Solubility of ABT-263 parent (free base, crystalline Form I) and ABT-263 bis-HCl salt was tested in a variety of lipid solvents and solvent mixtures in ambient conditions. “PE-91” is Phosal 53 MCT™+ethanol, 9:1 by volume. “LOT-343” is Labrafil M 1944 CS™+oleic acid+Tween 80™, 30:40:30 by weight.

Solubility data are presented in Table 4. In some cases, indicated in Table 4 by an asterisk (*), solubility was initially high but precipitation occurred upon standing.

TABLE 4
Solubility (mg/g) of ABT-263 parent and bis-HCl salt in lipid solvents

| Solvent | Parent (Form I) | bis-HCl salt |
|---|---|---|
| corn oil | <86 | <104 |
| sesame oil | <75 | <80 |
| castor oil | * | >78.8 |
| Miglyol 810 ™ | <76 | <84 |
| Lipoid S75 ™ MCT | 150-200 | 48.9 |
| Phosal 53 MCT ™ | >300 | n.d. |
| oleic acid | >514 | <498 |
| Imwitor 742 ™ | * | >245 |
| Capmul MCM ™ | * | >321 |
| Capmul PG-8 ™ | * | <43 |
| Capmul PG-12 ™ | * | <39 |
| Captex 300 ™ | * | <52 |
| Labrafil M 1944 CS ™ | >265 | <45 |
| Labrafil M 2125 CS ™ | >290 | <44 |
| PEG-400 | >200 | >278 |
| propylene glycol | * | >337 |
| Tween ™ 20 | >256 | >176 |
| Tween ™ 80 | >256 | >125 |
| Labrasol ™ | >242 | >292 |
| Cremophor RH40 ™ | >226 | n.d. |
| poloxamer 124 | >231 | <41 |
| PE-91 | >250 | 89 |
| LOT-343 | >479 | n.d. |

n.d. not determined

### Example 2

### Miscibility of Ternary Excipient Systems with ABT-263 Parent and bis-HCl Salt

Ternary systems consisting of two solvents and a surfactant were evaluated for miscibility and drug solubility using 20% by weight ABT-263 free base or 10% by weight ABT-263 bis-HCl salt. Solvents evaluated included Labrafil M 1944 CS™, Imwitor 742™ oleic acid, Capmul PG-8™, Capmul PG-12™, Lauroglycol 90™ and Phosal 53 MCT™. Surfactants evaluated included Tween™ 80, Cremophor RH40™, Gelucire 44/14™ and Labrasol™. Data are presented in Table 5.

TABLE 5
Miscibility of ternary systems and solubility of ABT-263 parent
and bis-HCl salt

| | | Miscibility | ABT-263 solubility | |
|---|---|---|---|---|
| | % by | of | | 20% free |
| Ternary system | weight | excipients | 10% salt | base |
| Labrafil M 1944 CS ™ | 30:45:25 | ✓ | ✓ | X |
| Imwitor 742 ™ | 40:35:25 | ✓ | ✓ | X |
| Tween 80 ™ | 30:40:30 | ✓ | ✓ | X |
| (LIT systems) | 40:30:30 | ✓ | ✓ | X |
| Labrafil M 1944 CS ™ | 30:45:25 | ✓ | ✓ | ✓ |
| oleic acid | 40:35:25 | ✓ | ✓ | ✓ |
| Tween 80 ™ | 30:40:30 | ✓ | ✓ | ✓ |
| (LOT systems) | 40:30:30 | ✓ | ✓ | ✓ |
| Capmul PG-8 ™ | 45:30:25 | ✓ | X | X |
| Labrafil M 1944 CS ™ | 35:40:25 | ✓ | X | X |
| Tween 80 ™ | 40:30:30 | ✓ | X | X |
| (C8LT systems) | 30:40:30 | ✓ | X | X |
| Capmul PG-12 ™ | 45:30:25 | ✓ | ✓ | ✓ |
| Labrafil M 1944 CS ™ | 35:40:25 | ✓ | ✓ | ✓ |
| Tween 80 ™ | 40:30:30 | ✓ | ✓ | ✓ |
| (C12LT systems) | 30:40:30 | ✓ | ✓ | ✓ |
| Imwitor 742 ™ | 45:30:25 | X | N/A (vehicle | |
| | | | not miscible) | |
| Labrafil M 1944 CS ™ | 35:40:25 | X | N/A (vehicle | |
| | | | not miscible) | |
| Cremophor RH40 ™ | 40:30:30 | X | N/A (vehicle | |
| | | | not miscible) | |
| (ILC systems) | 30:40:30 | X | N/A (vehicle | |
| | | | not miscible) | |
| | 60:30:10 | ✓ | ✓ | X |
| | 50:40:10 | ✓ | ✓ | X |
| | 50:30:20 | ✓ | ✓ | X |
| | 40:40:20 | ✓ | ✓ | X |
| Labrafil M 1944 CS ™ | 30:45:25 | X | N/A (vehicle | |
| | | | not miscible) | |
| oleic acid | 40:35:25 | X | N/A (vehicle | |
| | | | not miscible) | |
| Cremophor RH40 ™ | 30:40:30 | X | N/A (vehicle | |
| | | | not miscible) | |
| (LOC systems) | 40:30:30 | X | N/A (vehicle | |
| | | | not miscible) | |
| | 30:60:10 | ✓ | ✓ | ✓ |
| | 40:50:10 | ✓ | ✓ | ✓ |
| | 30:50:20 | X | N/A (vehicle | |
| | | | not miscible) | |
| | 40:40:20 | X | N/A (vehicle | |
| | | | not miscible) | |
| Capmul PG-8 ™ | 45:30:25 | X | N/A (vehicle | |
| | | | not miscible) | |
| Labrafil M 1944 CS ™ | 35:40:25 | X | N/A (vehicle | |
| | | | not miscible) | |
| Cremophor RH40 ™ | 40:30:30 | X | N/A (vehicle | |
| | | | not miscible) | |
| (C8LC systems) | 30:40:30 | X | N/A (vehicle | |
| | | | not miscible) | |
| | 60:30:10 | ✓ | X | X |
| | 50:40:10 | ✓ | X | X |
| | 50:30:20 | ✓ | X | X |
| | 40:40:20 | ✓ | X | X |
| Capmul PG-12 ™ | 45:30:25 | X | N/A (vehicle | |
| | | | not miscible) | |
| Labrafil M 1944 CS ™ | 35:40:25 | X | N/A (vehicle | |
| | | | not miscible) | |
| Cremophor RH40 ™ | 40:30:30 | X | N/A (vehicle | |
| | | | not miscible) | |
| (C12LC systems) | 30:40:30 | X | N/A (vehicle | |
| | | | not miscible) | |
| Lauroglycol 90 ™ | 45:30:25 | ✓ | ✓ | ✓ |
| Labrafil M 1944 CS ™ | 35:40:25 | X | N/A (vehicle | |
| | | | not miscible) | |
| Cremophor RH40 ™ | 40:30:30 | X | N/A (vehicle | |
| | | | not miscible) | |
| (LLC systems) | 30:40:30 | X | N/A (vehicle | |
| | | | not miscible) | |
| Imwitor 742 ™ | 60:30:10 | X | N/A (vehicle | |
| | | | not miscible) | |
| Labrafil M 1944 CS ™ | 50:40:10 | X | N/A (vehicle | |
| | | | not miscible) | |
| Gelucire 44/14 ™ | 50:30:20 | X | N/A (vehicle | |
| | | | not miscible) | |
| (ILG systems) | 40:40:20 | X | N/A (vehicle | |
| | | | not miscible) | |
| oleic acid | 60:30:10 | X | N/A (vehicle | |
| | | | not miscible) | |
| Labrafil M 1944 CS ™ | 50:40:10 | X | N/A (vehicle | |
| | | | not miscible) | |
| Gelucire 44/14 ™ | 50:30:20 | X | N/A (vehicle | |
| | | | not miscible) | |
| (OLG systems) | 40:40:20 | X | N/A (vehicle | |
| | | | not miscible) | |
| Capmul PG-8 ™ | 60:30:10 | X | N/A (vehicle | |
| | | | not miscible) | |
| Labrafil M 1944 CS ™ | 50:40:10 | X | N/A (vehicle | |
| | | | not miscible) | |
| Gelucire 44/14 | 50:30:20 | X | N/A (vehicle | |
| | | | not miscible) | |
| (C8LG systems) | 40:40:20 | X | N/A (vehicle | |
| | | | not miscible) | |
| Lauroglycol 90 ™ | 60:30:10 | X | N/A (vehicle | |
| | | | not miscible) | |
| Labrafil M 1944 CS ™ | 50:40:10 | X | N/A (vehicle | |
| | | | not miscible) | |
| Gelucire 44/14 ™ | 50:30:20 | X | N/A (vehicle | |
| | | | not miscible) | |
| (LLG systems) | 40:40:20 | X | N/A (vehicle | |
| | | | not miscible) | |
| Imwitor 742 ™ | 60:30:10 | ✓ | ✓ | X |
| Labrafil M 1944 CS ™ | 50:40:10 | ✓ | ✓ | X |
| Labrasol ™ | 50:30:20 | ✓ | ✓ | X |
| (ILL systems) | 40:40:20 | ✓ | ✓ | X |
| oleic acid | 60:30:10 | ✓ | ✓ | ✓ |
| Labrafil M 1944 CS ™ | 50:40:10 | ✓ | ✓ | ✓ |
| Labrasol ™ | 50:30:20 | ✓ | ✓ | ✓ |
| (OLL systems) | 40:40:20 | ✓ | ✓ | ✓ |
| Capmul PG-8 | 60:30:10 | ✓ | X | X |
| Labrafil M 1944 CS ™ | 50:40:10 | ✓ | X | X |
| Labrasol ™ | 50:30:20 | ✓ | X | X |
| (C8LL systems) | 40:40:20 | ✓ | ✓ | ✓ |
| Lauroglycol 90 ™ | 60:30:10 | ✓ | ✓ | X |
| Labrafil M 1944 CS ™ | 50:40:10 | ✓ | ✓ | X |
| Labrasol ™ | 50:30:20 | ✓ | ✓ | ✓ |
| (LLL systems) | 40:40:20 | ✓ | ✓ | ✓ |

All ternary excipient systems tested containing 10-20% Gelucire 44/14™ exhibited immiscibility. Most systems tested containing greater than 20% Cremophor RH40™ also showed immiscibility. Only in certain systems where the excipients were miscible was ABT-263 in free base or bis-HCl salt form soluble at the concentrations tested.

Data for further ternary systems containing phosphatidylcholine-based excipients are presented in Example 8, Tables 11 and 12.

### Example 3

### Chemical Stability of ABT-263 Free Base and bis-HCl Salt in Lipid Solution

Preliminary stability studies were conducted to allow a side-by-side comparison between lipid solutions of ABT-263 in bis-HCl salt and free base form. ABT-263 was dissolved in two separate sets of lipid vehicles, Phosal 53 MCT™/ethanol (9:1 by volume; “PE-91”) and Labrafil M 1944 CS™/oleic acid/Tween 80™ (30:40:30 by weight; “LOT-343”). No antioxidant was included, nor was headspace nitrogen purging performed. After aging of samples at 40° C. (stress condition) for up to 3 weeks, analysis of total sulfoxides indicated that free base was significantly more stable than bis-HCl salt in the solutions tested (Table 6). Total degradant levels also showed a similar trend (data not shown). The increase in degradant level was accompanied by color change. The bis-HCl salt solutions upon aging showed pronounced color darkening whereas the free base solutions exhibited very little color change.

TABLE 6
Sulfoxide formation in lipid solutions of ABT-263 free
base and bis-HCl salt

| | % w/w total sulfoxides | | | |
|---|---|---|---|---|
| | Solution A | | Solution B | |
| Time | free base | bis-HCl salt | free base | bis-HCl salt |
| (weeks) | 25 mg/ml | 25 mg/ml | 100 mg/ml | 100 mg/ml |
| 0 | 0.05 | 0.07 | 2.49 | 2.24 |
| 1 | 0.27 | 0.79 | 3.70 | 7.15 |
| 2 | 0.53 | 1.90 | 4.11 | 37.52 |
| 3 | 0.84 | 3.44 | no data | no data |

### Example 4

### Chemical Stability of ABT-263 Free Base in Various Lipid Solutions

The chemical stability of the ABT-263 free base in solution in various lipid excipients was assessed by conducting a two-week stress test at 40° C., without antioxidant or nitrogen purging. Results are presented in Table 7.

TABLE 7
Sulfoxide formation in lipid solutions of ABT-263 free base

| | | % w/w total | | |
|---|---|---|---|---|
| | Concentration | sulfoxides* | | |
| Lipid solvent | (mg/g) | Initial | 1 week | 2 weeks |
| Lipoid S75 ™ MCT | 100 | 0.21 | 0.33 | 0.51 |
| Imwitor 742 ™ | 25** | 0.25 | 0.20 | 0.14 |
| Capmul PG-8 ™ | 25** | 0.21 | 0.25 | 0.19 |
| Tween 80 ™ | 100 | 0.20 | 0.59 | 0.84 |
| Crillet 4HP ™ | 100 | 0.18 | 0.44 | 0.64 |
| Plurol Oleique | 50** | 0.31 | 2.41 | 6.26 |
| CC497 ™/Lipoid | | | | |
| S75 ™ MCT 50:50 v/v | | | | |
| Labrafil M 1944 CS ™ | 100 | 0.30 | 5.86 | 9.16 |
| oleic acid (super-refined) | 100 | 0.04 | 0.18 | 0.29 |
| Phosal 53 MCT ™/ | 50 | n.d. | 0.14 | 0.18 |
| ethanol 9:1 v/v | | | | |

*sulfoxide was analyzed as peak % relative to that of ABT-263
**lower concentration was used due to low drug solubility in the lipid vehicle
n.d. not detectable

The following can be summarized from the above study.
Very little or only slight growth of sulfoxides was seen in phosphatidylcholine-based lipid excipients such as Phosal 53 MCT™ or Lipoid S75™ MCT.
Very little or only slight growth of sulfoxides was seen in Imwitor 742™, Capmul PG-8™ and oleic acid (super-refined grade).
Moderate sulfoxide growth was seen in Tween 80™. The degradation was slowed down when a purer grade of polysorbate 80 (Crillet 4HP™) was used.
Labrafil M 1944 CS™ and Plurol Oleique CC497™ were both associated with significant degradation of the ABT-263. Both these excipients contain oleic acid in their structure, and the unsaturated nature of oleic acid is known to promote oxidative reaction. This may be the reason for the chemical instability of the drug in these excipients.

### Example 5

### Chemical Stability of ABT-263 Free Base in Ternary Lipid Solution Systems

Although ABT-263 appeared to be stable in super-refined oleic acid during the two-week stressed test of Example 4, a subsequent test using multicomponent vehicles showed that drug solutions containing oleic acid led to color change upon standing. A comparative storage study was conducted at ambient temperature using solutions of ABT-263 in Imwitor 742™/oleic acid/Tween 80™ (30:40:30 by weight; “IOT-343”) and Imwitor 742™/Phosal 53 MCT™/Tween 80™ (40:40:20 by weight; “IPT-442”). The IOT-343 vehicle itself was colorless, and adding ABT-263 free base at 10% by weight to the vehicle only made it very slightly yellow-hued, but the color of the resulting ABT-263 solution darkened significantly upon storage. This was in contrast to a solution of ABT-263 free base at 10% by weight in IPT-442 solution, which had a yellow colored vehicle to begin with, but only darkened slightly upon storage. HPLC analysis for the two drug solutions after storage at ambient conditions for 3 months confirmed that the color change correlated to degradation (total sulfoxide levels were 1.3% for the IOT-343 system and 0.5% for the IPT-442 system). Therefore, oleic acid was excluded from lipid excipients to be used for ABT-263 liquid-filled capsule formulation.

Further stress testing on ABT-263 free base lipid solutions using different ternary lipid combinations showed that Labrafil M 1944 CS™ was also associated with significant oxidative degradation of ABT-263. As shown by results from a three-week stress test presented in Table 8, formulations containing Labrafil M 1944 CS™ showed significant sulfoxide growth upon storage at 40° C. without antioxidant or nitrogen purging. On the other hand, an Imwitor 742™/Phosal 53 MCT™/Tween 80™ (20:50:30 by weight; “IPT-253”) solution of ABT-263 which had neither oleic acid nor Labrafil M 1944 CS™ showed much enhanced chemical stability compared to the other formulations tested, namely Labrafil M 1944 CS™/oleic acid/Tween 80™ (30:40:30 by weight; “LOT-343”) and Labrafil M 1944 CST™/Imwitor 742™/Tween 80™ (40:30:30 by weight; “LIT-433”). Therefore, both Labrafil M 1944 CS™ as well as oleic acid was excluded from lipid excipients to be used for ABT-263 liquid-filled capsule formulation.

TABLE 8
Sulfoxide formation in ternary lipid solutions of ABT-263 free base

| Ternary lipid | Concentration | % w/w total sulfoxides* | | | |
|---|---|---|---|---|---|
| solvent system | (mg/g) | Initial | 1 week | 2 weeks | 3 weeks |
| LOT-343 | 100 | 2.49 | 3.70 | 4.11 | no data |
| LIT-433 | 100 | 0.21 | 3.20 | 5.13 | no data |
| LIT-433 | 150 | 0.23 | 2.28 | 3.61 | 3.80 |
| IPT-253 | 150 | n.d. | 0.26 | 0.47 | 0.56 |

*sulfoxide was analyzed as peak % relative to that of ABT-263
n.d. not detectable

### Example 6

### Antioxidant Testing for ABT-263 Free Base in Lipid Solution Systems

The effectiveness of different antioxidants in inhibiting oxidative degradation was evaluated in lipid solutions containing ABT-263 free base at 100 mg/g in two different lipid solution systems: (1) Lipoid S75™ MCT and (2) a ternary lipid system (LIT-433; see above). The latter was purposely chosen as a system promoting significant degradation in a short time, as an antioxidant screen. Sulfoxide formation during the two-week stress test at 40° C. with nitrogen purging is shown in Table 9.

TABLE 9
Effect of antioxidants on sulfoxide formation in solutions of ABT-263 free base

| | | % w/w total sulfoxides* | | | | | |
|---|---|---|---|---|---|---|---|
| | Antioxidant | In Lipoid S75 ™ MCT | | | In LIT-433 | | |
| Antioxidant | concentration | Initial | 1 week | 2 weeks | Initial | 1 week | 2 weeks |
| none | | 0.06 | 0.42 | 0.68 | 0.21 | 3.20 | 5.13 |
| ascorbyl palmitate | 100% molar** | n.d. | n.d. | n.d. | 0.31 | 1.37 | 2.07 |
| BHA | 100% molar** | 0.13 | 0.26 | 0.30 | 0.43 | 2.25 | 3.66 |
| BHT | 100% molar** | 0.08 | 0.17 | 0.27 | 0.37 | 2.07 | 3.40 |
| Na metabisulfite*** | 0.1% (w/w) | cloudy solution | | | 0.18 | 1.95 | 3.07 |
| Na thiosulfate*** | 0.1% (w/w) | cloudy solution | | | 0.18 | 2.64 | 4.31 |
| thioglycerol | 100% molar** | 0.08 | 0.09 | 0.13 | 0.33 | 0.50 | 0.56 |
| α-tocopherols | 145% molar** | 0.20 | 0.27 | 0.50 | 0.41 | 3.99 | 9.23 |

n.d. not determined (ascorbyl palmitate could not be dissolved at 100% relative molar concentration in this solvent)
*sulfoxide was analyzed as peak % relative to that of ABT-263
**molar concentration relative to ABT-263
***an aqueous stock solution of 15% w/v was prepared for antioxidant addition.

ABT-263 free base degraded to a much lesser extent in the Lipoid S75™ MCT vehicle than in the LIT-433 vehicle system. Thioglycerol provided effective inhibition of drug oxidation in both vehicle systems. In the LIT-433 vehicle system, ascorbyl palmitate, butylated hydroxyanisole (BHA), butylated hydroxytoluene (BHT), sodium metabisulfite and sodium thiosulfate inhibited oxidative degradation to some extent at the concentrations tested, but α-tocopherols were ineffective. It is noted that the concentrations of sodium metabisulfite and sodium thiosulfate were very much lower than those providing molar equivalence to ABT-263. Even at the low concentrations used, the addition of water with these antioxidants led to cloudy solutions. The concentrations of ascorbyl palmitate, BHA and BHT were much higher than typically used for antioxidant purposes.

### Example 7

### BHA as an Antioxidant for ABT-263 Free Base in Ternary Lipid Solution Systems

Due to its favorable lipophilic nature and wide use in lipid system as an antioxidant, the antioxidant effectiveness of BHA was tested, at a concentration more typical for BHA, in two additional ternary vehicle systems, IPT-253 and LIT-433, containing ABT-263 at 150 mg/g. Testing was done in stress conditions at 40° C. without nitrogen purging. As shown in Table 10, in both systems, addition of 0.2% w/w BHA did not provide any inhibition of sulfoxide formation. It was concluded that free-radical-scavenger types of antioxidant such as BHA and BHT do not appear to be useful in protecting ABT-263 from oxidative degradation in lipid solutions.

TABLE 10
Effect of BHA on sulfoxide formation in
solutions of ABT-263 free base

| Ternary | | % w/w total sulfoxides | | | | |
|---|---|---|---|---|---|---|
| system | Antioxidant | Initial | 1 week | 2 weeks | 3 weeks | 4 weeks |
| IPT-253 | none | n.d. | 0.26 | 0.47 | 0.56 | 0.67 |
| | 0.2% w/w BHA | 0.06 | 0.29 | 0.49 | 0.58 | 0.68 |
| LIT-433 | none | 0.23 | 2.28 | 3.61 | 3.86 | 4.19 |
| | 0.2% w/w BHA | 0.24 | 2.22 | 3.54 | 3.80 | 4.19 |

n.d. not detectable

### Example 8

### Phospholipid Solution Systems for ABT-263 Free Base

Based on the above studies, the phosphatidylcholine-containing excipients Phosal 53 MCT™ and Lipoid S75™ MCT were concluded to provide good chemical stability and drug solubility for ABT-263 free base. However, these pre-blended excipients are not suitable for use alone as a vehicle for an ABT-263 liquid-filled capsule, due to either high viscosity (Phosal 53 MCT™) or insufficient drug solubility (Lipoid S75™ MCT). Polysorbate 80 could be used to enhance drug solubility in the vehicle. Excipients such as Capmul PG8™ or Imwitor 742™ could be used to reduce viscosity of the lipid solution. Both were shown to be chemically compatible with ABT-263. Imwitor 742™ was preferred over Capmul PG8™ based on previous experience in FDA approved drug products.

Consequently, in developing a prototype liquid-filled capsule, attention focused on excipients such as Phosal 53 MCT™, Lipoid S75™ MCT, polysorbate 80 (the purer forms such as Crillet 4HP™ and super-refined Tween 80™ being preferred) and Imwitor 742™

Two ternary lipid vehicle systems containing either Imwitor 742™/Phosal 53 MCT™/Tween 80™ (abbreviated as “IPT”) systems or Imwitor 742™/Lipoid S75™ MCT/Tween 80™ (abbreviated as “IST”) systems at various excipient ratios were investigated in a screen for prototype capsule formulations. The level of Imwitor 742™ in the ternary blend was limited to no more than 40%, and the level of polysorbate 80 to no more than 20%. The three-digit suffix following “IPT” or “IST” refers to the respective percentages of the three excipient ingredients, in each case omitting the final zero.

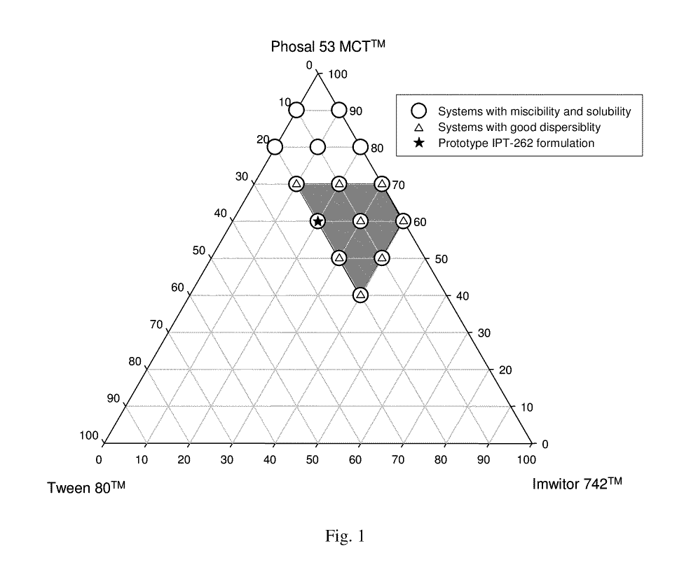

Selection of prototype formulations was based on vehicle miscibility, ABT-263 free base solubility in the vehicle, viscosity of the resulting solution (judged by severity of stringing when released from a dropper) and self-dispersing property of the drug solution (at 10% by weight drug loading), as summarized in Tables 11 and 12 for IPT and IST systems respectively. Schematic phase diagrams for IPT and IST systems (FIGS. 1 and 2) further illustrate the selection process.

As can be seen from Tables 11 and 12 and the phase diagrams in FIGS. 1 and 2, the IPT systems in general provided better vehicle miscibility, drug solubility and dispersibility than the corresponding IST systems. IPT-262 and IST-262 (later replaced by IST-172) were selected as prototype vehicle systems, based on the following rationales.

A phosphatidylcholine-based solvent (for example in the form of Phosal 53 MCT™ or Lipoid S75™ MCT) is needed to ensure both chemical stability (and bioavailability—see below) of the capsule formulation. The amount of such solvent is virtually unlimited due to the low toxicity and high tolerance of lecithin used in oral products.

Polysorbate 80 (especially grades of high purity) is needed to facilitate drug solubility in the vehicle and to enhance self-dispersibility of the lipid formulation. Based on a typical daily dose of ABT-263 (e.g., 200-250 mg) and a maximum daily dose of polysorbate 80 (418 mg), it is reasonable to limit the level of polysorbate 80 to no more than 20% in the vehicle for a prototype formulation with 10% drug loading. Higher levels of polysorbate 80 are also unfavorable due to chemical stability considerations.

In the IPT systems, Imwitor 742™ is needed to reduce the viscosity of the final drug solution to a level that allows for machine capsule filling. In the IST system, Imwitor 742™ is also needed to enhance the miscibility of the vehicle system, since Lipoid S75™ MCT and polysorbate 80 are not miscible at all ratios. However, the amount of Imwitor 742™ is limited to no more than 20% in both prototype systems.

It will be noted from Table 12 that the IST-172 system exhibits poor vehicle miscibility. However, it was found that upon addition of ABT-263 free base the miscibility of the entire system was acceptable; thus the IST-172 formulation became an acceptable prototype liquid for encapsulation.

TABLE 11
Formulation properties of IPT systems containing 10% ABT-263
free base

| | Vehicle | Drug | | |
|---|---|---|---|---|
| Vehicle | miscibility | solubility | Stringing* | Dispersibility (description) |
| IPT-190 | ✓ | ✓ | ++ | Dispersed with vigorous |
| | | | | shaking |
| IPT-280 | ✓ | ✓ | ++ | Dispersed with vigorous |
| | | | | shaking |
| IPT-370 | ✓ | ✓ | ++ | Dispersed with gentle |
| | | | | shaking |
| IPT-460 | ✓ | ✓ | + | Dispersed with gentle |
| | | | | shaking |
| IPT-091 | ✓ | ✓ | +++ | Dispersed with vigorous |
| | | | | shaking |
| IPT-181 | ✓ | ✓ | ++ | Dispersed with vigorous |
| | | | | shaking |
| IPT-271 | ✓ | ✓ | + | Dispersed with vigorous |
| | | | | shaking |
| IPT-361 | ✓ | ✓ | + | Dispersed with vigorous |
| | | | | shaking |
| IPT-451 | ✓ | ✓ | − | Dispersed with gentle |
| | | | | shaking |
| IPT-082 | ✓ | ✓ | +++ | Dispersed with vigorous |
| | | | | shaking |
| IPT-172 | ✓ | ✓ | ++ | Dispersed with gentle |
| | | | | shaking |
| IPT-262 | ✓ | ✓ | + | Dispersed with gentle |
| | | | | shaking |
| IPT-352 | ✓ | ✓ | + | Dispersed with gentle |
| | | | | shaking |
| IPT-442 | ✓ | ✓ | − | Dispersed with gentle |
| | | | | shaking |

✓ vehicle miscible, or drug fully dissolved in vehicle
*stringing: +++ extreme; ++ significant; + slight; − none

TABLE 12
Formulation properties of IST systems containing 10%
ABT-263 free base

| | Vehicle | Drug | | |
|---|---|---|---|---|
| Vehicle | miscibility | solubility | Stringing* | Dispersibility (description) |
| IST-190 | ✓ | ✓ | − | Oil drops spread but did |
| | | | | not disperse until shaken |
| | | | | vigorously |
| IST-280 | ✓ | ✓ | − | Oil drops spread but did |
| | | | | not disperse until shaken |
| | | | | vigorously |
| IST-370 | ✓ | X | n/a | n/a |
| IST-460 | ✓ | X | n/a | n/a |
| IST-091 | X | ✓ | n/a | n/a |
| IST-181 | X | ✓ | − | Dispersed with gentle |
| | | | | shaking |
| IST-271 | ✓ | ✓ | − | Dispersed with gentle |
| | | | | shaking |
| IST-361 | ✓ | X | n/a | n/a |
| IST-451 | ✓ | X | n/a | n/a |
| IST-082 | X | n/a | n/a | n/a |
| IST-172 | X | ✓ | ++ | Rapidly dispersed with |
| | | | | gentle shaking |
| IST-262 | ✓ | ✓ | + | Rapidly dispersed with |
| | | | | gentle shaking |
| IST-352 | ✓ | ✓ | + | Dispersed with gentle |
| | | | | shaking |
| IST-442 | ✓ | X | n/a | n/a |

✓ vehicle miscible, or drug fully dissolved in vehicle
X vehicle immiscible or miscible but turbid, or residual solids present (due to undissolved drug or precipitation)
n/a solution not made due to immiscible vehicle, or dispersibility test not performed due to undissolved drug
*stringing: +++ extreme; ++ significant; + slight; − none

### Example 9

### Antioxidant Selection for Phospholipid-Based Solutions of ABT-263 Free Base

Based on initial antioxidant screening (see Example 6), accelerated stability studies were further conducted on the two prototype formulations using either sodium metabisulfite (NaMTBS) or thioglycerol as an antioxidant, together with 0.01% EDTA.

The solubility of neat NaMTBS in IPT-262 and IST-262 solutions containing 10% ABT-263 free base and 0.01% EDTA (as edetate calcium disodium) was assessed. After 5 days of rotary mixing under ambient temperature conditions, solids remained in all solutions, at NaMTBS solid concentrations as low as 0.05% w/w (or approximately 2% molar concentration relative to ABT-263).

Due to poor lipid solubility of NaMTBS, an alternative way of introducing it to the lipid solution is by adding a concentrated aqueous stock solution of NaMTBS to the lipid solution. For example, a clear solution was obtained when a 50 mg/ml free base solution in Phosal 53 MCT™/ethanol 9:1 v/v was spiked with a 15% w/v NaMTBS solution up to a final NaMTBS concentration of 9.67 mg/ml (or 100% molar concentration relative to ABT-263). However, as the final concentration of NaMTBS was increased to 150% relative molar concentration or higher, using the 15% w/v stock solution, the lipid solution turned turbid. Using a stock solution at a concentration greater than 20% also results in solution turbidity, indicating that both excess amounts of water and NaMTBS can lead to a cloudy solution.

### Example 10

### Sulfoxide Formation in Phospholipid-Based Formulations Containing Antioxidant

Results from a two-week accelerated stability study (stress condition: 40° C., with nitrogen purging), as shown in Table 13, indicated that thioglycerol is not as effective as NaMTBS in inhibiting sulfoxide formation in both prototype formulations.

However, the study results also showed that water added with the NaMTBS can negatively impact chemical stability of the drug solution, and this has been shown to be the case regardless of the ABT-263 form (free base or bis-HCl salt) or the vehicle system used (see Table 14; two-week study at 40° C., with nitrogen purging). For this reason, a final concentration of 0.05% (w/w) NaMTBS was selected, and the concentration of MTBS stock solution should also be kept below about 15% w/v in order to avoid turbidity.

TABLE 13
Sulfoxide formation in ABT-263 prototype liquids for encapsulation

| | | % water | % w/w total sulfoxides | | |
|---|---|---|---|---|---|
| Vehicle | Antioxidant | added* | initial | 1 week | 2 weeks |
| IST-172 | none | 0 | 0.06 | 0.34 | 0.54 |
| IST-172 | 0.05% NaMTBS + | 0.32 | 0.19 | 0.28 | 0.22 |
| | 0.01% EDTA | | | | |
| IST-172 | 0.55% Thioglycerol + | 0 | 0.22 | 0.27 | 0.55 |
| | 0.01% EDTA | | | | |
| IPT-262 | none | 0 | 0.14 | 0.41 | 0.55 |
| IPT-262 | 0.05% NaMTBS + | 0.32 | 0.43 | 0.31 | 0.23 |
| | 0.01% EDTA | | | | |
| IPT-262 | 0.55% Thioglycerol + | 0 | 0.11 | 0.26 | 0.42 |
| | 0.01% EDTA | | | | |

*water as % of formulation contributed by 15% w/v NaMTBS stock solution

TABLE 14
Sulfoxide formation in ABT-263 lipid solutions: effects of NaMTBS and water

| | | ABT-263 | | | | % w/w total |
|---|---|---|---|---|---|---|
| Vehicle | ABT-263 form | concentration | | antioxidant | water % | sulfoxides |
| PE-91 | free base | 50 | mg/ml | none | 0 | 0.47 |
| | (Form I) | | | | | |
| PE-91 | free base | 50 | mg/ml | none | 3.00 | 0.66 |
| | (Form I) | | | | | |
| PE-91 | bis-HCl | 50 | mg/ml | none | 0 | 1.90 |
| | salt | | | | | |
| PE-91 | bis-HCl | 50 | mg/ml | 0.05% NaMTBS + | 0.32 | 0.53 |
| | salt | | | 0.01% EDTA | | |
| PE-91 | bis-HCl | 50 | mg/ml | 0.1% NaMTBS + | 0.61 | 0.84 |
| | salt | | | 0.01% EDTA | | |
| PE-91 | bis-HCl | 50 | mg/ml | 0.2% NaMTBS + | 1.17 | 0.97 |
| | salt | | | 0.01% EDTA | | |
| IST-172 | free base | 100 | mg/g | none | 0 | 0.54 |
| | (Form I) | | | | | |
| IST-172 | free base | 100 | mg/g | 0.05% NaMTBS + | 0.32 | 0.22 |
| | (Form I) | | | 0.01% EDTA | | |
| IST-172 | free base | 100 | mg/g | 0.1% NaMTBS + | 0.61 | 0.22 |
| | (Form I) | | | 0.01% EDTA | | |
| IST-172 | free base | 100 | mg/g | 0.2% NaMTBS + | 1.17 | 0.58 |
| | (Form I) | | | 0.01% EDTA | | |
| IPT-262 | free base | 100 | mg/g | none | 0 | 0.55 |
| | (Form I) | | | | | |
| IPT-262 | free base | 100 | mg/g | 0.05% NaMTBS + | 0.32 | 0.23 |
| | (Form I) | | | 0.01% EDTA | | |
| IPT-262 | free base | 100 | mg/g | 0.1% NaMTBS + | 0.61 | 0.37 |
| | (Form I) | | | 0.01% EDTA | | |
| IPT-262 | free base | 100 | mg/g | 0.2% NaMTBS + | 1.17 | 0.58 |
| | (Form I) | | | 0.01% EDTA | | |

### Example 11

### In Vivo Pharmacokinetics of Prototype Liquid-Filled Capsules

Two 100 mg/g ABT-263 free base liquid-filled capsule prototype formulations were dosed in dogs (single-dose, non-fasting conditions) to evaluate their in vivo pharmacokinetics in comparison with 50 mg/ml oral solutions of ABT-263 free base and bis-HCl salt in Phosal 53 MCT™/ethanol 9:1 v/v with 0.01% EDTA. Formulations tested were:
Formulation 3: 100 mg/g ABT-263 free base in Imwitor 742™/Phosal 53 MCT™/Tween 80™ 20:60:20 (“IPT-262”), liquid-filled capsule;
Formulation 4: 100 mg/g ABT-263 free base in Imwitor 742™/Lipoid S75™ MCT/Tween 80™ 20:60:20 (“IST-262”), liquid-filled capsule;
Formulation 5: 50 mg/ml ABT-263 free base in Phosal 53 MCT™/ethanol 9:1 v/v, oral solution; and
Formulation 6: 50 mg/ml ABT-263 bis-HCl in Phosal 53 MCT™/ethanol 9:1 v/v, oral solution.

Each formulation was evaluated in a group of six dogs at a dose of 50 mg/dog. Formulations 3 (IPT-262) and 4 (IST-262) were dosed in the same group of dogs in a sequential manner, and Formulations 5 and 6 were dosed in a separate group of dogs in a sequential manner. The dogs were fasted overnight prior to dosing, but food was provided 30 minutes prior to dosing. Plasma concentrations of parent drug were determined by HPLC-MS/MS at the completion of each study. Results are presented in Table 15.

TABLE 15
Dog pharmacokinetics of prototype liquid-filled capsules (3 and 4)
versus comparative liquid formulations (5 and 6)

| | Cₘₐₓ | Tₘₐₓ | AUC | |
|---|---|---|---|---|
| Formulation | (μg/ml) | (h) | (μg · h/ml) | F % |
| 3 | 9.8 | 4.7 | 98.6 | 41.9 |
| 4 | 11.0 | 2.5 | 76.8 | 31.8 |
| 5 | 11.3 | 6.0 | 107.8 | 42.5 |
| 6 | 11.9 | 4.5 | 94.1 | 37.7 |

The peak concentration (Cₘₐₓ) of Formulation 3 in plasma was slightly lower than that of Formulation 4, but AUC of Formulation 3 was higher than that of Formulation 4, apparently due to slower absorption. Formulation 4 showed a more consistent but shorter Tₘₐₓ of 2-3 hours after dosing. Liquid-filled capsule Formulation 3 gave comparable plasma Cₘₐₓ, AUC and bioavailability (F %) to that of the oral solutions (Formulations 5 and 6). Based on these results, the IPT-262 prototype (Formulation 3) was selected as a liquid-filled capsule formulation for human clinical studies.

### Example 12

### Storage Stability of Prototype Formulations with and without NaMTBS

Preliminary physical and chemical stability results have been obtained on two laboratory-scale batches of a prototype ABT-263 liquid-filled capsule formulation. The only difference between the two batches is presence or absence of antioxidant (sodium metabisulfite). Composition of the two batches is shown in Table 16.

TABLE 16
Composition of prototype liquid for capsules used in stability study

| | Batch 1 | | Batch 2 | |
|---|---|---|---|---|
| | (with antioxidant) | | (without antioxidant) | |
| | mg per | | mg per | |
| Component | capsule | % w/w | capsule | % w/w |
| ABT-263 free base | 50.0 | 10.0 | 50.0 | 10.0 |
| sodium metabisulfite | 0.25 | 0.05 | — | — |
| edetate calcium disodium | 0.025 | 0.005 | 0.025 | 0.005 |
| water* | 2.48 | 0.50 | 0.23 | 0.05 |
| Phosal 53 MCT ™ | 268.35 | 53.67 | 269.85 | 53.97 |
| mono- and dicaprylic/capric | 89.45 | 17.89 | 89.95 | 17.99 |
| glycerides | | | | |
| polysorbate 80 | 89.45 | 17.89 | 89.95 | 17.99 |
| Total | 500.0 | 100.0 | 500.0 | 100.0 |

*includes water added with sodium metabisulfite and edetate calcium disodium only

The liquids having the composition shown in Table 16 were encapsulated in size 0 hard gelatin capsules and the capsules placed in blister packaging for a chemical stability study. Data after one month storage under various conditions are presented in Table 17. Water content shown in Table 17 is as determined by analysis, and is not directly related to amount of water added with NaMTBS and edetate calcium disodium as in Table 16.

It can be seen from Table 17 that addition of the antioxidant sodium metabisulfite significantly inhibited formation of total sulfoxides, especially under stress storage conditions of 40° C. and 75% RH.

TABLE 17
Chemical stability results for prototype capsules with and without antioxidant

| | | initial | | | 1 month | | |
|---|---|---|---|---|---|---|---|
| | | | | water | | | water |
| | Storage | total | total | content | total | total | content |
| Batch | conditions | sulfoxides | degradants | (%)* | sulfoxides | degradants | (%) |
| 1 (with | 5° C. | n.d. | 0.03% | 2.7 | n.d. | 0.03% | 3.1 |
| antioxidant) | 25° C. | n.d. | 0.03% | 2.7 | n.d. | 0.06% | 3.6 |
| | 60% RH | | | | | | |
| | 40° C. | n.d. | 0.03% | 2.7 | n.d. | 0.03% | 4.8 |
| | 75% RH | | | | | | |
| 2 (without | 5° C. | 0.08% | 0.14% | 3.2 | 0.12% | 0.17% | 3.3 |
| antioxidant) | 25° C. | 0.08% | 0.14% | 3.2 | 0.08% | 0.11% | 3.1 |
| | 60% RH | | | | | | |
| | 40° C. | 0.08% | 0.14% | 3.2 | 0.29% | 0.42% | 3.8 |
| | 75% RH | | | | | | |

*Initial water content of fill solution: 0.4% for batch 1; 0.2% for batch 2
n.d. not detectable

### Example 13

### Preparation of an Illustrative Nanoparticulate Suspension

ABT-263 nanoparticulate suspension formulations were prepared by high-pressure homogenization as described below. The formulations had the following compositions (all percentages expressed as weight/volume) in water:

Formulation 7

| ABT-263 bis-HCl | 5% (4.65% free base equivalent) |
|---|---|
| poloxamer 188 | 3% |

Formulation 8

| | |
|---|---|
| ABT-263 bis-HCl | 5% (4.65% free base equivalent) |
| poloxamer 188 | 3% |
| NaHCO₃ | 8.4% |

Aqueous solutions were prepared containing the indicated amount of poloxamer 188 (Pluronic™ F68) and, in the case of Formulation 8, sodium bicarbonate (NaHCO₃). Crystalline ABT-263 bis-HCl in an amount sufficient to provide a 5% weight/volume (50 mg/ml) suspension was dispersed in each aqueous solution using a Sonifier™ homogenizer (Branson Ultrasonic, Danbury, Conn.). The resulting dispersion was then added to the sample reservoir of a Microfluidizer™ M-110L processor (Microfluidics International Corp., Newton, Mass.) and processed at 12,000 psi (approximately 82.5 MPa) for 2 hours. The sample temperature was maintained throughout at a temperature of 20±2° C. by running the dispersion through a heat exchanger immersed in a water bath connected to a chiller.

The suspensions so obtained (Formulations 7 and 8) were subjected to particle size measurement immediately upon preparation and after storage for 14 days at 5° C. (see Example 14). Formulation 8 was submitted to an oral pharmacokinetic (PK) study in dogs (see Example 15).

### Example 14

### Effect of Sodium Bicarbonate on Particle Size Stability of Nanosuspensions

Formulations 7 and 8 were compared as to their particle size distribution (D₉₀ and D₅₀). Particle size measurement was performed immediately upon preparation of the suspensions (t=0) and after storage for 14 days at 5° C. In addition particle size was measured at t=0 for suspensions following dilution of 1 ml of each suspension in 20 ml 0.9% sodium chloride (NaCl) solution. Data are given in Table 18.

TABLE 18
D₉₀ and D₅₀ particle sizes (μm) of nanosuspension
Formulations 7 and 8

| | |
|---|---|
| Formulation 7 | Formulation 8 |
| (no NaHCO₃) | (8.4% NaHCO₃) |

| | D₉₀ | D₅₀ | D₉₀ | D₅₀ |
|---|---|---|---|---|
| t = 0 | 1.126 | 0.490 | 0.605 | 0.291 |
| 14 d at 5° C. | 1.214 | 0.570 | 0.621 | 0.295 |
| t = 0 in 0.9% NaCl | 1.712 | 0.886 | 0.596 | 0.295 |

### Example 15

### Pharmacokinetics of an Illustrative Nanosuspension

Single-dose pharmacokinetics of Formulation 8 of Example 13 were evaluated in non-fasted beagle dogs (n=4) after a 5 mg/kg oral dose. The formulation was administered in two ways: by oral gavage and in a capsule. Formulation 8 was also administered to histamine-pretreated fasted dogs (n=4), by oral gavage only. For comparative purposes, a solution formulation of ABT-263 bis-HCl in a lipid medium (Formulation C, prepared from ABT-263 bis-HCl powder dissolved to a concentration of 25 mg/ml in a 90:10 mixture of Phosal 53 MCT™ and ethanol) was administered to non-fasted dogs. Formulation C has been used to evaluate ABT-263 in clinical studies.

Serial heparinized blood samples were obtained from a jugular vein of each animal prior to dosing and 0.25, 0.5, 1, 1.5, 2, 3, 4, 6, 9, 12, 15 and 24 hours after administration. Plasma was separated by centrifugation (2,000 rpm for 10 minutes at approximately 4° C.) and ABT-263 was isolated using protein precipitation with acetonitrile.

ABT-263 and an internal standard were separated from each other and from co-extracted contaminants on a 50×3 mm Keystone Betasil CN™ 5 μm column with an acetonitrile/0.1% trifluoroacetic acid mobile phase (50:50 by volume) at a flow rate of 0.7 ml/min. Analysis was performed on a Sciex API3000™ biomolecular mass analyzer with a heated nebulizer interface. ABT-263 and internal standard peak areas were determined using Sciex MacQuan™ software. The plasma drug concentration of each sample was calculated by least squares linear regression analysis (non-weighted) of the peak area ratio (parent/internal standard) of the spiked plasma standards versus concentration. The plasma concentration data were submitted to multi-exponential curve fitting using WinNonlin 3 (Pharsight).

The area under the plasma concentration-time curve from 0 to t hours (time of the last measured plasma concentration, which here is 24 hours) after dosing (AUC₀₋₂₄) was calculated using the linear trapezoidal rule for the plasma concentration-time profiles.

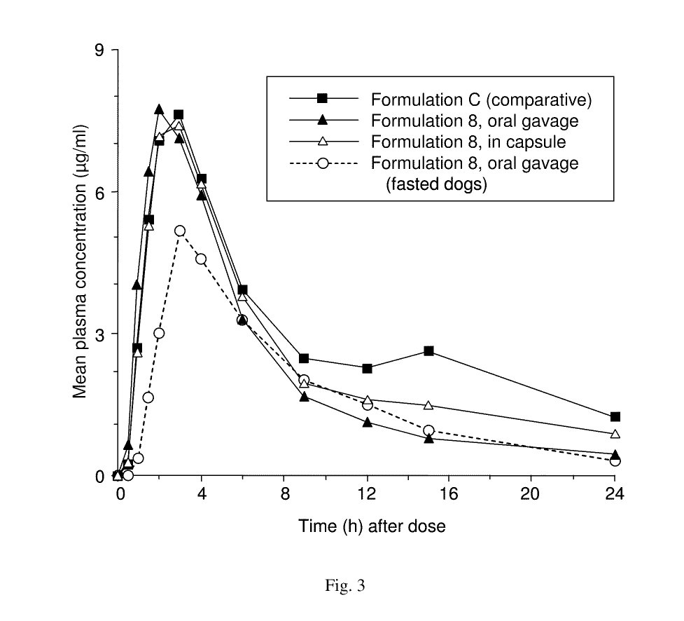

Mean plasma concentrations over 24 hours after dosing are shown in FIG. 3.

Calculated mean PK parameters are summarized in Table 19.

TABLE 19
PK parameters (mean ± SEM) in dogs (non-fasted unless
otherwise indicated)

| | Cₘₐₓ | Tₘₐₓ | AUC₀₋₂₄ | Bioavailability |
|---|---|---|---|---|
| | (μg/ml) | (h) | (μg · h/ml) | F % |
| Formulation C | 9.09 ± 1.33 | 6.3 ± 1.6 | 54.5 ± 6.3 | 22.4 ± 2.6 |
| (comparative) | | | | |
| Formulation 8, | 7.78 ± 0.35 | 2.3 ± 0.3 | 45.2 ± 2.6 | 19.9 ± 1.2 |
| oral gavage | | | | |
| Formulation 8, | 7.52 ± 2.46 | 3.0 ± 0.4 | 48.3 ± 12.4 | 21.3 ± 5.5 |
| in capsule | | | | |
| Formulation 8, | 5.56 ± 0.46 | 3.3 ± 0.3 | 35.6 ± 0.6 | 15.7 ± 0.2 |
| oral gavage | | | | |
| (fasted dogs) | | | | |

### Example 16

### Preparation of Solid Dispersions of ABT-263 bis-Hcl

ABT-263 bis-HCl crystalline salt was mixed with a surfactant and a water-soluble polymer in the following weight ratios:

10.8% ABT-263 salt (10% free base equivalent); 10% surfactant; 79.2% polymer

21.5% ABT-263 salt (20% free base equivalent); 10% surfactant; 68.5% polymer

32.3% ABT-263 salt (30% free base equivalent); 10% surfactant; 57.7% polymer

43% ABT-263 salt (40% free base equivalent); 10% surfactant; 47% polymer

The surfactant in different series was TPGS, Span™ 20 or Tween™ 20. The polymer in different series was copovidone (Kollidon™ VA 64), povidone K-30 or HPMC-AS.

The mixture of ingredients in each case was dissolved in methanol. The methanol was removed at 65° C. in vacuo using a Genevac™ system, and the resulting solid dispersion was allowed to cool to ambient temperature.

The solid dispersion in each case was sieved through a 40-mesh screen to provide a powder of reduced particle size. The resulting powders were used for determination of T_{g} by differential scanning calorimetry (DSC), residual solvent and moisture determination by thermogravimetric analysis (TGA), characterization of crystallinity or lack thereof by powder X-ray diffraction (PXRD), and determination of physical stability when stored at 25° C./60% RH and at 40° C./75% RH.

The solid dispersion powder in each case was blended with ProSolv HD 90™, croscarmellose sodium and sodium stearyl fumarate at a weight ratio of 82:15:2:1. The resulting blend was filled into hard gelatin capsules of a size, depending on drug loading, to provide a 50 mg unit dose of ABT-263. The capsules were tested for dissolution in a pH 6.5 buffer medium containing 7.6 mM Tween™ 80, using USP apparatus II (see Example 17 below).

All tested solid dispersions of ABT-263 bis-HCl prepared as above were found to have a T_{g} in the range of 70-110° C. TGA showed that the copovidone/HPMC-AS dispersions had the lowest moisture content (2-4%) and the povidone dispersions, regardless of surfactant used, had the highest moisture content (8-10%). PXRD showed no crystallinity, i.e., the ABT-263 bis-HCl was amorphous in all solid dispersions. Only the ABT-263 bis-HCl solid dispersions prepared with HPMC-AS as the polymeric carrier showed acceptable storage stability for one month. Where povidone or copovidone was used, a tendency for deliquescence was observed in open-dish storage stability testing at both at 25° C./60% RH and at 40° C./75% RH.

### Example 17

### Preparation of Solid Dispersions of ABT-263 Free Base

ABT-263 bis-HCl crystalline salt was dissolved in acetone, and NaOH was added to convert the ABT-263 bis-HCl to free base. The NaCl by-product precipitated and was removed by filtration.

To the resulting ABT-263 free base solution in acetone were added a surfactant and a water-soluble polymer in the following weight ratios:

10% ABT-263 free base; 10% surfactant; 80% polymer

20% ABT-263 free base; 10% surfactant; 70% polymer

30% ABT-263 free base; 10% surfactant; 60% polymer

40% ABT-263 free base; 10% surfactant; 50% polymer

The surfactant in different series was TPGS, Span™ 20 or Tween™ 20. The polymer in different series was copovidone (Kollidon™ VA 64) or HPMC-AS.

The acetone was removed at 65° C. in vacuo using a Genevac™ system, and the resulting solid dispersion was allowed to cool to ambient temperature.

The solid dispersion in each case was sieved through a 40-mesh screen to provide a powder of reduced particle size. The resulting powders, as in Example 16, were used for determination of T_{g} by DSC, residual solvent and moisture determination by TGA, characterization of crystallinity or lack thereof by PXRD, and determination of physical stability when stored at 25° C./60% RH and at 40° C./75% RH.

The solid dispersion powder in each case was blended with ProSolv HD 90™, croscarmellose sodium and sodium stearyl fumarate at a weight ratio of 82:15:2:1. The resulting blend was filled into hard gelatin capsules of a size, depending on drug loading, to provide a 50 mg unit dose of ABT-263. The capsules were tested for dissolution in a pH 6.5 buffer medium containing 7.6 mM Tween™ 80 (see Example 18 below).

All tested solid dispersions of ABT-263 free base prepared as above were found to have a T_{g} in the range of 70-110° C. TGA showed that the copovidone and HPMC-AS dispersions had low moisture content (2-4%). PXRD showed no crystallinity, i.e., the ABT-263 free base was amorphous in all solid dispersions. The ABT-263 free base solid dispersions prepared with copovidone or HPMC-AS as the polymeric carrier showed acceptable storage stability for one month without any sign of deliquescence.

### Example 18

### Dissolution Profiles of Solid Dispersions

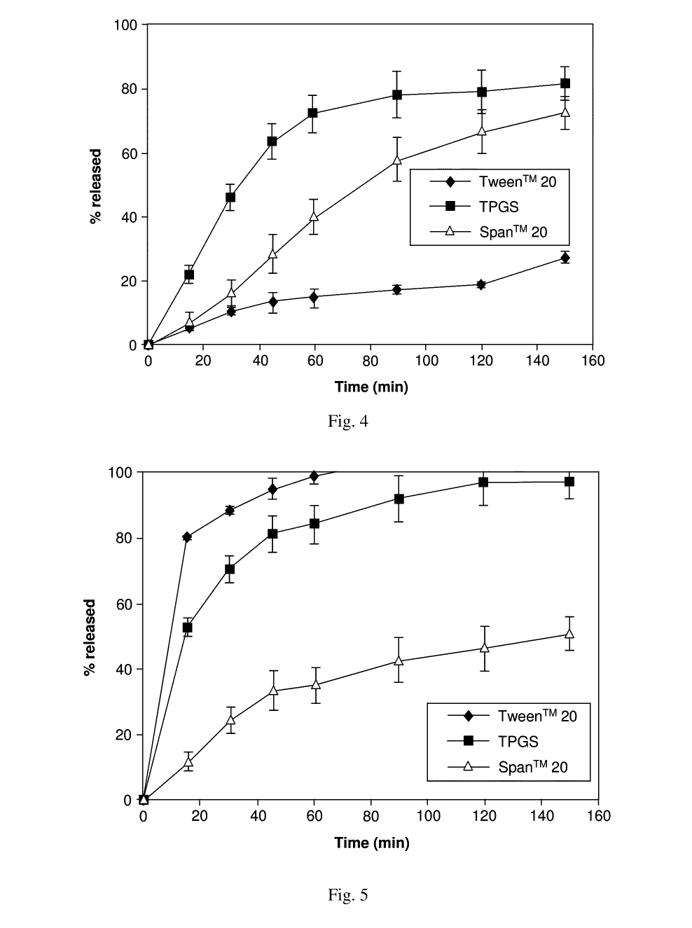

Representative dissolution (drug release) profiles in a pH 6.5 buffered medium containing 7.6 mM Tween™ 80 are shown in FIG. 4 (ABT-263 bis-HCl) and FIG. 5 (ABT-263 free base).

As shown in FIG. 4, at a 20% drug-loading level, the ABT-263 bis-HCl solid dispersions with 68.5% copovidone and 10% TPGS showed a moderate rate of drug release that plateaued at about 80% release. Release from similar dispersions having Span™ 20 or, especially, Tween™ 20 as the surfactant was much slower.

By contrast, as shown in FIG. 5, at the same 20% drug-loading level, the ABT-263 free base solid dispersions with 70% copovidone and 10% of either Tween™ 20 or TPGS showed rapid dug release. Only the Span™ 20 surfactant resulted in much slower release in the case of the free base dispersion.

Release rate was drug-loading-dependent in both ABT-263 bis-HCl and free base dispersion formulations, the 20% dispersions showing faster release than the 30% or 40% dispersions in both cases.

Unlike the analogous solid dispersion prepared from the ABT-263 free base, the solid dispersion containing ABT-263 bis-HCl, copovidone and Tween™ 20 showed shell formation. This shell formation is believed to be caused by precipitation of the drug on the surface of the capsule fill plug.

In a separate study, solid dispersions of ABT-263 bis-HCl in a copovidone matrix with and without replacement of 5% copovidone with HPMC-AS showed slower drug release in presence of HPMC-AS.

### Example 19

### Effect of Polymeric Carrier on Dissolution Profile of ABT-263 bis-HCl Dispersions

Solid dispersions with different polymeric carriers were tested to observe impact of the polymeric carriers on dissolution rates. Four solid dispersions were prepared with ABT-263 bis-HCl salt (20% free base equivalent), 10% TPGS and the following polymeric carriers:

povidone only

50% povidone+50% copovidone

25% povidone+75% copovidone

copovidone only

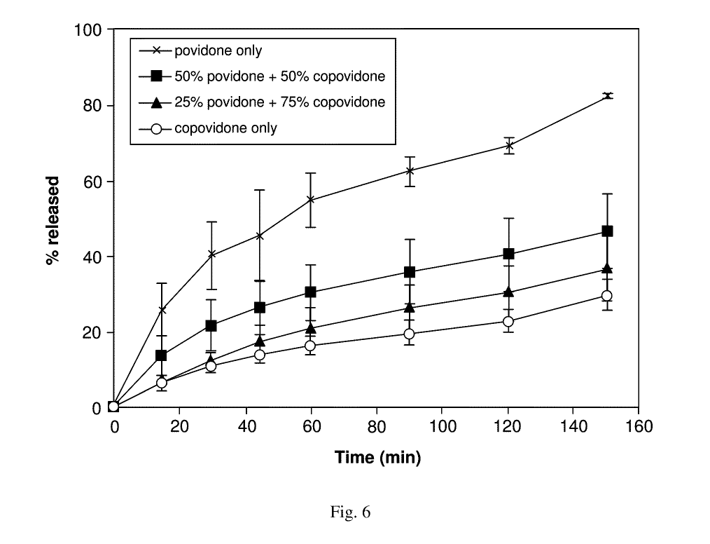

Dissolution profiles of the four solid dispersions are shown in FIG. 6. Drug release rate increased with increasing levels of povidone.

### Example 20

### Pharmacokinetics of ABT-263 bis-HCl Dispersions in a Dog Model

Single-dose pharmacokinetics of two ABT-263 solid dispersions were evaluated in non-fasted beagle dogs (n=6) after a 50 mg/kg oral dose followed by 10 ml water. Serial heparinized blood samples were obtained from a jugular vein of each animal prior to dosing and 0.25, 0.5, 1, 1.5, 2, 3, 4, 6, 9, 12, 15 and 24 hours after administration. Plasma was separated by centrifugation (2,000 rpm for 10 minutes at approximately 4° C.) and ABT-263 was isolated using protein precipitation with acetonitrile.

Two ABT-263 bis-HCl solid dispersions (those of Example 19 containing povidone only or copovidone only) were compared. The powdered dispersions were blended with ProSolv HD 90™, croscarmellose sodium and sodium stearyl fumarate in an 82:15:2:1 weight ratio and the blend filled into capsules.

ABT-263 and an internal standard were separated from each other and from co-extracted contaminants on a 50×3 mm Keystone Betasil CN™ 5 μm column with an acetonitrile/0.1% trifluoroacetic acid mobile phase (50:50 by volume) at a flow rate of 0.7 ml/min. Analysis was performed on a Sciex API3000™ biomolecular mass analyzer with a heated nebulizer interface. ABT-263 and internal standard peak areas were determined using Sciex MacQuan™ software. The plasma drug concentration of each sample was calculated by least squares linear regression analysis (non-weighted) of the peak area ratio (parent/internal standard) of the spiked plasma standards versus concentration. The plasma concentration data were submitted to multi-exponential curve fitting using WinNonlin 3 (Pharsight).

The area under the plasma concentration-time curve from 0 to t hours (time of the last measured plasma concentration) after dosing (AUC₀₋ₜ) was calculated using the linear trapezoidal rule for the plasma concentration-time profiles. The residual area extrapolated to infinity, determined as the final measured plasma concentration (Cₜ) divided by the terminal elimination rate constant (β), was added to AUC₀₋ₜ to produce the total area under the curve (AUC_{0-∞}). The bioavailability was calculated as the dose-normalized AUC_{0-∞}from oral dosing divided by the corresponding value derived from i.v. (intravenous) dosing, administered as a slow bolus to a jugular vein under light ether anesthetic.

PK parameters for the povidone-only and copovidone-only dispersions are presented in Table 20.

TABLE 20
PK parameters of solid dispersion compositions in dog (n = 6)

| | | Cₘₐₓ/D | | | AUC/D | |
|---|---|---|---|---|---|---|
| | Cₘₐₓ | μg/ml per | | AUC | μg · h/ml | |
| Composition | μg/ml | mg/kg | Tₘₐₓ h | μg · h/ml | per mg/kg | F % |
| povidone | 5.6 | 1.16 | 9.8 | 39.3 | 7.9 | 16.4 |
| copovidone | 9.6 | 1.78 | 4.5 | 64.9 | 11.9 | 24.7 |

Although the ABT-263 bis-HCl dispersion prepared with povidone was shown in Example 19 to provide a better release rate than copovidone, it had poorer bioavailability in this dog study than a comparable dispersion prepared with copovidone.

### Example 21

### Pharmacokinetics of Illustrative Solid Dispersions in a Dog Model

Single-dose pharmacokinetics of two ABT-263 solid dispersions were evaluated in non-fasted beagle dogs (n=6), following the same protocol as that of Example 20. Two ABT-263 solid dispersions (Dispersions I and II) were prepared. Dispersion I, prepared substantially according to the process of Example 17, contained 10% ABT-263 free base, 10% TPGS and 80% copovidone. The powdered dispersion was filled into capsules without any additional ingredients to prepare Formulation 9. Dispersion II, prepared substantially according to the process of Example 16, contained 13.11% ABT-263 bis-HCl (12.18% free base equivalent), 15% TPGS and 71.89% povidone. The powdered dispersion was blended with ProSolv HD 90™, sodium starch glycolate and sodium stearyl fumarate in an 82:15:2:1 weight ratio and the blend filled into capsules to prepare Formulation 10.

PK parameters for Formulations 9 and 10 are presented in Table 21.

TABLE 21
PK parameters of solid dispersion compositions in dog (n = 6)

| | | Cₘₐₓ/D | | | AUC/D | |
|---|---|---|---|---|---|---|
| | Cₘₐₓ | μg/ml per | | AUC | μg · h/ml | |
| Formulation | μg/ml | mg/kg | Tₘₐₓ h | μg · h/ml | per mg/kg | F % |
| 9 | 7.5 | 1.50 | 8.5 | 59.0 | 11.2 | 24.6 |
| 10 | 6.4 | 1.24 | 7.8 | 39.2 | 7.4 | 16.3 |

The ABT-263 bis-HCl dispersion (Formulation 10) prepared with povidone had poorer bioavailability in this dog study than the ABT-263 free base dispersion (Formulation 9) prepared with copovidone.

### Example 22

### Preparation and Characterization of Solid Dispersion Products

Formulations of various compositions were produced as shown in Table 22 below. ABT-263 was mixed in a blender with a pre-granulated mixture of Copovidone (copolymer of N-vinyl pyrrolidone and vinyl acetate) and the solubilizer(s). Where indicated, 1% of colloidal silicon dioxide was added to improve flow properties. The powdery mixture was extruded in a Leistritz micro 18 GMP-extruder at an extrusion temperature as shown in Table 22.

Absolute bioavailability compares the bioavailability (estimated as the area under the curve, or AUC) of the active drug in systemic circulation following oral administration with the bioavailability of the same drug following intravenous administration. In Table 22 the bioavailability (F %) was determined after administering an ABT-263 dose of 50 mg to fed dogs.

TABLE 22
Composition, stability and biovailability in dogs of solid dispersions

| | Formulation | | | | | | | |
|---|---|---|---|---|---|---|---|---|
| | 11 | 12 | 13 | 14 | 15 | 16 | 17 | 18 |
| ABT-263 bis-HCl (%) | 10 | 10.7 | 10.7 | 10.7 | 10 | 10 | 10 | 10 |
| copovidone (%) | 80 | 72.3 | 72.3 | 72.3 | 80 | 79 | 80 | 79 |
| polysorbate 20 (%) | 10 | 10 | | 5 | | | | |
| Span ™ 20 (%) | | | | | 5 | 5 | | |
| Vitamin E-TPGS ™ (%) | | | 10 | 2 | | | 5 | 5 |
| sodium lauryl sulfate (%) | | 6 | 6 | 6 | | | | |
| propylene glycol (%) | | | | 3 | 5 | 5 | 5 | 5 |
| colloidal silicon dioxide (%) | | 1 | 1 | 1 | | 1 | | 1 |
| extrusion temperature (° C.) | 140 | 140 | 140 | 140 | 140 | 140 | 130 | 130 |
| sum of degradation products (%) | 1.83 | 1.11 | 1.22 | 1.05 | 2.78 | 1.07 | 1.68 | 0.93 |
| sum of sulfoxides (%) | n.d. | 0.77 | 0.71 | 0.69 | n.d. | n.d. | n.d. | n.d. |
| bioavailability (F %) | 27.5 | 32.6 | 25.9 | 27.0 | 31.7 | n.d. | 26.7 | n.d. |
| F % for Formulation C | 22.4 | 31.5 | 29.2 | 29.2 | 22.4 | n.d. | 22.4 | n.d. |
| in same study | | | | | | | | |
| relative F %** | 122.8 | 103.5 | 88.7 | 92.5 | 141.5 | n.d. | 119.2 | n.d. |

| | Formulation | | | | | | |
|---|---|---|---|---|---|---|---|
| | 19 | 20 | 21 | 22 | 23 | 24 | 25 |
| ABT-263 form and amount (%) | bis- | bis- | bis- | Na | free | free | bis- |
| | HCl | HCl | HCl | salt | base | base | HCl |
| | 10.7 | 10.7 | 10.7 | 10 | 10 | 10 | 10.7 |
| copovidone (%) | 78.3 | 78.3 | 72.3 | 79 | 79 | 79 | 72.3 |
| polysorbate 20 (%) | | | | 10 | | | |
| Span ™ 20 (%) | | | | | | | 10 |
| Vitamin E-TPGS ™ (%) | 5 | 5 | 5 | | 10 | 10 | |
| sodium lauryl sulfate (%) | | | 5 | | | | 6 |
| propylene glycol (%) | 5 | 5 | 5 | | | | |
| colloidal silicon dioxide (%) | 1 | 1 | 1 | 1 | 1 | 1 | 1 |
| extrusion temperature (° C.) | 130 | 135 | 140 | 130 | 125 | 130 | 130 |
| sum of degradation products (%) | 0.66 | 0.83 | 1.23 | 0.73 | 0.80 | 0.41 | 1.27 |
| sum of sulfoxides (%) | 0.37 | 0.42 | 0.72 | 0.29 | 0.43 | 0.30 | 0.62 |
| bioavailability (F %) | n.d. | 29.6 | n.d. | 32.1 | 33.7 | n.d. | n.d. |
| F % for Formulation C in same study | n.d. | n.d. | n.d. | 31.5 | n.d. | n.d. | n.d. |
| relative F %** | n.d. | n.d. | n.d. | 101.9 | n.d. | n.d. | n.d. |

n.d. not determined
**calculated by taking bioavailability (F %) for Formulation C as 100%

### Example 23

### Bioavailability Evaluation of Solid Dispersions

### (a) Protocol for Oral Bioavailability Studies

For bioavailability evaluation, extrudates as described in Example 22 were milled and filled into capsules. Each capsule contained 50 mg ABT-263.

The dose response and food effect for two formulations were evaluated in beagle dogs (both genders, approximate weight: 10 kg). Groups of 5 dogs each received a 50 mg (1 capsule/dog), 100 mg (2 capsules/dog) or 200 mg (4 capsules/dog) oral dose of ABT-263 under both fasting and fed conditions. The dose was followed by approximately 10 ml water. For all studies, beagle dogs were fasted overnight prior to dosing, but were permitted water ad libitum. Food was returned to the dogs approximately 30 minutes prior to dosing (fed conditions) or 4 hours after dosing (fasting conditions). A washout/recovery period of one week separated the two dosing periods. Blood samples were obtained from each animal prior to dosing and at convenient time points chosen among 0.25, 0.5, 1.0, 1.5, 2, 3, 4, 6, 9, 12, 15, 24, 36 and 48 hours after drug administration. The plasma was separated from the red cells by centrifugation and frozen at −30° C. until analysis. Concentrations of ABT-263 were determined by reverse phase HPLC-MS/MS following liquid-liquid extraction of the plasma samples. The area under the curve (AUC) was calculated by the trapezoidal method over the time course of the study. Each dosage form was evaluated in a group containing 5 dogs; the values reported are averages for each group of dogs.

### (b) Influence of Dosage and Application to Fasted or Fed Dogs

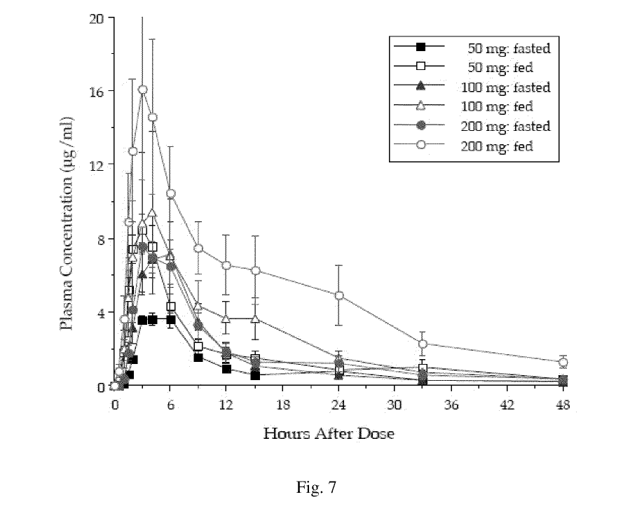

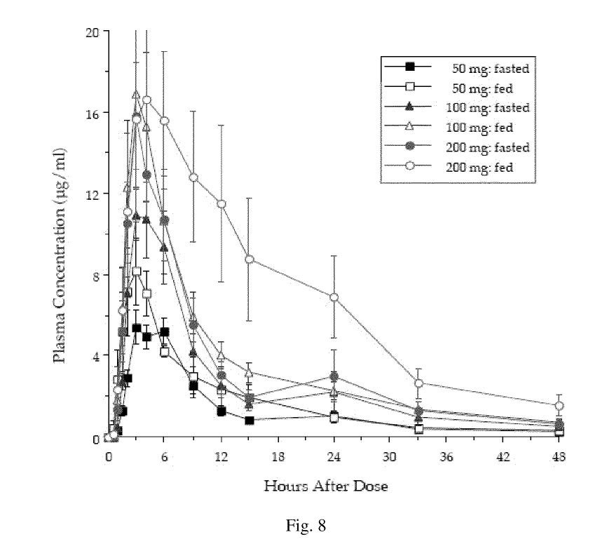

Formulations 16 or 18 of ABT-263 as defined in Table 22 were administered to fasted or fed dogs in dosages corresponding to the amounts of ABT-263 as indicated in FIG. 7 and FIG. 8. Subsequently, the plasma concentrations of ABT-263 were determined from blood samples taken at the indicated time points. In FIG. 7 and FIG. 8, open and closed symbols represent fed or fasted dogs, respectively. Squares, triangles and circles represent a dose of 50 mg, 100 mg or 200 mg ABT-263, respectively.

For both formulations plasma concentrations of ABT-263 were higher when administered to fed dogs. This effect was more prominent at higher dosages of 100 mg and 200 mg. In fed dogs a dose linearity could be observed. AUC values of Formulation 16 in fasted dogs were 40-60% lower than in fed dogs. When Formulation 18 was administered AUC values were approximately 30% lower in fasted dogs.

(c) Comparison of a Free Base Formulation Vs. a bis-HCl Salt Formulation

Fed dogs received orally one of the following two formulations as one capsule containing Formulation 23 or Formulation 20 as indicated in Table 22, equivalent to an amount of 50 mg ABT-263.

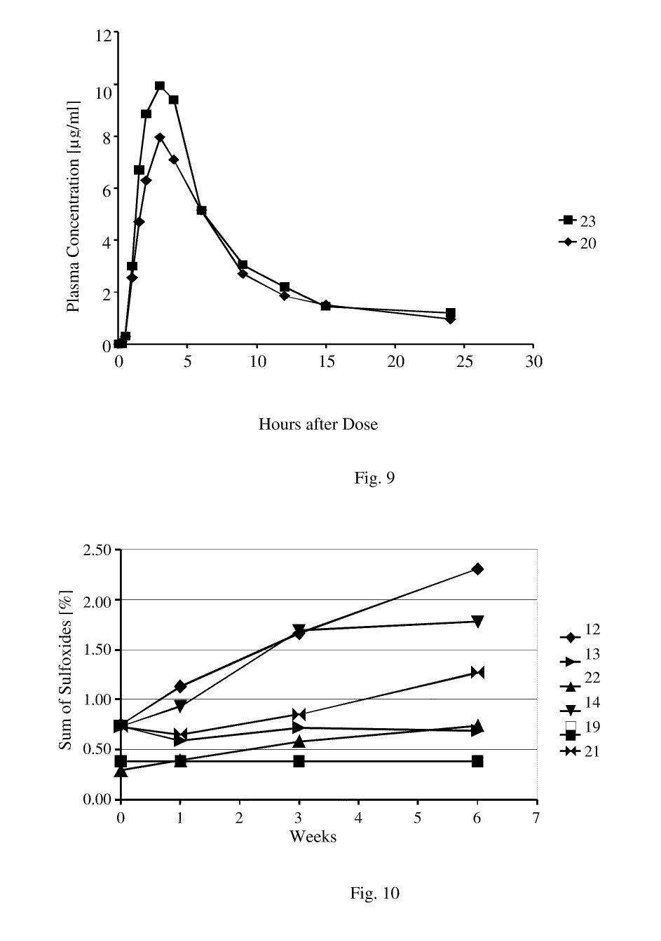

The plasma concentrations of ABT-263 were determined from blood samples taken at the time points as indicated in FIG. 9, which shows the mean plasma concentration of five dogs treated with Formulation 23 or Formulation 20, respectively.

The bioavailability data obtained from this experiment are summarized in Table 23 below (shown as mean value of 6 animals; standard deviation in brackets).

TABLE 23
Pharmacokinetics in fed dogs of solid dispersion formulations

| Formulation | Cₘₐₓ | Cₘₐₓ/D | Tₘₐₓ | AUC | AUC/D | F % |
|---|---|---|---|---|---|---|
| 23 (free base) | 10.4 (2.1) | 2.03 | 3.2 (0.5) | 78.7 (15.7) | 15.3 | 33.7 (5.5) |
| 20 (bis-HCl salt) | 8.6 (0.7) | 1.74 | 3.6 (0.6) | 67.6 (7.9) | 13.4 | 29.6 (3.4) |

Cₘₐₓ maximum concentration of ABT-263 in plasma (μg/ml)
Cₘₐₓ/D maximum concentration per dose (μg/ml per mg/kg)
Tₘₐₓ time to maximum plasma concentration (h)
AUC area under the plasma concentration curve (μg · hr/ml)
AUC/D area under curve per dose (μg · hr/ml per mg/kg)
F % average bioavailability

### Example 24

### Storage Stability

For selected formulations (Formulations 16 and 18 according to Table 22) the storage stability was determined. The formulations were kept in closed containers at ambient conditions (approximately 19° C. to 25° C. at RH of 60% or less). The ABT-263 content and the content of degradation products of the active ingredient including sulfoxides were determined at the beginning of the storage period (initial value) and after 4 months by separation via HPLC (or HPLC) and detection with a UV/VIS detector. The results are shown in Table 24 below.

TABLE 24
Storage stability of solid dispersion formulations

Formulation

| | 16 | 18 |
|---|---|---|
| ABT-263 content (initial) | 97.8% | 97.0% |
| degradation products (initial) | 1.07% | 0.93% |
| ABT-263 content (after 4 months) | 96.7% | 98.9% |
| degradation products (after 4 months) | 1.16% | 0.96% |

The formulations were chemically stable as content and impurity levels remained unchanged upon storage.

### Example 25

### Determination of Sulfoxide Formation

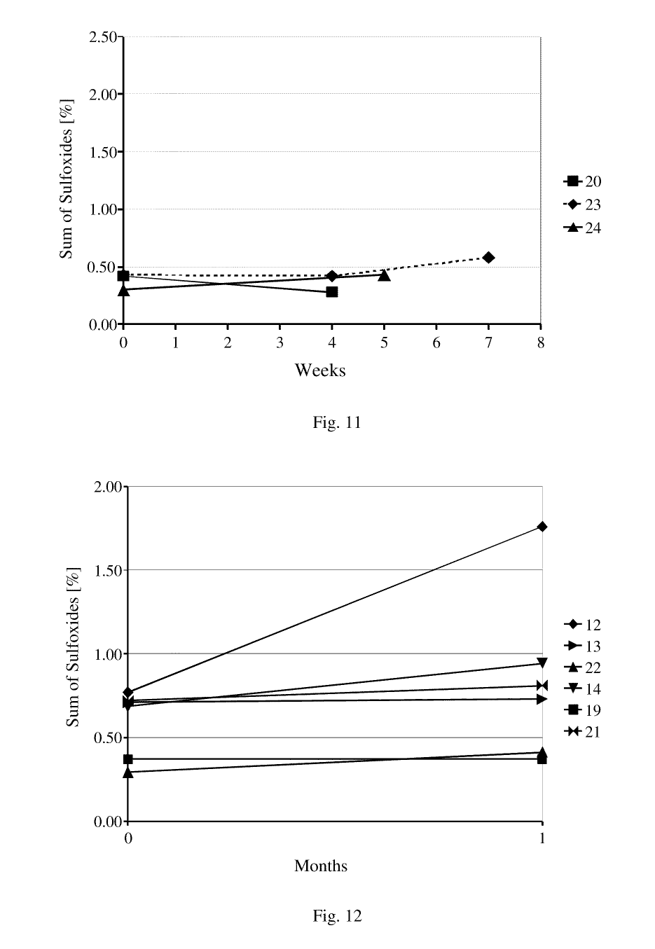

Formulations 12, 13, 22, 14, 19, 21, 20, 23 and 24 as defined in Table 22 were assessed for sulfoxide formation in an accelerated stability study, using exposure in an open dish at a relative humidity of 40° C./75%. Sulfoxide content was determined at the beginning of the experiment (less than 0.8% in all cases), after 1 week, 3 weeks and 6 weeks for the formulations referred to in FIG. 10, and at time points chosen among 4 weeks, 5 weeks and 7 weeks for the formulations referred to in FIG. 11.

The data shown in FIG. 10 indicate that lower extrusion temperatures cause lower contents of sulfoxides. Comparatively low levels of sulfoxides were also observed in the formulations referred to in FIG. 11, all of which were extruded at temperatures of 135° C. or less. Sulfoxide contents increased most significantly with Formulations 12 and 14, both of which contain polysorbate 20. Therefore, the inclusion of polysorbate 20 appears to promote formation of sulfoxides.

In a second experiment sulfoxide formation was determined in samples which were kept in closed 1.5 oz HDPE bottles at a temperature and relative humidity of 40° C./75%. The results are shown in FIG. 12 and FIG. 13.

### Example 26

### Crystallinity of ABT-263 Extrudates

Formulations 19, 12, 23 and 24 as defined in Table 22 were manufactured, using the process parameters as indicated in Table 25 below. The extrudates were evaluated for the presence of crystalline active ingredient by polarization microscopy.

TABLE 25
Crystallinity of ABT-263 extrudates

| Formulation | | | |
|---|---|---|---|
| 20 | 23 | 24 | 25 |

Process parameters:

| | | | | | | | | |
|---|---|---|---|---|---|---|---|---|
| feed rate | 0.5 | kg/h | 1.0 | kg/h | 1.0 | kg/h | 0.5 | kg/h |
| temperature | 135° | C. | 125° | C. | 130° | C. | 130° | C. |

Process data:

| | | | | |
|---|---|---|---|---|
| crystallinity | detected | not detected | not detected | not detected |

### Example 27

### Crystallinity of ABT-263 Extrudates Upon Prolonged Storage

Various extrudates as indicated in Table 26 were kept at accelerated aging conditions in open dishes or closed bottles. At the indicated time points the presence of crystalline active ingredient was evaluated by polarization microscopy.

TABLE 26
Physical stability (crystallinity) of ABT-263 extrudates

| | Time | | | | |
|---|---|---|---|---|---|
| | 0 weeks | 1 week | 3 weeks | 6 weeks | 1 month |
| Storage | | open dish 40° C./75% RH | | | 1.5 oz HDPE |
| conditions | | | | | bottles, closed, |
| | | | | | 40° C./75% RH |
| 12 | detected (++) | detected (++) | detected (++) | detected (++) | detected (++) |
| 13 | detected (++) | detected (++) | detected (++) | detected (++) | detected (++) |
| 22 | not detected | not detected | not detected | not detected | not detected |
| 14 | not detected | detected (+) | detected (++) | detected (++) | detected (++) |
| 19 | detected (+) | not detected | not detected | detected (+) | detected (+) |
| 21 | detected (+) | detected (++) | detected (++) | detected (++) | detected (++) |
| 20 | detected (+) | detected (+) | detected (+) | detected (+) | detected (+) |
| 23 | not detected | not detected | not detected | not detected | not detected |
| 24 | not detected | not detected | not detected | not detected | not detected |

(+) few crystals detected
(++) numerous crystals detected

### Example 28

### Manufacture of Tablets

Following the procedure of Example 22, an extrudate was obtained from the solid dispersion product ingredients listed in Table 27 below. Extrudates from Example 22 were milled and the powder was blended with the tableting excipients listed in Table 27. A single-punch tablet press was used to prepare tablets containing 50 mg ABT-263.

TABLE 27
Tablet composition

| | Formulation | | |
|---|---|---|---|
| | 26 | 27 | 28 |
| extrudate (ABT-263 free | 98% | 83% | 83% |
| base:copovidone:Vitamin | | | |
| E-TPGS ™:colloidal | | | |
| silicon dioxide 10:79:10:1) | | | |
| croscarmellose sodium | | | 15% |
| mannitol | | 15% | |
| colloidal silicon dioxide | 1.0% | 1.0% | 1.0% |
| sodium stearyl fumarate | 1.0% | 1.0% | 1.0% |
| Total tablet mass | 510.2 mg | 602.4 mg | 602.4 mg |

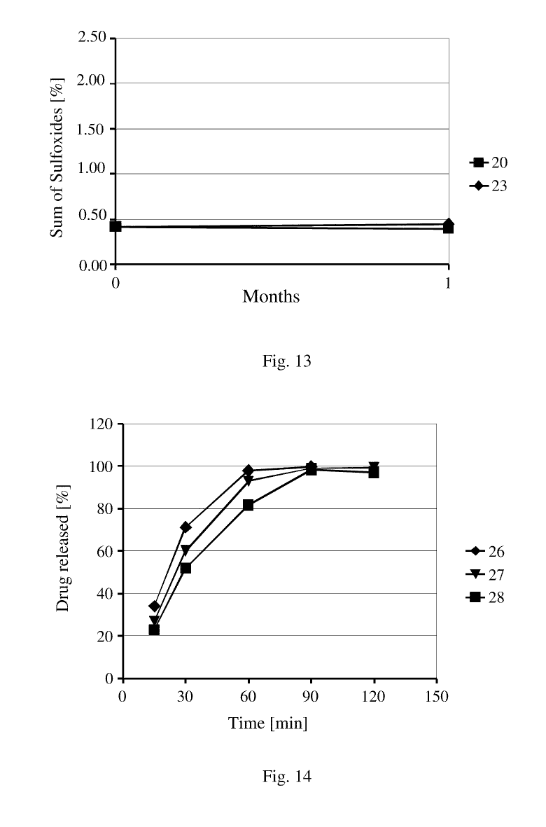

The tablets were immersed in 0.1N HCl at a temperature of 37° C. (to mimic stomach conditions) and stirred by paddle rotation at a speed of 75 rpm. The amount of released ABT-263 was determined at various time points by HPLC-UV/VIS. The results are shown in FIG. 14.

### Example 29

### PK Studies of ABT-263 Solid Tablets in Dogs

PK studies were performed in non-fasting beagle dogs (n=3) at a single dose of 50 mg ABT-263 free base equivalent. Plasma concentrations of the drug were determined by high pressure liquid chromatography mass spectrometry (HPLC-MS) and PK parameters were calculated by standard procedures in the art.

Eleven tablet compositions of the invention (Formulations 26-36) were tested. API (ABT-263 bis-HCl in all cases) was unmilled unless otherwise indicated. Composition of each of Formulations 26-30 is as shown in Table 28.

TABLE 28
Composition of tablets (Formulations 26-30)

| | Amount (% by weight) | | | | |
|---|---|---|---|---|---|
| Ingredient | 26 | 27 | 28 | 29 | 30 |
| ABT-263 bis-HCl | 10.00 | 10.00 | 10.00 | 10.75 | 10.75 |
| Avicel 101 ™ | 81.25 | 84.25 | 50.75 | 30.00 | 30.00 |
| mannitol | | | 20.00 | 40.00 | 40.00 |
| povidone K-30 | 3.00 | 3.00 | 5.00 | 5.00 | 3.00 |
| crospovidone | 1.50 | 1.50 | | | |
| poloxamer (Pluronic ™ F127) | 4.00 | 1.00 | 4.00 | | |
| TPGS | | | | 4.00 | 6.00 |
| sodium starch glycolate | | | 10.00 | 10.00 | 10.00 |
| magnesium stearate | 0.25 | 0.25 | 0.25 | 0.25 | 0.25 |

Formulations 31-36 comprised intra- and extragranular components. Composition of each of these formulations is as shown in Table 29.

TABLE 29
Composition of tablets (Formulations 31-36)

| | Amount (% by weight) | | | | | |
|---|---|---|---|---|---|---|
| Ingredient | 31 | 32 | 33 | 34 | 35 | 36 |

Intragranular

| | | | | | | |
|---|---|---|---|---|---|---|
| ABT-263 bis-HCl | 10.75 | 10.75 | 10.75 | 21.50 | 10.75 | 21.50 |
| Avicel 101 ™ | 33.00 | 34.00 | 30.00 | 29.25 | 30.00 | 29.25 |
| mannitol | 20.00 | 20.00 | 20.00 | 20.00 | 30.00 | 20.00 |
| PVP 30 | 5.00 | 5.00 | 5.00 | 5.00 | 5.00 | 5.00 |
| poloxamer (Pluronic ™ F127) | 1.00 | | | | | |
| sodium starch glycolate | | | 5.00 | 5.00 | 5.00 | 5.00 |
| Cremophor EL ™ | | | 4.00 | 4.00 | | |
| TPGS | | | | | 4.00 | 4.00 |

Extragranular

| | | | | | | |
|---|---|---|---|---|---|---|
| Avicel 101 ™ | 20.00 | 20.00 | 20.00 | 10.00 | 20.00 | 20.00 |
| sodium starch glycolate | 5.00 | 5.00 | 5.00 | 5.00 | 5.00 | 5.00 |
| magnesium stearate | 0.25 | 0.25 | 0.25 | 0.25 | 0.25 | 0.25 |

Formulation 37 consists of the following ingredients (all percentages by weight):

| | |
|---|---|
| ABT-263 bis-HCl | 10.75% |
| ProSolv HD 90 ™ | 49.00% |
| mannitol | 20.00% |
| Starch 1500 ™ | 5.00% |
| sodium starch glycolate | 10.00% |
| poloxamer (Pluronic ™ F127) | 4.00% |
| colloidal silicon dioxide | 1.00% |
| sodium stearyl fumarate | 0.25% |

Tablets were prepared by one of the processes shown in Table 30.

TABLE 30
Processes used in preparing tablets

| Process | Description |
|---|---|
| I | Wet granulation; API suspended in binder |
| | solution (PVP + poloxamer) |
| II | Wet granulation; API blended intragranularly |
| III | Dry blend; directly compressed tablets |

Table 31 summarizes PK data for ABT-263 tablet formulations in dogs. F % is a measure of bioavailability.

TABLE 31
PK data for tablet formulations

| | | | | AUC | |
|---|---|---|---|---|---|
| Formulation | Process | Tₘₐₓ (h) | Cₘₐₓ (μg/ml) | (μg · h/ml) | F % |
| 26 | I | 5.3 ± 1.2 | 2.2 ± 1.0 | 24.1 | 9.6 |
| | I | 2.3 ± 0.6 | 3.5 ± 0.3 | 28.5 | 12.0 |
| | API | | | | |
| | jet-milled | | | | |
| 27 | I | 7.0 ± 6.9 | 1.8 ± 0.5 | 20.1 | 8.3 |
| | II | 3.0 ± 0.0 | 4.0 ± 1.1 | 37.7 | 16.8 |
| 28 | I | 7.3 ± 6.7 | 3.6 ± 1.6 | 47.7 | 21.5 |
| 29 | II | 6.7 ± 5.0 | 3.9 ± 2.2 | 37.5 | 14.9 |
| 30 | II | 1.8 ± 0.3 | 7.5 ± 2.3 | 60.7 | 22.6 |
| 31 | II | 2.7 ± 0.6 | 6.1 ± 2.5 | 47.6 | 20.6 |
| 32 | II | 2.3 ± 0.6 | 7.1 ± 3.2 | 42.6 | 18.6 |
| 33 | II | 4.3 ± 4.0 | 3.6 ± 1.1 | 34.5 | 13.6 |
| 34 | II | 3.7 ± 2.1 | 5.8 ± 1.5 | 48.3 | 19.2 |
| 35 | II | 3.0 ± 1.0 | 6.8 ± 1.3 | 69.9 | 25.5 |
| 36 | II | 3.0 ± 1.0 | 4.5 ± 3.2 | 51.7 | 20.4 |
| 37 | III | 3.0 ± 1.0 | 10.2 ± 2.9 | 76.2 | 31.0 |

Tablets prepared by direct compression (Process III) exhibited higher bioavailability in these dog studies than those prepared by wet granulation (Processes I and II). Tablets prepared by Process II generally provided higher bioavailability in dogs than those prepared by Process I. Adding the drug by suspending it in the binder solution also appeared to prolong the Tₘₐₓ.

Addition of a surfactant to tablets made by wet granulation did not significantly change in vivo absorption of the drug. Addition of water-soluble excipients such as mannitol appeared to enhance in vivo drug absorption.

A change in drug loading level (21.5% vs. 10.75% ABT-263 bis-HCl; 20% vs. 10% free base equivalent) did not significantly change bioavailability.

Increasing the binder (e.g., PVP) concentration for wet granulation had a tendency to reduce bioavailability.

### Example 30

### PK Studies of ABT-263 Solid Capsules in Dogs

PK studies were performed in non-fasting beagle dogs (n=3) at a single dose of 50 mg ABT-263 free base equivalent. Plasma concentrations of the drug were determined by high pressure liquid chromatography mass spectrometry (HPLC-MS) and PK parameters were calculated by standard procedures in the art.

Four capsule compositions of the invention (containing Formulations 38-41) were tested. API (ABT-263 bis-HCl in all cases) was unmilled unless otherwise indicated.

Formulation 38 consists of the following ingredients (all percentages by weight):

| | |
|---|---|
| ABT-263 bis-HCl | 10.75% |
| ProSolv HD 90 ™ | 49.00% |
| mannitol | 20.00% |
| starch 1500 | 5.00% |
| sodium starch glycolate | 10.00% |
| poloxamer (Pluronic ™ F127) | 4.00% |
| colloidal silicon dioxide | 1.00% |
| magnesium stearate | 0.25% |

Formulation 39 consists of an intragranular component and an extragranular component having the following ingredients (all percentages by weight):

Intragranular

| | |
|---|---|
| ABT-263 bis-HCl | 10.75% |
| Avicel 101 ™ | 30.00% |
| mannitol | 30.00% |
| poloxamer (Pluronic ™ F127) | 1.00% |
| hydroxypropylcellulose | 3.00% |
| sodium starch glycolate | 2.5% |

Extragranular

| | |
|---|---|
| Avicel 101 ™ | 20.00% |
| sodium starch glycolate | 2.5% |
| sodium stearyl fumarate | 0.25% |

Formulation 40 consists of the following ingredients (all percentages by weight):

| | |
|---|---|
| ABT-263 bis-HCl | 10.75% |
| ProSolv HD 90 ™ | 50.00% |
| mannitol | 30.00% |
| hydroxypropylcellulose | 3.00% |
| poloxamer (Pluronic ™ F127) | 1.00% |
| sodium starch glycolate | 5.00% |
| sodium stearyl fumarate | 0.25% |

Formulation 41 consists of the following ingredients (all percentages by weight):

| | |
|---|---|
| ABT-263 bis-HCl | 16.12% |
| Avicel 102 ™ | 50.00% |
| mannitol | 28.13% |
| sodium starch glycolate | 5.00% |
| colloidal silicon dioxide | 0.50% |
| sodium stearyl fumarate | 0.25% |

Capsule fills were prepared by one of the processes shown in Table 32.

TABLE 32
Processes used in preparing capsules

| Process | Description |
|---|---|
| II | Wet granulation; API blended intragranularly |
| IV | Dry blend encapsulation |

Table 33 summarizes PK data for ABT-263 tablet formulations in dogs. Formulation 41 was tested three times.

TABLE 33
PK data for capsule formulations

| | | | Cₘₐₓ | AUC | |
|---|---|---|---|---|---|
| Formulation | Process | Tₘₐₓ (h) | (μg/ml) | (μg · h/ml) | F % |
| 38 | IV | 4.2 ± 2.4 | 6.3 ± 1.5 | 54.1 | 21.7 |
| 39 | II | 6.7 ± 5.4 | 4.7 ± 2.4 | 51.0 | 20.3 |
| | II | 3.8 ± 1.3 | 45 ± 1.9 | 40.5 | 13.2 |
| | API jet-milled | | | | |
| 40 | III | 3.2 ± 0.8 | 6.2 ± 2.0 | 53.0 | 21.0 |
| | III | 4.7 ± 3.7 | 7.4 ± 2.0 | 74.5 | 34.2 |
| | API jet-milled | | | | |
| 41 | IV | 2.8 ± 0.7 | 2.5 ± 0.5 | 43.2 | 15.8 |
| | | 7.0 ± 4.8 | 5.0 ± 1.2 | 62.3 | 23.5 |
| | | 4.2 ± 1.5 | 6.4 ± 2.9 | 52.6 | 17.6 |

Micronization of the API by jet-milling led to improved bioavailability for capsules made by dry blending (Process IV) but not by wet granulation (Process II). Addition of poloxamer surfactant did not significantly affect bioavailability of a dry blend encapsulation formulation.

What is claimed is:

1. An orally deliverable pharmaceutical composition comprising as a sole or first active ingredient a compound of Formula I 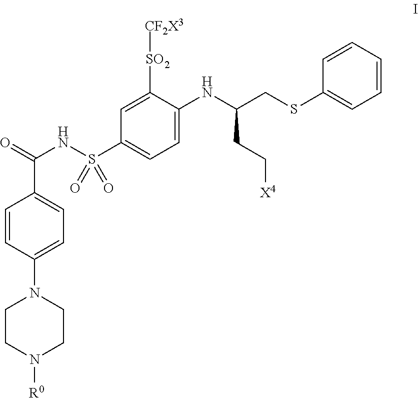
where
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is 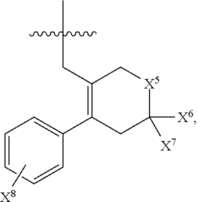
where
X⁵ is —CH₂—, —C(CH₃)₂— or —CH₂CH₂—;
X⁶ and X⁷ are both —H or both methyl; and
X⁸ is fluoro, chloro, bromo or iodo;
or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is 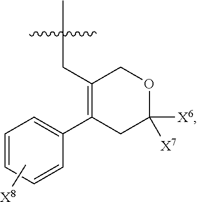
where X⁶, X⁷ and X⁸ are as above; or
(3) X⁴ is morpholin-4-yl or —N(CH₃)₂; and R⁰ is 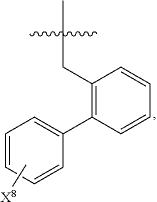
where X⁸ is as above;
or a pharmaceutically acceptable salt thereof, dispersed, in a free base equivalent amount of at least about 2.5% by weight of the composition, in a pharmaceutically acceptable carrier; wherein said active ingredient is in solid-state form and/or the composition further comprises, dispersed in the carrier, a pharmaceutically acceptable heavier-chalcogen antioxidant (HCA) in an amount effective to inhibit oxidation of the active ingredient at a thioether linkage thereof.

2. The composition of claim 1, wherein said active ingredient is present in a free base equivalent amount of at least about 5% by weight of the composition.

3. The composition of claim 1, wherein the active ingredient comprises N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl) sulfonyl)benzenesulfonamide (ABT-263) or a pharmaceutically acceptable salt thereof.

4. The composition of claim 3, wherein the active ingredient comprises ABT-263 free base or ABT-263 bis-hydrochloride salt (ABT-263 bis-HCl).

5. The composition of claim 3, wherein the carrier comprises excipients selected to provide sufficient bioavailability of ABT-263 to be therapeutically effective for promotion of apoptosis when orally administered to a non-fasting human subject in need thereof in a daily dosage amount of about 200 to about 400 mg ABT-263 free base equivalent.

6. The composition of claim 5, wherein said sufficient bioavailability is evidenced by a bioavailability of at least about 15% in a non-fasting dog model.

7. The composition of claim 5, wherein said sufficient bioavailability is evidenced by one or both of
(a) an ABT-263 AUC₀₋₂₄ of at least about 20 μg·h/ml, and/or
(b) an ABT-263 Cₘₐₓ of at least about 2.5 μg/ml,
in a single-dose non-fasting human pharmacokinetic study at an ABT-263 free base equivalent dose of about 200 to about 400 mg.

8. The composition of claim 5, wherein said sufficient bioavailability is evidenced by a steady-state ABT-263 Cₘₘ of about 1 to about 5 μg/ml and a steady-state ABT-263 Cₘₐₓ of about 3 to about 8 μg/ml in a non-fasting human pharmacokinetic study at a daily ABT-263 free base equivalent dose of about 200 to about 400 mg.

9. The composition of claim 5, wherein said sufficient bioavailability is evidenced by at least substantial bioequivalence in a human pharmacokinetic study to a prototype formulation that consists of a 25 mg/ml solution of ABT-263 bis-HCl in a mixture of 90% phosphatidylcholine+medium chain triglycerides 53/29 and 10% ethanol.

10. The composition of claim 1, wherein the carrier is liquid, having said active ingredient and a pharmaceutically acceptable HCA in an antioxidant-effective amount in solution or suspension therein.

11. The composition of claim 1, wherein the carrier is solid, having said active ingredient dispersed therein in solid-state form.

12. The composition of claim 1, wherein said active ingredient is in amorphous or crystalline form having a D₉₀ particle size not greater than about 30 μm.

13. A method for treating a disease characterized by apoptotic dysfunction and/or overexpression of an anti-apoptotic Bcl-2 family protein, comprising orally administering to a subject having the disease the composition of claim 1 in a therapeutically effective daily dosage amount.

14. The method of claim 13, wherein the disease is a neoplastic disease.

15. The method of claim 14, wherein the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof.

16. The method of claim 14, wherein the neoplastic disease is a lymphoid malignancy.

17. The method of claim 16, wherein the lymphoid malignancy is non-Hodgkin's lymphoma.

18. The method of claim 14, wherein the neoplastic disease is chronic lymphocytic leukemia or acute lymphocytic leukemia.

19. The method of claim 13, wherein said composition comprises as the sole or first active ingredient ABT-263 or a pharmaceutically acceptable salt thereof, and is administered in a daily dosage amount of about 50 to about 500 mg ABT-263 free base equivalent.

20. The method of claim 19, wherein said daily dosage amount is about 200 to about 400 mg ABT-263 free base equivalent.
